SEQUENCE LISTING (205 sequences)

SEQ ID NO: 1 - protein, 5 aa, synthetic construct
TYTMH

SEQ ID NO: 2 - protein, 17 aa, synthetic construct
FINPSSGYTKYNQNFKD

SEQ ID NO: 3 - protein, 9 aa, synthetic construct
PYGNIYFDV

SEQ ID NO: 4 - protein, 11 aa, synthetic construct
KASQNVGTAVA

SEQ ID NO: 5 - protein, 7 aa, synthetic construct
SASNRYT

SEQ ID NO: 6 - protein, 9 aa, synthetic construct
QQYSSYPLT

SEQ ID NO: 7 - protein, 118 aa, synthetic construct
QVQLQQSGAELARPGASVKMSCKASGYTFTTYTMHWIKQRPGQDLEWIGFINPSSGYTKYNQNFKDKATLTADKSSTTAYMQLNSLTSEDSAVYYCASPYGNIYFDVWGTGTTVTVSS

SEQ ID NO: 8 - protein, 107 aa, synthetic construct
DIVMTQSQKFMSTAVGDRVSITCKASQNVGTAVAWYQQKPGQSPKLLIYSASNRYTGVPDRFTGSGSGTDFTLTISNMQSEDLADYFCQQYSSYPLTFGAGTKLELK

SEQ ID NO: 9 - protein, 5 aa, synthetic construct
DYGMH

SEQ ID NO: 10 - protein, 17 aa, synthetic construct
YISSGSSTIYYADTVKG

SEQ ID NO: 11 - protein, 7 aa, synthetic construct
RPWYFDV

SEQ ID NO: 12 - protein, 16 aa, synthetic construct
RSSQSIVHSNGNTYLQ

SEQ ID NO: 13 - protein, 7 aa, synthetic construct
KVSNRFS

SEQ ID NO: 14 - protein, 9 aa, synthetic construct
FQGSHVPFT

SEQ ID NO: 15 - protein, 116 aa, synthetic construct
EVQLVESGGGLVKPGGSLKLSCAASGFTFSDYGMHWVRQAPEKGLEWVAYISSGSSTIYYADTVKGRFTISRDNAKNTLFLQMTSLRSEDTAIYYCVRRPWYFDVWGTGTTVTVSS

SEQ ID NO: 16 - protein, 112 aa, synthetic construct
DVLMTQTPLSLPVSLGDQASISCRSSQSIVHSNGNTYLQWYLQKPGQSPKLLIYKVSNRFSGVPDRFSGSGSGTDFTLKISRLEAEDLGVYYCFQGSHVPFTFGSGTKLEIK

SEQ ID NO: 17 - protein, 5 aa, synthetic construct
SYGMS

SEQ ID NO: 18 - protein, 17 aa, synthetic construct
TISSGGSYTYYPDSVKG

SEQ ID NO: 19 - protein, 5 aa, synthetic construct
HATDY

SEQ ID NO: 20 - protein, 10 aa, synthetic construct
SASSSVSYMH

SEQ ID NO: 21 - protein, 7 aa, synthetic construct
DTSKLAS

SEQ ID NO: 22 - protein, 9 aa, synthetic construct
QQWSSNPLT

SEQ ID NO: 23 - protein, 114 aa, synthetic construct
EVQLVESGGDLVKPGGSLKLSCAASGFTFSSYGMSWVRQTPDKRLEWVATISSGGSYTYYPDSVKGRFTISRDNAKNTLYLQMSSLKSEDTAMYYCARHATDYWGQGTTLTVSS

SEQ ID NO: 24 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 25 - protein, 5 aa, synthetic construct
DYGMH

SEQ ID NO: 26 - protein, 17 aa, synthetic construct
FISSVSFTIYYADTVKG

SEQ ID NO: 27 - protein, 9 aa, synthetic construct
LGPHWYFDV

SEQ ID NO: 28 - protein, 16 aa, synthetic construct
RSSQSLVYSNGNTYLH

SEQ ID NO: 29 - protein, 7 aa, synthetic construct
KVSNRFS

SEQ ID NO: 30 - protein, 8 aa, synthetic construct
SQSTPVPT

SEQ ID NO: 31 - protein, 118 aa, synthetic construct
EVRLVESGGALVRPGGSLKLSCAASGFTFSDYGMHWVRQAPEKGLEWIAFISSVSFTIYYADTVKGRFTISRDNAKNTLFLQMTSLRSEDTALYYCTRLGPHWYFDVWGTGTTVTVSS

SEQ ID NO: 32 - protein, 111 aa, synthetic construct
DVVLTQTPLSLPVSLGDQASISCRSSQSLVYSNGNTYLHWFLQKPGQSPQLLIYKVSNRFSGVPDRFSGSGSGTDFTLKISRVEAEDLGVYFCSQSTPVPTFGGGTKLEIK

SEQ ID NO: 33 - protein, 5 aa, synthetic construct
SHGMS

SEQ ID NO: 34 - protein, 17 aa, synthetic construct
TISSGGRNTYYPDSVKG

SEQ ID NO: 35 - protein, 8 aa, synthetic construct
HSGDAMDY

SEQ ID NO: 36 - protein, 11 aa, synthetic construct
RASQSIGTNIH

SEQ ID NO: 37 - protein, 7 aa, synthetic construct
YASESIS

SEQ ID NO: 38 - protein, 8 aa, synthetic construct
QQSKSWPT

SEQ ID NO: 39 - protein, 117 aa, synthetic construct
EVQLVESGGDLVKPGGYLRLSCAASGFTFSSHGMSWVRQTPDKRLEWVATISSGGRNTYYPDSVKGRFTISRDNAKNILYLQMSSLKSEDTAMFYCTRHSGDAMDYWGQGTSVTVSS

SEQ ID NO: 40 - protein, 106 aa, synthetic construct
DILLTQSPAILSVSPGERVSFSCRASQSIGTNIHWYQQRTNGSPRLLIKYASESISGIPSRFSGSGSGTDFTLSINSVESEDIADYYCQQSKSWPTFGGGTKLEIK

SEQ ID NO: 41 - protein, 5 aa, synthetic construct
TYGLS

SEQ ID NO: 42 - protein, 17 aa, synthetic construct
WINTFSGVPIYADDFKG

SEQ ID NO: 43 - protein, 9 aa, synthetic construct
WGIRQRGDY

SEQ ID NO: 44 - protein, 10 aa, synthetic construct
SASSSVSYLH

SEQ ID NO: 45 - protein, 7 aa, synthetic construct
DTSKLAS

SEQ ID NO: 46 - protein, 9 aa, synthetic construct
QQWTSNPPT

SEQ ID NO: 47 - protein, 118 aa, synthetic construct
QIQLVQSGPELKMPGETVKISCKASGYTFTTYGLSWVKQAPGKGLKWMGWINTFSGVPIYADDFKGRFAFSLETSASTAYLQINNLKNEDTATYFCARWGIRQRGDYWGQGATLTVSS

SEQ ID NO: 48 - protein, 106 aa, synthetic construct
QIVLTQSPAIMSASPGEKVTMTCSASSSVSYLHWYQQKSGTSPKRWIYDTSKLASGVPVRFSGSGSETSYSLTISSMEAEDAATYYCQQWTSNPPTFGGGTKLEIK

SEQ ID NO: 49 - protein, 5 aa, synthetic construct
SYAMS

SEQ ID NO: 50 - protein, 17 aa, synthetic construct
TISDGGSYTYYPDNVKG

SEQ ID NO: 51 - protein, 6 aa, synthetic construct
RGYFDY

SEQ ID NO: 52 - protein, 11 aa, synthetic construct
KASQDINSYLS

SEQ ID NO: 53 - protein, 7 aa, synthetic construct
RANRLVD

SEQ ID NO: 54 - protein, 8 aa, synthetic construct
LQYDELYT

SEQ ID NO: 55 - protein, 115 aa, synthetic construct
EVQLVESGGGLVKPGGSLKLSCAASGFTFSSYAMSWVRQTPEKRLEWVATISDGGSYTYYPDNVKGRFTISRDNAKNNLYLQMSHLKSEDTAMYYCARRGYFDYWGQGTTLTVSS

SEQ ID NO: 56 - protein, 106 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDELYTFGGGTKLEIK

SEQ ID NO: 57 - protein, 5 aa, synthetic construct
SYAMS

SEQ ID NO: 58 - protein, 17 aa, synthetic construct
TISDGGSYTYYPDNVKG

SEQ ID NO: 59 - protein, 6 aa, synthetic construct
RGYFDY

SEQ ID NO: 60 - protein, 11 aa, synthetic construct
KASQDINKYIA

SEQ ID NO: 61 - protein, 7 aa, synthetic construct
YTSTLQP

SEQ ID NO: 62 - protein, 8 aa, synthetic construct
LQYDNLYT

SEQ ID NO: 63 - protein, 115 aa, synthetic construct
EVQLVESGGGLVKPGGSLKLSCAASGFTFSSYAMSWVRQTPEKRLEWVATISDGGSYTYYPDNVKGRFTISRDNAKNNLYLQMSHLKSEDTAMYYCTRRGYFDYWGQGTTLTVSS

SEQ ID NO: 64 - protein, 106 aa, synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLEIK

SEQ ID NO: 65 - protein, 5 aa, synthetic construct
YYVTH

SEQ ID NO: 66 - protein, 17 aa, synthetic construct
YINPYNDGTMFNEKFKG

SEQ ID NO: 67 - protein, 9 aa, synthetic construct
GGVRRYFDV

SEQ ID NO: 68 - protein, 11 aa, synthetic construct
RASQDIGSNLN

SEQ ID NO: 69 - protein, 7 aa, synthetic construct
ATSSLDS

SEQ ID NO: 70 - protein, 9 aa, synthetic construct
LQYASFPYT

SEQ ID NO: 71 - protein, 118 aa, synthetic construct
EVQLQQSGPELVKPGASVKMSCKASGYTFTYYVTHWVRQKPGQGLEWIGYINPYNDGTMFNEKFKGKATLTSDKSSSTAYMEFSSLTSEDSAVFYCARGGVRRYFDVWGAGTTVTVSS

SEQ ID NO: 72 - protein, 107 aa, synthetic construct
DIQMTQSPSSLSASLGERVSLTCRASQDIGSNLNWLQQKPDGTIKRLIYATSSLDSGVPKRFSGSRSGSDYSLTINSLESEDFVDYYCLQYASFPYTFGGGTKLEIK

SEQ ID NO: 73 - protein, 5 aa, synthetic construct
YYILH

SEQ ID NO: 74 - protein, 17 aa, synthetic construct
YFNPYNDGNKFNERFRG

SEQ ID NO: 75 - protein, 9 aa, synthetic construct
GGVRRYFDV

SEQ ID NO: 76 - protein, 11 aa, synthetic construct
RASQDIGSNLN

SEQ ID NO: 77 - protein, 7 aa, synthetic construct
ATFSLDS

SEQ ID NO: 78 - protein, 9 aa, synthetic construct
LQYASFPYT

SEQ ID NO: 79 - protein, 118 aa, synthetic construct
EVQLQQSGPELVKPGASVKMSCKASGYTFTYYILHWVKQKPGQGLEWIGYFNPYNDGNKFNERFRGKATLTSDKSSSTAYMELNSLTSEDSAVYFCARGGVRRYFDVWGAGTTVTVSS

SEQ ID NO: 80 - protein, 107 aa, synthetic construct
DIQMTQSPSSLSASLGERVSLTCRASQDIGSNLNWLQQEPDGTIKRLISATFSLDSGVPKRFSGSRSGSDYSLTISSLESEDFVNYYCLQYASFPYTFGGGTKLEIK

SEQ ID NO: 81 - protein, 5 aa, synthetic construct
NYAMH

SEQ ID NO: 82 - protein, 17 aa, synthetic construct
GISWNSGSIGYADSVKG

SEQ ID NO: 83 - protein, 14 aa, synthetic construct
DSRGYGDYSRGGAY

SEQ ID NO: 84 - protein, 11 aa, synthetic construct
RASQSVSSNLA

SEQ ID NO: 85 - protein, 7 aa, synthetic construct
GASTRAT

SEQ ID NO: 86 - protein, 9 aa, synthetic construct
QQYNNWPWT

SEQ ID NO: 87 - protein, 123 aa, synthetic construct
EVQLVESGGGLVQPGRSLRLSCAASGFTFDNYAMHWVRQAPGKGLEWVSGISWNSGSIGYADSVKGRFTISRDNAKNSLYLQMNSLRAEDTALYYCAKDSRGYGDYSRGGAYWGQGTLVTVSS

SEQ ID NO: 88 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 89 - protein, 5 aa, synthetic construct
TYAMN

SEQ ID NO: 90 - protein, 19 aa, synthetic construct
RIRSKYNNYATYYAASVKG

SEQ ID NO: 91 - protein, 14 aa, synthetic construct
HGNFGNSYVSWFAY

SEQ ID NO: 92 - protein, 14 aa, synthetic construct
RSSTGAVTTSNYAN

SEQ ID NO: 93 - protein, 7 aa, synthetic construct
GTNKRAP

SEQ ID NO: 94 - protein, 9 aa, synthetic construct
ALWYSNLWV

SEQ ID NO: 95 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 96 - protein, 109 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 97 - protein, 5 aa, synthetic construct
SYIMH

SEQ ID NO: 98 - protein, 17 aa, synthetic construct
YIYPGNGGTNYNQKFKD

SEQ ID NO: 99 - protein, 9 aa, synthetic construct
WGLRQGFAY

SEQ ID NO: 100 - protein, 11 aa, synthetic construct
KASQNVGGNVA

SEQ ID NO: 101 - protein, 7 aa, synthetic construct
STSYRNS

SEQ ID NO: 102 - protein, 9 aa, synthetic construct
QQYNSYPFT

SEQ ID NO: 103 - protein, 118 aa, synthetic construct
QAYLQQSGAELVRSGASVKMSCKASGYTFTSYIMHWVKQTPGQGLEWIGYIYPGNGGTNYNQKFKDKATLSADTSSSTAYMQINSLTSEDSAVYFCARWGLRQGFAYWGQGTLVSVSA

SEQ ID NO: 104 - protein, 107 aa, synthetic construct
DIVMTQSQKFMSTSVGDRVSVTCKASQNVGGNVAWYQQKPGQSPKALIYSTSYRNSGVPDRFTGSGSGTDFTLTISNVQSEDLAEYFCQQYNSYPFTFGSGTKLEIR

SEQ ID NO: 105 - protein, 5 aa, synthetic construct
SSTIH

SEQ ID NO: 106 - protein, 17 aa, synthetic construct
YIVPSTGYTNYNQKFKD

SEQ ID NO: 107 - protein, 12 aa, synthetic construct
SDGYPFGYSMAY

SEQ ID NO: 108 - protein, 11 aa, synthetic construct
HASQNINVWLS

SEQ ID NO: 109 - protein, 7 aa, synthetic construct
KASNLHT

SEQ ID NO: 110 - protein, 9 aa, synthetic construct
QQGQSYPFT

SEQ ID NO: 111 - protein, 121 aa, synthetic construct
QVQLQQSGAELARPGASVKMSCKASGYTFTSSTIHWVKQRPGQGLAWIGYIVPSTGYTNYNQKFKDKATLTADKSSSTAYMQLTSLTSEDSAVYYCARSDGYPFGYSMAYWGQGTSVTVSS

SEQ ID NO: 112 - protein, 107 aa, synthetic construct
DIQMNQSPSSLSASLGDTITITCHASQNINVWLSWYHQKPGNIPKLLIYKASNLHTGVPSRFSGSGSGTGFTLTISRLQPEDIATYYCQQGQSYPFTFGSGTKLEIK

SEQ ID NO: 113 - protein, 5 aa, synthetic construct
SYSVY

SEQ ID NO: 114 - protein, 16 aa, synthetic construct
VIWAGGSTNYNSALMS

SEQ ID NO: 115 - protein, 9 aa, synthetic construct
RWLPRGLDF

SEQ ID NO: 116 - protein, 23 aa, synthetic construct
DIVMTQSHKFMSTSVGDRVSITC

SEQ ID NO: 117 - protein, 7 aa, synthetic construct
WASTRHT

SEQ ID NO: 118 - protein, 8 aa, synthetic construct
QQYSTYYT

SEQ ID NO: 119 - protein, 117 aa, synthetic construct
QVQLKESGPGLVAPSQSLSITCTVSGFSLSSYSVYWVRQPPGKGLEWLGVIWAGGSTNYNSALMSRLSINKDKSKSQVFLKMNSLQTDDSAIYYCAMRWLPRGLDFWGQGTSVTVSS

SEQ ID NO: 120 - protein, 106 aa, synthetic construct
DIVMTQSHKFMSTSVGDRVSITCKASQDVGTAVAWYQQKPGQSPKLLIYWASTRHTGVPDRFTGSGSGTDFTLTISNVQSEDLADYFCQQYSTYYTFGGGTKLEIK

SEQ ID NO: 121 - protein, 5 aa, synthetic construct
NYVIH

SEQ ID NO: 122 - protein, 17 aa, synthetic construct
YFNPYNDGSKYNEKFKG

SEQ ID NO: 123 - protein, 9 aa, synthetic construct
GGVRRYFDV

SEQ ID NO: 124 - protein, 11 aa, synthetic construct
RASQDIGDNLN

SEQ ID NO: 125 - protein, 7 aa, synthetic construct
ATFSLDS

SEQ ID NO: 126 - protein, 9 aa, synthetic construct
LHYASFPYT

SEQ ID NO: 127 - protein, 118 aa, synthetic construct
EVQLQQSGPELVKPGASVKMSCKASGYTFSNYVIHWVKQKPGQGLEWIGYFNPYNDGSKYNEKFKGKATLTSDKSSSTAYMELSSLTSEDSAVYYCARGGVRRYFDVWGAGTTVTVSS

SEQ ID NO: 128 - protein, 107 aa, synthetic construct
DIQMTQSPSSLSASLGERVSLTCRASQDIGDNLNWLQQEPDGTIKRLIYATFSLDSGVPKRFSGSRSGSVYSLTISRLESEDFVDYFCLHYASFPYTFGGGTKLEIK

SEQ ID NO: 129 - protein, 5 aa, synthetic construct
NYAVT

SEQ ID NO: 130 - protein, 16 aa, synthetic construct
VIWGDGSTYYHSALVS

SEQ ID NO: 131 - protein, 8 aa, synthetic construct
GGYSPFAY

SEQ ID NO: 132 - protein, 16 aa, synthetic construct
RSSQSLVHSNGNTYLH

SEQ ID NO: 133 - protein, 7 aa, synthetic construct
KVSNRFS

SEQ ID NO: 134 - protein, 9 aa, synthetic construct
SQNTHVPFT

SEQ ID NO: 135 - protein, 116 aa, synthetic construct
QVQLKESGPGLVAPSQSLSITCTVSGFSLTNYAVTWVRQPPGKGLEWLGVIWGDGSTYYHSALVSRLSITKDNSRTQVLLKLNSLQTDDTARYYCAKGGYSPFAYWGQGTLVTVSA

SEQ ID NO: 136 - protein, 112 aa, synthetic construct
DVVMTQTPLSLPVSLGDQASISCRSSQSLVHSNGNTYLHWYLQKPGQSPKLLIFKVSNRFSGVPDRFSGSGSGTDFTLKISRVEAEDLGVYFCSQNTHVPFTFGSGTKLEIK

SEQ ID NO: 137 - protein, 5 aa, synthetic construct
DTYMH

SEQ ID NO: 138 - protein, 17 aa, synthetic construct
RIDPANGNTKYDPKFQG

SEQ ID NO: 139 - protein, 12 aa, synthetic construct
DYGNYEDYAMDY

SEQ ID NO: 140 - protein, 15 aa, synthetic construct
KASQSVDYDGDSYMN

SEQ ID NO: 141 - protein, 7 aa, synthetic construct
AASNLES

SEQ ID NO: 142 - protein, 9 aa, synthetic construct
QQSNEDPPT

SEQ ID NO: 143 - protein, 121 aa, synthetic construct
EVQLQQSGAELVKPGASVKLSCTASGFNIKDTYMHWVKQRPEQGLEWIGRIDPANGNTKYDPKFQGKATITADTSSNTAYLQLSSLTSEDTAVYYCARDYGNYEDYAMDYWGQGTSVTVSS

SEQ ID NO: 144 - protein, 111 aa, synthetic construct
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPPTFGGGTKLEIK

SEQ ID NO: 145 - protein, 5 aa, synthetic construct
YYIMH

SEQ ID NO: 146 - protein, 17 aa, synthetic construct
YINPYNDGAYYSARFKG

SEQ ID NO: 147 - protein, 9 aa, synthetic construct
GGVRRYFDV

SEQ ID NO: 148 - protein, 11 aa, synthetic construct
RASQDIGSNLI

SEQ ID NO: 149 - protein, 7 aa, synthetic construct
ATFNLDS

SEQ ID NO: 150 - protein, 9 aa, synthetic construct
LQYASSPYT

SEQ ID NO: 151 - protein, 118 aa, synthetic construct
EVQLQQSGPELVKPGASVKMSCKASGYTFTYYIMHWVKQEPGQGLQWIGYINPYNDGAYYSARFKGKATLTSDKSSSTAYMDLTSLTSEDSAVYYCARGGVRRYFDVWGAGTTVTVSS

SEQ ID NO: 152 - protein, 107 aa, synthetic construct
DIQMTQSPSSLSASLGERVILTCRASQDIGSNLIWLQQKPDGTIKRLIYATFNLDSGVSKRFSGSRSGSDYSLTISSLDSEDFVDYYCLQYASSPYTFGGGTKLEIK

SEQ ID NO: 153 - protein, 5 aa, synthetic construct
TYVMH

SEQ ID NO: 154 - protein, 17 aa, synthetic construct
YINPYNDGTKYNEKFKG

SEQ ID NO: 155 - protein, 9 aa, synthetic construct
GGVRRYFDV

SEQ ID NO: 156 - protein, 11 aa, synthetic construct
RASQDIGSNLN

SEQ ID NO: 157 - protein, 7 aa, synthetic construct
ATFTLDS

SEQ ID NO: 158 - protein, 9 aa, synthetic construct
LQYASFPYT

SEQ ID NO: 159 - protein, 118 aa, synthetic construct
EVQLQHSGPELVKPGASVKMSCKASGYTFTTYVMHWVKQKPGQGLEWIGYINPYNDGTKYNEKFKGKATLTSDKSSSTAYMELSSLTSEDSAVYYCARGGVRRYFDVWGAGTTVTVSS

SEQ ID NO: 160 - protein, 107 aa, synthetic construct
DIQMTQSPSSLSASLGERVSLTCRASQDIGSNLNWLQQEPNGTIKRLIYATFTLDSGVPKRFSGSRSGSDYSLTISSLESEDFVDYYCLQYASFPYTFGGGTKLEIK

SEQ ID NO: 161 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 226 aa, synthetic construct
KTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 164 - protein, 5 aa, synthetic construct
TGGGG

SEQ ID NO: 165 - protein, 5 aa, synthetic construct
GGGGS

SEQ ID NO: 166 - protein, 5 aa, synthetic construct
SGGGG

SEQ ID NO: 167 - protein, 15 aa, synthetic construct
GGGGSGGGGSGGGGS

SEQ ID NO: 168 - protein, 4 aa, synthetic construct
GGGS

SEQ ID NO: 169 - protein, 5 aa, synthetic construct
FYIMH

SEQ ID NO: 170 - protein, 17 aa, synthetic construct
YINPYNDGTKYNAKFKG

SEQ ID NO: 171 - protein, 9 aa, synthetic construct
GGVRRYFDV

SEQ ID NO: 172 - protein, 11 aa, synthetic construct
RSSQDIGSNLN

SEQ ID NO: 173 - protein, 7 aa, synthetic construct
ATFSLDS

SEQ ID NO: 174 - protein, 9 aa, synthetic construct
LQYGSFPYT

SEQ ID NO: 175 - protein, 118 aa, synthetic construct
EVQLQQSGPELVKPGASVKMSCKASGYTFTFYIMHWVKQKPGQGPEWIGYINPYNDGTKYNAKFKGKATLSSDRSSSTAYMELSSLTSEDSAVYYCARGGVRRYFDVWGAGTPVTVSS

SEQ ID NO: 176 - protein, 107 aa, synthetic construct
DIQMTQSPSSLSASLGERVSLTCRSSQDIGSNLNWLQQEPDGTIKRLIYATFSLDSGVPKRFSGSRSGSHYSLTISSLESEDFVVYYCLQYGSFPYTFGGGTKLEIK

SEQ ID NO: 177 - protein, 5 aa, synthetic construct
SYVIH

SEQ ID NO: 178 - protein, 17 aa, synthetic construct
YFNPYNDGTKYNEEFKG

SEQ ID NO: 179 - protein, 9 aa, synthetic construct
GGVRRYFDV

SEQ ID NO: 180 - protein, 11 aa, synthetic construct
RASQDIGSNLN

SEQ ID NO: 181 - protein, 7 aa, synthetic construct
ATFGLDS

SEQ ID NO: 182 - protein, 9 aa, synthetic construct
QQYASFPYT

SEQ ID NO: 183 - protein, 118 aa, synthetic construct
EVQLQQSGPELVKPGASVKMSCKASGYTFTSYVIHWVKQKTGQGLEWIGYFNPYNDGTKYNEEFKGKATLTSDKSSNTAYMELNSLTSDDSAVYFCARGGVRRYFDVWGAGTTVTVSS

SEQ ID NO: 184 - protein, 107 aa, synthetic construct
DIQMTQSPSSLSASLGERVSLTCRASQDIGSNLNWLQQEPDGTIKRLIYATFGLDSGVPKRFSGSRSGSDYSLTISSLESEDFVDYYCQQYASFPYTFGGGTKLEIK

SEQ ID NO: 185 - protein, 5 aa, synthetic construct
GYTMN

SEQ ID NO: 186 - protein, 17 aa, synthetic construct
LINPYNGGTRYNQKFKD

SEQ ID NO: 187 - protein, 9 aa, synthetic construct
WLLRYAMDY

SEQ ID NO: 188 - protein, 11 aa, synthetic construct
KASQNVGNAVA

SEQ ID NO: 189 - protein, 7 aa, synthetic construct
SASNRKN

SEQ ID NO: 190 - protein, 9 aa, synthetic construct
QQYSDSSWT

SEQ ID NO: 191 - protein, 118 aa, synthetic construct
EVQLQQSGPELVKPGASMKISCKASGYSFTGYTMNWVKQSHGKNLEWIGLINPYNGGTRYNQKFKDKATLTVDKSSSTAYMELLSLTSEDSAVYYCARWLLRYAMDYWGQGTSVTVSS

SEQ ID NO: 192 - protein, 107 aa, synthetic construct
DIVMTQSQKFMSTSVGDRVSITCKASQNVGNAVAWYQQKPGHSPKLLISSASNRKNGVPDRFTGSGSGTDFTLTVSNMQSEDLADYFCQQYSDSSWTFGGGTKLEIK

SEQ ID NO: 193 - protein, 5 aa, synthetic construct
GYTMN

SEQ ID NO: 194 - protein, 17 aa, synthetic construct
LINPYNSDTNYAQKLQG

SEQ ID NO: 195 - protein, 9 aa, synthetic construct
VALRVALDY

SEQ ID NO: 196 - protein, 11 aa, synthetic construct
KASQNVATHVG

SEQ ID NO: 197 - protein, 7 aa, synthetic construct
SASYRYS

SEQ ID NO: 198 - protein, 9 aa, synthetic construct
QQYNRYPYT

SEQ ID NO: 199 - protein, 118 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 200 - protein, 107 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 201 - protein, 119 aa, synthetic construct
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 202 - protein, 109 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVL

SEQ ID NO: 203 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 204 - protein, 42 aa, synthetic construct
KRGRKKLLYIFKQPFMRPVQTTQEEDGCSCRFPEEEEGGCEL

SEQ ID NO: 205 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0368734 A1
(43) Pub. Date: Dec. 4, 2025

(54) NOVEL ANTI-GPRC5D ANTIBODIES, BISPECIFIC ANTIGEN BINDING MOLECULES THAT BIND GPRC5D AND CD3, AND USES THEREOF

(71) Applicant: ANTENGENE BIOLOGICS LIMITED, Shanghai (CN)

(72) Inventors: Hui YUWEN, Shanghai (CN); Tengteng LI, Shanghai (CN); Yijing REN, Shanghai (CN); Peng CHEN, Shanghai (CN); Bing HOU, Shanghai (CN); Bo SHAN, Shanghai (CN); Jay MEI, Shanghai (CN)

(21) Appl. No.: 18/869,284

(22) PCT Filed: May 25, 2023
(86) PCT No.: PCT/CN2023/096296

§ 371 Date: Nov. 26, 2024

(30) Foreign Application Priority Data
May 27, 2022 (WO) PCT/CN2022/095719

(51) Int. Cl. C07K 16/28; A61K 39/00; A61K 40/11; A61K 40/31; A61K 40/42; A61P 35/00; C12N 5/0783; G01N 33/574

(52) U.S. Cl. CPC C07K 16/28; A61K 40/11; A61K 40/31; A61K 40/4202; A61P 35/00; C07K 16/2809; C12N 5/0636; G01N 33/57484; A61K 2039/505; C07K 2317/24; C07K 2317/31; C07K 2317/55; C07K 2317/622; C07K 2317/73; C07K 2317/92; C12N 2510/00

(57) ABSTRACT

The present disclosure provides anti-GPRC5D antibodies or antigen-binding fragments thereof, bispecific antigen binding molecules that bind GPRC5D and CD3, isolated polynucleotides encoding the same, pharmaceutical composition comprising the same and the uses thereof.

### FIELD OF THE INVENTION

The present disclosure generally relates to novel anti-GPRC5D antibodies and antibody fragments thereof, bispecific antigen binding molecules that bind GPRC5D and CD3, and uses of the same.

### BACKGROUND

G-protein coupled receptor, class C, group 5, member D (GPRC5D) is an orphan, atypical, class C G-protein coupled receptor (GPCR) first identified in 2001 (Brauner-Osborne et al., Biochim Biophys Acta. 1518(3):237-248, 2001). GPRC5D is a specific surface protein expressed by plasma cells in multiple myeloma, and might be a relevant target on plasma cells in autoimmunity as well. It has been reported that GPRC5D is associated with prognosis and tumor load in multiple myeloma patients (Atamaniuk, J., et al., Eur J Clin Invest, 42(9): 953-960, 2012; and Cohen, Y., et al., Hematology, 18(6): 348-351, 2013). Various anti-GPRC5D antibodies have been described in the field, for example, in WO2018017786A2 and WO2021018925A1.

The CD3 (cluster of differentiation 3) T-cell co-receptor is a protein complex and is composed of four distinct chains, a CD3gamma chain, a CD3delta chain, and two CD3epsilon chains. These chains associate with a molecule known as the T-cell receptor (TCR) and the zeta-chain to generate activation signal in T lymphocytes. The TCR, zeta-chain, and CD3 molecules together form the TCR-CD3 complex, in which TCR as a subunit recognizes and binds to antigen, and CD3 as a subunit transfers and conveys the antigen-stimulation to signaling pathway, and ultimately regulates T-cell activity. The CD3 protein is virtually present in all T cells. A more recent application of CD3 antibodies is in the form of bispecific antibodies, binding CD3 on the one hand and a tumor cell antigen on the other hand. The simultaneous binding of such an antibody to both of its targets will force a temporary interaction between target cell and T cell, causing activation of any cytotoxic T cell and subsequent lysis of the target cell.

Needs remain for additional drugs to treat cancer, particularly multiple myeloma.

### SUMMARY OF THE INVENTION

Throughout the present disclosure, the articles “a”, “an”, and “the” are used herein to refer to one or to more than one (i.e., to at least one) of the grammatical object of the article. By way of example, “an antibody” means one antibody or more than one antibody.

In one respect, the present disclosure provides an antibody or antigen-binding fragment thereof which binds to GPRC5D, comprising:
one or two or three heavy chain complementarity determining regions (HCDR1, HCDR2 and/or HCDR3) contained within any one of the heavy chain variable (VH) region sequences selected from the group consisting of SEQ ID NOs: 7, 15, 23, 31, 39, 47, 55, 63, 71, 79, 103, 111, 119, 127, 135, 143, 151, 159, 175, 183, and 191; and/or
one or two or three light chain complementarity determining regions (LCDR1, LCDR2 and LCDR3) contained within any one of the light chain variable (VL) region sequences selected from the group consisting of SEQ ID NOs: 8, 16, 24, 32, 40, 48, 56, 64, 72, 80, 104, 112, 120, 128, 136, 144, 152, 160, 176, 184, and 192.

In some embodiments, the antibody or antigen-binding fragment thereof comprises at least one heavy or light chain complementarity determining region (CDR) comprising an amino acid sequence selected from the group consisting of SEQ ID NOs: 1, 2, 3, 4, 5, 6, 9, 10, 11, 12, 13, 14, 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 97, 98, 99, 100, 101, 102, 105, 106, 107, 108, 109, 110, 113, 114, 115, 116, 117, 118, 121, 122, 123, 124, 125, 126, 129, 130, 131, 132, 133, 134, 137, 138, 139, 140, 141, 142, 145, 146, 147, 148, 149, 150, 153, 154, 155, 156, 157, 158, 169, 170, 171, 172, 173, 174, 177, 178, 179, 180, 181, 182, 185, 186, 187, 188, 189, and 190.

In some embodiments, the antibody or antigen-binding fragment thereof of the present disclosure comprises a VH region comprising one or two or three of HCDR1, HCDR2 and HCDR3 comprising an amino acid sequence selected from the group consisting of SEQ ID NOs: 1, 2, 3, 9, 10, 11, 17, 18, 19, 25, 26, 27, 33, 34, 35, 41, 42, 43, 49, 50, 51, 57, 58, 59, 65, 66, 67, 73, 74, 75, 97, 98, 99, 105, 106, 107, 113, 114, 115, 121, 122, 123, 129, 130, 131, 137, 138, 139, 145, 146, 147, 153, 154, 155, 169, 170, 171, 177, 178, 179, 185, 186, and 187.

In some embodiments, the antibody or antigen-binding fragment thereof of the present disclosure comprises a VL region comprising one or two or three of LCDR1, LCDR2 and LCDR3 comprising an amino acid sequence selected from the group consisting of SEQ ID NOs: 4, 5, 6, 12, 13, 14, 20, 21, 22, 28, 29, 30, 36, 37, 38, 44, 45, 46, 52, 53, 54, 60, 61, 62, 68, 69, 70, 76, 77, 78, 100, 101, 102, 108, 109, 110, 116, 117, 118, 124, 125, 126, 132, 133, 134, 140, 141, 142, 148, 149, 150, 156, 157, 158, 172, 173, 174, 180, 181, 182, 188, 189, and 190.

In some embodiments, the antibody or antigen-binding fragment thereof of the present disclosure comprises:
i. a HCDR1 comprising an amino acid sequence selected from the group consisting of SEQ ID NOs: 1, 9, 17, 25, 33, 41, 49, 57, 65, 73, 97, 105, 113, 121, 129, 137, 145, 153, 169, 177, and 185;
ii. a HCDR2 comprising an amino acid sequence selected from the group consisting of SEQ ID NOs: 2, 10, 18, 26, 34, 42, 50, 58, 66, 74, 98, 106, 114, 122, 130, 138, 146, 154, 170, 178, and 186; and
iii. a HCDR3 comprising an amino acid sequence selected from the group consisting of SEQ ID NOs: 3, 11, 19, 27, 35, 43, 51, 59, 67, 75, 99, 107, 115, 123, 131, 139, 147, 155, 171, 179, and 187.

In some embodiments, the antibody or antigen-binding fragment thereof of the present disclosure comprises:
i. a LCDR1 comprising an amino acid sequence selected from the group consisting of SEQ ID NOs: 4, 12, 20, 28, 36, 44, 52, 60, 68, 76, 100, 108, 116, 124, 132, 140, 148, 156, 172, 180, and 188;
ii. a LCDR2 comprising an amino acid sequence selected from the group consisting of SEQ ID NOs: 5, 13, 21, 29, 37, 45, 53, 61, 69, 77, 101, 109, 117, 125, 133, 141, 149, 157, 173, 181, and 189; and
iii. a LCDR3 comprising an amino acid sequence selected from the group consisting of SEQ ID NOs: 6, 14, 22, 30, 38, 46, 54, 62, 70, 78, 102, 110, 118, 126, 134, 142, 150, 158, 174, 182, and 190.

In some embodiments, the antibody or antigen-binding fragment thereof of the present disclosure comprises:
i. a HCDR1 comprising an amino acid sequence as set forth in SEQ ID NO: 1, a HCDR2 comprising an amino acid sequence as set forth in SEQ ID NO: 2, and a HCDR3 comprising an amino acid sequence as set forth in SEQ ID NO: 3;
ii. a HCDR1 comprising an amino acid sequence as set forth in SEQ ID NO: 9, a HCDR2 comprising an amino acid sequence as set forth in SEQ ID NO: 10, and a HCDR3 comprising an amino acid sequence as set forth in SEQ ID NO: 11;
iii. a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
iv. a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
v. a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
vi. a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
vii. a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
viii. a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
ix. a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
x. a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
xi. a HCDR1 comprising an amino acid sequence as set forth in SEQ ID NO: 97, a HCDR2 comprising an amino acid sequence as set forth in SEQ ID NO: 98, and a HCDR3 comprising an amino acid sequence as set forth in SEQ ID NO: 99;
xii. a HCDR1 comprising an amino acid sequence as set forth in SEQ ID NO: 105, a HCDR2 comprising an amino acid sequence as set forth in SEQ ID NO: 106, and a HCDR3 comprising an amino acid sequence as set forth in SEQ ID NO: 107;
xiii. a HCDR1 comprising an amino acid sequence as set forth in SEQ ID NO: 113, a HCDR2 comprising an amino acid sequence as set forth in SEQ ID NO: 114, and a HCDR3 comprising an amino acid sequence as set forth in SEQ ID NO: 115;
xiv. a HCDR1 comprising an amino acid sequence as set forth in SEQ ID NO: 121, a HCDR2 comprising an amino acid sequence as set forth in SEQ ID NO: 122, and a HCDR3 comprising an amino acid sequence as set forth in SEQ ID NO: 123;
xv. a HCDR1 comprising an amino acid sequence as set forth in SEQ ID NO: 129, a HCDR2 comprising an amino acid sequence as set forth in SEQ ID NO: 130, and a HCDR3 comprising an amino acid sequence as set forth in SEQ ID NO: 131;
xvi. a HCDR1 comprising an amino acid sequence as set forth in SEQ ID NO: 137, a HCDR2 comprising an amino acid sequence as set forth in SEQ ID NO: 138, and a HCDR3 comprising an amino acid sequence as set forth in SEQ ID NO: 139;
xvii. a HCDR1 comprising an amino acid sequence as set forth in SEQ ID NO: 145, a HCDR2 comprising an amino acid sequence as set forth in SEQ ID NO: 146, and a HCDR3 comprising an amino acid sequence as set forth in SEQ ID NO: 147;
xviii. a HCDR1 comprising an amino acid sequence as set forth in SEQ ID NO: 153, a HCDR2 comprising an amino acid sequence as set forth in SEQ ID NO: 154, and a HCDR3 comprising an amino acid sequence as set forth in SEQ ID NO: 155;
xix. a HCDR1 comprising an amino acid sequence as set forth in SEQ ID NO: 169, a HCDR2 comprising an amino acid sequence as set forth in SEQ ID NO: 170, and a HCDR3 comprising an amino acid sequence as set forth in SEQ ID NO: 171;
xx. a HCDR1 comprising an amino acid sequence as set forth in SEQ ID NO: 177, a HCDR2 comprising an amino acid sequence as set forth in SEQ ID NO: 178, and a HCDR3 comprising an amino acid sequence as set forth in SEQ ID NO: 179; or
xxi. a HCDR1 comprising an amino acid sequence as set forth in SEQ ID NO: 185, a HCDR2 comprising an amino acid sequence as set forth in SEQ ID NO: 186, and a HCDR3 comprising an amino acid sequence as set forth in SEQ ID NO: 187.

In some embodiments, the antibody or antigen-binding fragment thereof of the present disclosure comprises:
i. a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
ii. a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
iii. a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
iv. a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
v. a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
vi. a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vii. a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
viii. a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
ix. a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
x. a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
xi. a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142;
xvii. a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150;
xviii. a LCDR1 comprising an amino acid sequence as set forth in SEQ ID NO: 156, a LCDR2 comprising an amino acid sequence as set forth in SEQ ID NO: 157, and a LCDR3 comprising an amino acid sequence as set forth in SEQ ID NO: 158;
xix. a LCDR1 comprising an amino acid sequence as set forth in SEQ ID NO: 172, a LCDR2 comprising an amino acid sequence as set forth in SEQ ID NO: 173, and a LCDR3 comprising an amino acid sequence as set forth in SEQ ID NO: 174;
xx. a LCDR1 comprising an amino acid sequence as set forth in SEQ ID NO: 180, a LCDR2 comprising an amino acid sequence as set forth in SEQ ID NO: 181, and a LCDR3 comprising an amino acid sequence as set forth in SEQ ID NO: 182; or
xxi. a LCDR1 comprising an amino acid sequence as set forth in SEQ ID NO: 188, a LCDR2 comprising an amino acid sequence as set forth in SEQ ID NO: 189, and a LCDR3 comprising an amino acid sequence as set forth in SEQ ID NO: 190.

In some embodiments, the antibody or antigen-binding fragment thereof of the present disclosure comprises:
i. a HCDR1 comprising an amino acid sequence as set forth in SEQ ID NO: 1, a HCDR2 comprising an amino acid sequence as set forth in SEQ ID NO: 2, a HCDR3 comprising an amino acid sequence as set forth in SEQ ID NO: 3, a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
ii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
iii. a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
iv. a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
v. a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
vi. a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vii. a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
viii. a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
ix. a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
x. a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
xi. a HCDR1 comprising an amino acid sequence as set forth in SEQ ID NO: 97, a HCDR2 comprising an amino acid sequence as set forth in SEQ ID NO: 98, a HCDR3 comprising an amino acid sequence as set forth in SEQ ID NO: 99, a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a HCDR1 comprising an amino acid sequence as set forth in SEQ ID NO: 105, a HCDR2 comprising an amino acid sequence as set forth in SEQ ID NO: 106, a HCDR3 comprising an amino acid sequence as set forth in SEQ ID NO: 107, a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a HCDR1 comprising an amino acid sequence as set forth in SEQ ID NO: 113, a HCDR2 comprising an amino acid sequence as set forth in SEQ ID NO: 114, a HCDR3 comprising an amino acid sequence as set forth in SEQ ID NO: 115, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a HCDR1 comprising an amino acid sequence as set forth in SEQ ID NO: 121, a HCDR2 comprising an amino acid sequence as set forth in SEQ ID NO: 122, a HCDR3 comprising an amino acid sequence as set forth in SEQ ID NO: 123, a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a HCDR1 comprising an amino acid sequence as set forth in SEQ ID NO: 129, a HCDR2 comprising an amino acid sequence as set forth in SEQ ID NO: 130, a HCDR3 comprising an amino acid sequence as set forth in SEQ ID NO: 131, a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a HCDR1 comprising an amino acid sequence as set forth in SEQ ID NO: 137, a HCDR2 comprising an amino acid sequence as set forth in SEQ ID NO: 138, a HCDR3 comprising an amino acid sequence as set forth in SEQ ID NO: 139, a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142;
xvii. a HCDR1 comprising an amino acid sequence as set forth in SEQ ID NO: 145, a HCDR2 comprising an amino acid sequence as set forth in SEQ ID NO: 146, a HCDR3 comprising an amino acid sequence as set forth in SEQ ID NO: 147, a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150;
xviii. a HCDR1 comprising an amino acid sequence as set forth in SEQ ID NO: 153, a HCDR2 comprising an amino acid sequence as set forth in SEQ ID NO: 154, a HCDR3 comprising an amino acid sequence as set forth in SEQ ID NO: 155, a LCDR1 comprising an amino acid sequence as set forth in SEQ ID NO: 156, a LCDR2 comprising an amino acid sequence as set forth in SEQ ID NO: 157, and a LCDR3 comprising an amino acid sequence as set forth in SEQ ID NO: 158;
xix. a HCDR1 comprising an amino acid sequence as set forth in SEQ ID NO: 169, a HCDR2 comprising an amino acid sequence as set forth in SEQ ID NO: 170, a HCDR3 comprising an amino acid sequence as set forth in SEQ ID NO: 171, a LCDR1 comprising an amino acid sequence as set forth in SEQ ID NO: 172, a LCDR2 comprising an amino acid sequence as set forth in SEQ ID NO: 173, and a LCDR3 comprising an amino acid sequence as set forth in SEQ ID NO: 174;
xx. a HCDR1 comprising an amino acid sequence as set forth in SEQ ID NO: 177, a HCDR2 comprising an amino acid sequence as set forth in SEQ ID NO: 178, a HCDR3 comprising an amino acid sequence as set forth in SEQ ID NO: 179, a LCDR1 comprising an amino acid sequence as set forth in SEQ ID NO: 180, a LCDR2 comprising an amino acid sequence as set forth in SEQ ID NO: 181, and a LCDR3 comprising an amino acid sequence as set forth in SEQ ID NO: 182; or
xxi. a HCDR1 comprising an amino acid sequence as set forth in SEQ ID NO: 185, a HCDR2 comprising an amino acid sequence as set forth in SEQ ID NO: 186, a HCDR3 comprising an amino acid sequence as set forth in SEQ ID NO: 187, a LCDR1 comprising an amino acid sequence as set forth in SEQ ID NO: 188, a LCDR2 comprising an amino acid sequence as set forth in SEQ ID NO: 189, and a LCDR3 comprising an amino acid sequence as set forth in SEQ ID NO: 190.

In some embodiments, the antibody or antigen-binding fragment thereof of the present disclosure comprises a VH region having an amino acid sequence as set forth in SEQ ID NOs: 7, 15, 23, 31, 39, 47, 55, 63, 71, 79, 103, 111, 119, 127, 135, 143, 151, 159, 175, 183, or 191, or a homologous sequence thereof having at least 80% sequence identity to SEQ ID NOs: 7, 15, 23, 31, 39, 47, 55, 63, 71, 79, 103, 111, 119, 127, 135, 143, 151, 159, 175, 183, or 191.

In some embodiments, the antibody or antigen-binding fragment thereof of the present disclosure comprises a VL region having an amino acid sequence as set forth in SEQ ID NOs: 8, 16, 24, 32, 40, 48, 56, 64, 72, 80, 104, 112, 120, 128, 136, 144, 152, 160, 176, 184, or 192, or a homologous sequence thereof having at least 80% sequence identity to SEQ ID NOs: 8, 16, 24, 32, 40, 48, 56, 64, 72, 80, 104, 112, 120, 128, 136, 144, 152, 160, 176, 184, or 192.

In some embodiments, the antibody or antigen-binding fragment thereof of the present disclosure comprises a VH/VL amino acid sequence pair selected from the group consisting of SEQ ID NOs: 7/8, 15/16, 23/24, 31/32, 39/40, 47/48, 55/56, 63/64, 71/72, 79/80, 103/104, 111/112, 119/120, 127/128, 135/136, 143/144, 151/152, 159/160, 175/176, 183/184, or 191/192.

In some embodiments, the antibody or antigen-binding fragment thereof of the present disclosure further comprises one or more amino acid residue substitutions or modifications yet retains binding affinity to GPRC5D. In some embodiments, at least one of the substitutions or modifications is in one or more of the CDR sequences of the VH region or VL region. In some embodiments, at least one of the substitutions or modifications is in one or more of the non-CDR sequences of the VH region or VL region. In some embodiments, the antibody or antigen-binding fragment thereof of the present disclosure further comprises one or more non-natural amino acid (NNAA) substitution. In some embodiments, the NNAA is capable of being conjugated.

In some embodiments, the antibody or antigen-binding fragment thereof of the present disclosure has one or more binding properties selected from the group consisting of:
i. being with cross reactivity against human, cynomolgus and mouse GPRC5D;
ii. being capable of binding to human GPRC5D, cynomolgus GPRC5D, or mouse GPRC5D as measured by FACS assay;
iii. binding to human GPRC5D at an EC₅₀ of no more than 10 nM as measure as measured by FACS assay;
iv. showing T-cell dependent cytotoxicity effect as measured by FACS assay.

In another aspect, the present disclosure provides an antibody or antigen-binding fragment thereof, which competes for binding to GPRC5D with the antibody or antigen-binding fragment thereof as described above.

In some embodiments, the antibody or antigen-binding fragment thereof of the present disclosure is a chimeric, a humanized or a human antibody or an antigen-binding fragment thereof.

In some embodiments, the antibody or antigen-binding fragment thereof of the present disclosure is a labeled antibody, a bivalent antibody, an anti-idiotypic antibody or a fusion protein.

In some embodiments, the antibody or antigen-binding fragment thereof of the present disclosure is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

In some embodiments, the antibody or antigen-binding fragment thereof of the present disclosure further comprises an Fc region. In some embodiments, the Fe region is an Fc region of human immunoglobulin (Ig). In some embodiments, the Fc region is an Fc region of human IgG. In some embodiments, the Fc region is derived from human IgG1, IgG2, IgG3, or IgG4. In some embodiments, the Fc region comprises an amino acid sequence selected from the group consisting of SEQ ID NOs: 161-163.

In some embodiments, the light chain of the antibody or antigen-binding fragment thereof of the present disclosure is a λ light chain or a κ light chain.

In some embodiments, the antibody or antigen-binding fragment thereof of the present disclosure is a bispecific or multi-specific antibody or an antigen-binding fragment thereof. In some embodiments, the antibody or antigen-binding fragment thereof of the present disclosure is capable of specifically binding to one or more additional antigens other than GPRC5D, or a second epitope on GPRC5D. In some embodiments, the one or more additional antigens other than GPRC5D are selected from the group consisting of KRAS, ERK, XPO1, mTORC1/2, PAK4, NAMPT, ATR, EGFR, FGFR, VEGF, LILRB, c-MET, Her2, Her3, CTLA4, GITA, CD112R, CD2, CD3, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPα, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), BTLA, CD160, CTLA-4 (CD152), IDO1, IDO2, ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), and TGFβ.

In some embodiments, the antibody or antigen-binding fragment thereof of the present disclosure is a bispecific antibody or antigen-binding fragment thereof that binds to GPRC5D and CD3. In some embodiments, the bispecific antibody or antigen-binding fragment thereof comprises a GPRC5D binding moiety and a CD3 binding moiety. In some embodiments, the CD3 binding moiety comprises:
one or two or three heavy chain complementarity determining regions (HCDR1, HCDR2 and/or HCDR3) contained within the heavy chain variable (VH) region sequence of SEQ ID NO: 87 or 95; and/or
one or two or three light chain complementarity determining regions (LCDR1, LCDR2 and/or LCDR3) contained with the light chain variable (VL) region sequence of SEQ ID NO: 88 or 96.

In some embodiments, the CD3 binding moiety comprises:
a HCDR1 comprising an amino acid sequence as set forth in SEQ ID NO: 81 or 89, a HCDR2 comprising an amino acid sequence as set forth in SEQ ID NO: 82 or 90, and a HCDR3 comprising an amino acid sequence as set forth in SEQ ID NO: 83 or 91; and/or
a LCDR1 comprising an amino acid sequence as set forth in SEQ ID NO: 84 or 92, a LCDR2 comprising an amino acid sequence as set forth in SEQ ID NO: 85 or 93, and a LCDR3 comprising an amino acid sequence as set forth in SEQ ID NO: 86 or 94.

In some embodiments, the CD3 binding moiety comprises:
i. a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86; or
ii. a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94.

In some embodiments, the CD3 binding moiety comprises a VH/VL amino acid sequence pair of SEQ ID NOs: 87/88 or 95/96.

In some embodiments, the CD3 binding moiety further comprises a constant domain CL and a constant domain CH1. In some embodiments, the constant domains CL and CH1 are replaced by each other.

In some embodiments, the bispecific antibody or multi-specific antibody or antigen-binding fragment thereof of the present disclosure further comprises an Fc region. In some embodiments, the bispecific antibody or multi-specific antibody or antigen-binding fragment thereof of the present disclosure further comprises an Fc region of human immunoglobulin (Ig). In some embodiments, the bispecific antibody or multi-specific antibody or antigen-binding fragment thereof of the present disclosure further comprises an Fc region of human IgG. In some embodiments, the Fc region is derived from human IgG1, IgG2, IgG3, or IgG4. In some embodiments, the Fc region comprises one or more amino acid substitutions selected from the group consisting of: L234A, L235A, S354C, T366W, Y349C, T366S, L368A, and Y407V (according to EU numbering). In some embodiments, the Fc region comprises L234A and L235A (according to EU numbering) amino acid substitutions. In some embodiments, the Fc region of one heavy chain comprises S354C and T366W (according to EU numbering) amino acid substitutions, and the Fc region of the other heavy chain comprises Y349C, T366S, L368A, and Y407V (according to EU numbering) amino acid substitutions.

In some embodiments, the antibody or antigen-binding fragment thereof of the present disclosure is linked to one or more conjugate moieties. In some embodiments, the conjugate moiety comprises a clearance-modifying agent, a chemotherapeutic agent, a toxin, a radioactive isotope, a lanthanide, a detectable label, a DNA-alkylator, a topoisomerase inhibitor, a tubulin-binder, a purification moiety or other anticancer drugs. In some embodiments, the conjugate moiety is covalently attached either directly or via a linker.

In another aspect, the present disclosure provides a chimeric antigen receptor comprising the antibody or antigen-binding fragment thereof provided herein, a transmembrane region and an intracellular signal region. In some embodiments, the transmembrane region comprises a transmembrane region of CD3, CD4, CD8 or CD28. In some embodiments, the intracellular signal region is selected from the group consisting of: an intracellular signal regions sequence of CD3 (e.g. CD3ζ), FcγR1, CD27, CD28, CD137, CD134, MyD88, CD40, CD278, TLRs, or a combination thereof. In some embodiments, the antigen-binding fragment of the chimeric antigen receptor is a scFv. In some embodiments, the chimeric antigen receptor is grafted onto an allogeneic cell, an autologous cell or a xenogeneic cell. In some embodiments, the chimeric antigen receptor is grafted onto an immune effector cell. In some embodiments, the chimeric antigen receptor is grafted onto a T cell, a natural killer cell, a macrophage cell, or a tumor-infiltrating lymphocyte.

In another aspect, the present disclosure provides a pharmaceutical composition comprising the antibody or antigen-binding fragment thereof, and/or the chimeric antigen receptor of the present disclosure, and one or more pharmaceutically acceptable carriers.

In another aspect, the present disclosure provides an isolated polynucleotide encoding the antibody or antigen-binding fragment thereof and/or the chimeric antigen receptor of the present disclosure.

In another aspect, the present disclosure provides a vector comprising the isolated polynucleotide of the present disclosure.

In another aspect, the present disclosure provides a host expression system comprising the vector of the present disclosure or having the polynucleotide of the present disclosure integrated into genome thereof. In some embodiments, the host expression system of the present disclosure is a microorganism, a yeast, or a mammalian cell. In some embodiments, the microorganism is selected from the group consisting of E. coli and B. subtilis. In some embodiments, the yeast is Saccharomyces. In some embodiments, the mammalian cell is selected from the group consisting of COS, CHO-S, CHO-KI, HEK-293, and 3T3 cells.

In another aspect, the present disclosure provides a virus comprising the vector of the present disclosure.

In another aspect, the present disclosure provides a kit comprising the antibody or antigen-binding fragment thereof of the present disclosure and/or the chimeric antigen receptor of the present disclosure and/or the pharmaceutical composition of the present disclosure, and a second therapeutic agent.

In another aspect, the present disclosure provides a method of expressing the antibody or antigen-binding fragment thereof of the present disclosure and/or the chimeric antigen receptor of the present disclosure, comprising culturing the host expression system of the present disclosure under the condition at which the antibody or antigen-binding fragment of the present disclosure is expressed.

In another aspect, the present disclosure provides a method of treating, preventing or alleviating a disease, disorder or condition in a subject, comprising administering to the subject a therapeutically effective amount of the antibody or antigen-binding fragment thereof and/or the chimeric antigen receptor and/or the pharmaceutical composition of the present disclosure.

In another aspect, the present disclosure provides use of the antibody or antigen-binding fragment thereof and/or the chimeric antigen receptor and/or the pharmaceutical composition of the present disclosure in the manufacture of a medicament for treating a GPRC5D-related disease, disorder or condition in a subject.

In another aspect, the present disclosure provides use of the antibody or antigen-binding fragment thereof and/or the chimeric antigen receptor and/or the pharmaceutical composition of the present disclosure in the manufacture of a diagnostic reagent for diagnosing a GPRC5D-related disease, disorder or condition.

In some embodiments, the disease, disorder or condition is cancer. In some embodiments, the cancer is a solid tumor or hematologic tumor. In some embodiments, the disease, disorder or condition is a GPRC5D-expressing B cell cancer. In some embodiments, the disease, disorder or condition is selected from the group consisting of lung cancer (e.g., non-small-cell lung cancer (NSCLC), small cell lung cancer (SCLC), adenocarcinoma of the lung, or squamous cell carcinoma of the lung), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), cutaneous or intraocular melanoma, thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma. In some embodiments, the disease, disorder or condition is multiple mycloma.

In some embodiments, the subject is human.

In some embodiments, the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

In some embodiments, the method of treating, preventing or alleviating a disease, disorder or condition in a subject further includes administering to the subject in need thereof an additional therapeutic agent. In some embodiments, the additional therapeutic agent is selected from the group consisting of: an active agent, an imaging agent, a cytotoxic agent, and angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, and a microRNA. In some embodiments, the additional therapeutic agent is administered to the subject in need before, after or simultaneously with the antibody or antigen-binding fragment thereof and/or the pharmaceutical composition of the present disclosure.

In another aspect, the present disclosure provides a method of inactivating a GPRC5D-expressing cell in vivo or in vitro, comprising contacting the GPRC5D-expressing cell with the antibody or antigen-binding fragment thereof and/or the chimeric antigen receptor and/or the pharmaceutical composition of the present disclosure.

In another aspect, the present disclosure provides a method of modulating GPRC5D activity in a GPRC5D-expressing cell, comprising exposing the GPRC5D-expressing cell to the antibody or antigen-binding fragment thereof and/or the chimeric antigen receptor and/or the pharmaceutical composition of the present disclosure.

In another aspect, the present disclosure provides a method of detecting presence or amount of GPRC5D in a sample, comprising contacting the sample with the antibody or antigen-binding fragment thereof and/or the chimeric antigen receptor and/or the pharmaceutical composition of the present disclosure, and determining the presence or the amount of GPRC5D in the sample.

In another aspect, the present disclosure provides a method of diagnosing a GPRC5D-related disease, disorder or condition in a subject, comprising: a) obtaining a sample from the subject; b) contacting the sample obtained from the subject with the antibody or antigen-binding fragment thereof and/or the chimeric antigen receptor and/or the pharmaceutical composition of the present disclosure; c) determining presence or amount of GPRC5D in the sample; and d) correlating the presence or the amount of GPRC5D to existence or status of the GPRC5D-related disease, disorder or condition in the subject.

In another aspect, the present disclosure provides a kit comprising the antibody or antigen-binding fragment thereof and/or the chimeric antigen receptor and/or the pharmaceutical composition of the present disclosure, useful in detecting GPRC5D, optionally recombinant GPRC5D, GPRC5D expressed on cell surface, or GPRC5D-expressing cells.

### BRIEF DESCRIPTION OF THE DRAWINGS

FIG. 1 shows the binding affinity of the selected chimeric antibodies to human GPRC5D (FIG. 1A), cynomolgus GPRC5D (FIG. 1D) and mouse GPRC5D (FIG. 1C).

FIG. 2 shows the T-cell dependent cytotoxicity effect of selected bispecific hIgG-scFv antibodies on CHOS-hGPRC5D cells.

FIG. 3 shows the T-cell dependent cytotoxicity effect of selected bispecific Crossmab antibodies on MM.1S cells.

FIG. 4 shows the tumor growth curve showing the in vivo efficacy of selected bispecific Crossmab antibodies.

FIG. 5 shows the CAR-T cytotoxicity of selected clones.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

### Definitions

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain comprises a variable region (VH) and a first, second, third, and optionally fourth constant region (CH1, CH2, CH3, CH4 respectively); mammalian light chains are classified as λ or κ, while each light chain comprises a variable region (VL) and a constant region. The antibody has a “Y” shape, with the stem of the Y comprising the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more (e.g., 1, 2, 3, 4, 5, or 6) CDRs, or any other antibody fragment that binds to an antigen but does not comprise an intact native antibody structure. Examples of antigen-binding fragments include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multi-specific antibody, a camelized single domain antibody, a nanobody, a domain antibody, or a bivalent domain antibody. An antigen-binding fragment is capable of binding to the same antigen or epitope to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a linker (e.g., a peptide sequence) (Huston J S et al., Proc Natl Acad Sci USA, 85:5879 (1988)).

“Single-chain Fv-Fc antibody” or “scFv-Fc” refers to an engineered antibody consisting of a scFv connected to the Fc region of an antibody.

“Camelized single domain antibody”, “heavy chain antibody”, or “HCAb” refers to an antibody that contains two VH domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al., Immunogenetics. April; 54(1):39-47 (2002); Nguyen V K. et al., Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a VH domain connected to a VL domain in the same polypeptide chain (VH-VL or VL-VH) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more VH domains are covalently joined with a peptide linker to create a bivalent or multivalent domain antibody. The two VH domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or antigen-binding fragment having multiple antigen-binding sites. As such, the terms “bivalent”, “tetravalent”, and “hexavalent” denote the presence of two antigen-binding sites, four antigen-binding sites, and six antigen-binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

As used herein, a “multi-specific” antibody refers to an antibody that specifically binds to at least two distinct antigens or at least two distinct epitopes within the same antigen. Multi-specific antibody may bind for example two, three, four, five or more distinct antigens or distinct epitopes within the same antigen.

In certain embodiments, an “scFv dimer” is a bivalent diabody or bispecific scFv (BsFv) comprising VH-VL (linked by a peptide linker) dimerized with another VH-VL moiety such that VH's of one moiety coordinate with the VL's of the other moiety and form two binding sites which can target the same antigens (or epitopes) or different antigens (or epitopes). In other embodiments, an “scFv dimer” is a bispecific diabody comprising VH1-VL2 (linked by a peptide linker) associated with VL1-VH2 (also linked by a peptide linker) such that VH1 and VL1 coordinate and VH2 and VL2 coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two VH moieties linked by a peptide linker (e.g., a long flexible linker) and bound to two VL moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human. The CDRs of humanized antibodies provided in the present disclosure may contain mutation(s) compared to the CDRs of their parent antibodies.

The term “affinity” as used herein refers to the strength of non-covalent interaction between an immunoglobulin molecule (i.e., antibody) or antigen-binding fragment thereof and an antigen.

An antibody or antigen-binding fragment thereof that “specifically binds” or “specific binding” to a target (e.g., an epitope) is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. For example, an antibody that specifically binds to a GPRC5D epitope is an antibody that binds this GPRC5D epitope with greater affinity, avidity, more readily, and/or with greater duration than it binds to other GPRC5D epitopes or non-GPRC5D epitopes. It is also understood by reading this definition that, for example, an antibody (or moiety or epitope) that specifically binds to a first target may or may not specifically bind to a second target. As such, “specific binding” or “specifically bind” does not necessarily require (although it can include) exclusive binding. Generally, but not necessarily, reference to binding means specific binding.

The ability to “compete for binding to GPRC5D” as used herein refers to the ability of a first antibody or antigen-binding fragment to inhibit the binding interaction between GPRC5D and a second anti-GPRC5D antibody to any detectable degree. In certain embodiments, an antibody or antigen-binding fragment that competes for binding to GPRC5D inhibits the binding interaction between GPRC5D and a second anti-GPRC5D antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e., including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g., Cys, Ser, Thr, Asn and Gln), among amino acid residues with acidic side chains (e.g., Asp, Glu), among amino acid residues with basic side chains (e.g., His, Lys, and Arg), or among amino acid residues with aromatic side chains (e.g., Trp, Tyr, and Phe). As known in the art, conservative substitution usually does not cause significant change in the protein conformational structure, and therefore could retain the biological activity of a protein.

The term “homologous” as used herein refers to nucleic acid sequences (or its complementary strand) or amino acid sequences that have sequence identity of at least 60% (e.g., at least 65%, 70%, 75%, 80%, 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

“Antibody-dependent cell-mediated cytotoxicity” or “ADCC” as used herein refers to a cell-mediated reaction in which effector cells that express Fc receptors (FcRs) recognize bound antibody or antigen-binding fragment on a target cell and subsequently cause lysis of the target cell. “ADCC activity” or “ADCC effect” refers to the ability of the antibody or antigen-binding fragment which is bound on the target cell to elicit an ADCC reaction as described above.

“Complement dependent cytotoxicity” or “CDC” as used herein refers to a mechanism by which antibodies can mediate specific target cell lysis through activation of an organism's complement system. In CDC, the C1q binds the antibody and this binding triggers the complement cascade which leads to the formation of the membrane attack complex (MAC) (C5b to C9) at the surface of the target cell, as a result of the classical pathway complement activation. “CDC activity” or “CDC effect” refers to the ability of the antibody or antigen-binding fragment which is bound on the target cell to elicit a CDC reaction as described above.

“Target cells” as used herein refer to cells to which antibodies comprising an Fc region specifically bind, generally via the protein part that is C-terminal to the Fc region. “Effector cells” are leukocytes which express one or more Fc receptors and perform effector functions. Examples of human leukocytes which mediate ADCC include peripheral blood mononuclear cells (PBMCs), natural killer (NK) cells, monocytes, cytotoxic T cells and neutrophils; with PBMCs and NK cells being preferred. The effector cells may be isolated from a native source thereof, e.g., from blood or PBMCs as is known in the art.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated”, but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCl, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSGS, pXT1, pCDEF3, pSVSPORT, pEF-Bos etc.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient”, “subject” or “individual” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a GPRC5D related disease, or refer to identification of a subject with a GPRC5D related disease who may benefit from a particular treatment regimen. In some embodiments, diagnosis contains the identification of abnormal amount or activity of GPRC5D. In some embodiments, diagnosis refers to the identification of a cancer in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from stomach, heart, liver, spleen, lung, kidney, skin or blood vessels of the subject.

The term “GPRC5D” as used herein, refers to the G-protein coupled receptor family C group 5 member D, includes any variants, conformations, isoforms and species homologs of GPRC5D which are naturally expressed by cells or are expressed by cells transfected with the GPRC5D gene. For example, GPRC5D described herein may refer to the G-protein coupled receptor family C group 5 member D protein derived from any vertebrate source, including mammals such as primates (e.g., humans, monkeys) and rodents (e.g., mice and rats). Exemplary sequence of human GPRC5D protein is, for example as described in GenBank Accession No. BC069341, NCBI Reference Sequence: NP_061124.1 and UniProtKB/Swiss-Prot Accession No. Q9NZD1 (see also Brauner-Osborne, H. et al., 2001, Biochim. Biophys. Acta 1518, 237-248). The term “GPRC5D” as used herein is intended to encompass any form of GPRC5D, for example, 1) native unprocessed GPRC5D molecule, “full-length” GPRC5D chain or naturally occurring variants of GPRC5D, including, for example, splice variants or allelic variants; 2) any form of GPRC5D that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of GPRC5D subunit generated through recombinant method.

“CD3” as used herein, refers to the Cluster of Differentiation 3 protein and includes any variants, conformations, isoforms and species homologs of CD3 which are naturally expressed by cells or are expressed by cells transfected with the CD3 gene. For example, CD3 described herein may refer to the Cluster of Differentiation 3 protein derived from any vertebrate source, including mammals such as primates (e.g., humans, monkeys) and rodents (e.g., mice and rats). In mammals, the CD3 molecule is a multi-protein complex of six chains, including: a CD3gamma chain, a CD3delta chain, two CD3epsilon chains, and a homodimer of CD3zeta chains, wherein the CD3zeta chain is the intracellular tail of CD3 molecule, and the CD3gamma, CD3delta and CD3epsilon chains all contain extracellular domain (ECD) expressed on surface of T cells. Exemplary sequence of human CD3 includes human CD3epsilon protein (NCBI Ref Seq No. NP 000724), human CD3 delta protein (NCBI Ref Seq No. NP_000723), and human CD3gamma protein (NCBI Ref Seq No. NP_000064). Exemplary sequence of non-human CD3 includes Macaca fascicularis (monkey) CD3epsilon protein (NCBI Ref Seq No. NP_001270544), Macaca fascicularis (monkey) CD3delta protein (NCBI Ref Seq No. NP_001274617), Macaca fascicularis (monkey) CD3gamma protein (NCBI Ref Seq No. NP_001270839); Mus musculus (mouse) CD3epsilon protein (NCBI Ref Seq No. NP_031674), Mus musculus (mouse) CD3delta protein (NCBI Ref Seq No. NP_038515), Mus musculus domesticus (mouse) CD3gamma protein (NCBI Ref Seq No. AAA37400); Rattus norvegicus (Rat) CD3epsilon protein (NCBI Ref Seq No. NP_001101610), Rattus norvegicus (Rat) CD3delta protein (NCBI Ref Seq No. NP_037301), Rattus norvegicus (Rat) CD3gamma protein (NCBI Ref Seq No. NP_001071114). In certain embodiments, CD3 used herein can also be recombinant CD3, for example, including recombinant CD3epsilon protein, recombinant CD3delta protein, and recombinant CD3gamma protein, which may optionally be expressed as a recombinant CD3 complex. The recombinant CD3 complex may be expressed on a cell surface, or alternatively may be expressed as a soluble form which is not associated on a cell surface. In certain embodiments, the CD3 is human CD3. The terms “CD3”, “CD-3”, “CD3”, “cluster of differentiation 3” may be used interchangeably in the present disclosure.

The term “anti-GPRC5D antibody” refers to an antibody that binds to GPRC5D (e.g., human GPRC5D). The term “anti-human GPRC5D antibody” or “anti-hGPRC5D antibody” refers to an antibody that specifically binds to human GPRC5D.

The term “anti-CD3 antibody” refers to an antibody that specifically binds to CD3 (e.g., human CD3). The term “anti-human CD3 antibody” refers to an antibody that specifically binds to human CD3. In some embodiments, the anti-CD3 antibody provided herein specifically binds to a CD3gamma protein. In some embodiments, the anti-CD3 antibody provided herein specifically binds to a CD3delta protein. In some embodiments, the anti-CD3 antibody provided herein specifically binds to a CD3epsilon protein.

The term “CD3gamma” as used herein is intended to encompass any form of CD3gamma, for example, 1) native unprocessed CD3gamma molecule, “full-length” CD3gamma chain or naturally occurring variants of CD3gamma, including, for example, splice variants or allelic variants; 2) any form of CD3gamma that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3gamma subunit generated through recombinant method.

The term “CD3delta” as used herein is intended to encompass any form of CD3delta, for example, 1) native unprocessed CD3delta molecule, “full-length” CD3delta chain or naturally occurring variants of CD3delta, including, for example, splice variants or allelic variants; 2) any form of CD3delta that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3delta subunit generated through recombinant method.

The term “CD3epsilon” as used herein is intended to encompass any form of CD3epsilon, for example, 1) native unprocessed CD3epsilon molecule, “full-length” CD3epsilon chain or naturally occurring variants of CD3epsilon, including, for example, splice variants or allelic variants; 2) any form of CD3epsilon that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3epsilon subunit generated through recombinant method.

A “GPRC5D related” or “GPRC5D-related” disease, disorder or condition as used herein refers to any disease, disorder or condition caused by, exacerbated by, or otherwise linked to increased or decreased expression or activities of GPRC5D. In some embodiments, the GPRC5D-related disease, disorder or condition is a disorder related to excessive cell proliferation, such as, for example, cancer. In certain embodiments, the GPRC5D-related disease or condition is characterized in expressing or over-expressing of GPRC5D and/or GPRC5D related genes.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “GPRC5D-expressing cell” as used herein refer to a cell that expresses GPRC5D on the surface of the cell.

### Anti-GPRC5D Antibodies

The present disclosure provides anti-GPRC5D antibodies and antigen-binding fragments thereof. The anti-GPRC5D antibodies and antigen-binding fragments provided herein are capable of binding (e.g., specifically binding) to GPRC5D (e.g., human GPRC5D).

Binding affinity of the antibody or antigen-binding fragment thereof provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g., K_{D}) can be appropriately determined using suitable methods known in the art, including, for example, flow cytometry assay. In some embodiments, binding of the antibody or antigen-binding fragment thereof to the antigen at different concentrations can be determined by flow cytometry, the determined mean fluorescence intensity (MFI) can be firstly plotted against antibody concentration, K_{D} value can then be calculated by fitting the dependence of specific binding fluorescence intensity (Y) and the concentration of antibodies (X) into the one site saturation equation: Y=Bₘₐₓ*X/(K_{D}+X) using Prism version 5 (GraphPad Software, San Diego, CA), wherein Bₘₐₓ refers to the maximum specific binding of the tested antibody to the antigen.

Binding of the antibodies or the antigen-binding fragments thereof provided herein to GPRC5D can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), FACS assay, and other binding assays.

In certain embodiments, the antibodies or antigen-binding fragments thereof provided herein are capable of binding to human GPRC5D, cynomolgus GPRC5D or mouse GPRC5D as measured by FACS assay. In certain embodiments, the antibodies or antigen-binding fragments thereof provided herein are with cross reactivity against human, cynomolgus and mouse GPRC5D.

The term “cross reactivity” as used herein refers to the ability of a binding protein to bind a target other than that against which it was raised. Generally, a binding protein will bind its target tissue(s)/antigen(s) with an appropriately high affinity, but will display an appropriately low affinity for non-target normal tissues/antigens. Individual binding proteins are generally selected to meet two criteria: (1) antibody binding, as visualized using staining methods known in the art, to tissue appropriate for the known expression of the antibody target and (2) a similar staining pattern between human and tox species (e.g., mouse and cynomolgus monkey) tissues from the same organ. These and other methods of assessing cross-reactivity are known to one skilled in the art (for example, US20090311253A1).

In certain embodiments, the antibodies or antigen-binding fragments thereof provided herein bind to human GPRC5D at an EC₅₀ of no more than 10 nM (e.g., no more than 9 nM, no more than 8 nM, no more than 7 nM, no more than 6 nM, no more than 5 nM, no more than 4 nM, no more than 3 nM, no more than 2 nM, no more than 1 nM) as measured by FACS assay. In certain embodiments, the antibodies or antigen-binding fragments thereof provided herein bind to cynomolgus GPRC5D and/or mouse GPRC5D at an EC₅₀ of no more than 20 nM (e.g., no more than 15 nM, no more than 10 nM, no more than 9 nM, no more than 8 nM, no more than 7 nM, no more than 6 nM, no more than 5 nM, no more than 4 nM, no more than 3 nM, no more than 2 nM, no more than 1 nM) as measured by FACS assay.

In certain embodiments, the antibodies or antigen-binding fragments thereof provided herein show T-cell dependent cytotoxicity effect (e.g., as measured by FACS assay). In certain embodiments, the T-cell dependent cytotoxicity effect is measured by the method as described in Example 3 of the present disclosure.

### Illustrative Anti-GPRC5D Antibodies

In certain embodiments, the present disclosure provides antibodies or antigen-binding fragments thereof which bind to GPRC5D, comprising:
one or two or three heavy chain complementarity determining regions (HCDR1, HCDR2 and/or HCDR3) contained within any one of the heavy chain variable (VH) region sequences selected from the group consisting of SEQ ID NOs: 7, 15, 23, 31, 39, 47, 55, 63, 71, 79, 103, 111, 119, 127, 135, 143, 151, 159, 175, 183, and 191; and/or
one or two or three light chain complementarity determining regions (LCDR1, LCDR2 and LCDR3) contained within any one of the light chain variable (VL) region sequences selected from the group consisting of SEQ ID NOs: 8, 16, 24, 32, 40, 48, 56, 64, 72, 80, 104, 112, 120, 128, 136, 144, 152, 160, 176, 184, and 192.

A person skilled in the art can define or identify CDR boundaries of a VH or VL region by well-known methods in the art as long as the amino acid sequence of the VH or VL region is known. For example, CDR boundaries for an antibody or antigen-binding fragment thereof may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et at, Sequences of Proteins of immunological Interest, 5th Ed. Public Health Service, National Institutes of Health, Bethesda, Md. (1991); Marie-Paule Lefranc et al, Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). In some embodiments, the CDR boundaries of the antibodies or antigen-binding fragments thereof provided herein are identified by the convention of Kabat. In some embodiments, the CDR boundaries of the antibodies or antigen-binding fragments thereof provided herein are identified by the convention of IMGT. In some embodiments, the CDR boundaries of the antibodies or antigen-binding fragments thereof provided herein are identified by the convention of Chothia. In some embodiments, the CDR boundaries of the antibodies or antigen-binding fragments thereof provided herein are identified by the convention of Al-Lazikani.

In certain embodiments, the present disclosure provides antibodies or antigen-binding fragments thereof which bind to GPRC5D comprising one or more (e.g., 1, 2, 3, 4, 5, or 6) CDR sequences of an anti-GPRC5D antibody 36A3G8, 28H9C12, 26E2H6, 20C4E1, 15D6G2, 12G12C2, 71F8H2, 64G9C1, 98A3A8, 96C9E7, 90G6H3, 89F11E3, 87A3G8, 83A9B8, 82A1C10, 119E4B4, 116G1G4, 116F1G6, 110B4H5, 108C4C12 or 101B3G3.

Antibody “36A3G8” as used herein refers to a mouse monoclonal antibody comprising a heavy chain variable region having the sequence of SEQ ID NO: 7, and a light chain variable region having the sequence of SEQ ID NO: 8.

Antibody “28H9C12” as used herein refers to a mouse monoclonal antibody comprising a heavy chain variable region having the sequence of SEQ ID NO: 15, and a light chain variable region having the sequence of SEQ ID NO: 16.

Antibody “26E2H6” as used herein refers to a mouse monoclonal antibody comprising a heavy chain variable region having the sequence of SEQ ID NO: 23, and a light chain variable region having the sequence of SEQ ID NO: 24.

Antibody “20C4E1” as used herein refers to a mouse monoclonal antibody comprising a heavy chain variable region having the sequence of SEQ ID NO: 31, and a light chain variable region having the sequence of SEQ ID NO: 32.

Antibody “15D6G2” as used herein refers to a mouse monoclonal antibody comprising a heavy chain variable region having the sequence of SEQ ID NO: 39, and a light chain variable region having the sequence of SEQ ID NO: 40.

Antibody “12G12C2” as used herein refers to a mouse monoclonal antibody comprising a heavy chain variable region having the sequence of SEQ ID NO: 47, and a light chain variable region having the sequence of SEQ ID NO: 48.

Antibody “71F8H2” as used herein refers to a mouse monoclonal antibody comprising a heavy chain variable region having the sequence of SEQ ID NO: 55, and a light chain variable region having the sequence of SEQ ID NO: 56.

Antibody “64G9C1” as used herein refers to a mouse monoclonal antibody comprising a heavy chain variable region having the sequence of SEQ ID NO: 63, and a light chain variable region having the sequence of SEQ ID NO: 64.

Antibody “98A3A8” as used herein refers to a mouse monoclonal antibody comprising a heavy chain variable region having the sequence of SEQ ID NO: 71, and a light chain variable region having the sequence of SEQ ID NO: 72.

Antibody “96C9E7” as used herein refers to a mouse monoclonal antibody comprising a heavy chain variable region having the sequence of SEQ ID NO: 79, and a light chain variable region having the sequence of SEQ ID NO: 80.

Antibody “90G6H3” as used herein refers to a mouse monoclonal antibody comprising a heavy chain variable region having the sequence of SEQ ID NO: 103, and a light chain variable region having the sequence of SEQ ID NO: 104.

Antibody “89F11E3” as used herein refers to a mouse monoclonal antibody comprising a heavy chain variable region having the sequence of SEQ ID NO: 111, and a light chain variable region having the sequence of SEQ ID NO: 112.

Antibody “87A3G8” as used herein refers to a mouse monoclonal antibody comprising a heavy chain variable region having the sequence of SEQ ID NO: 119, and a light chain variable region having the sequence of SEQ ID NO: 120.

Antibody “83A9B8” as used herein refers to a mouse monoclonal antibody comprising a heavy chain variable region having the sequence of SEQ ID NO: 127, and a light chain variable region having the sequence of SEQ ID NO: 128.

Antibody “82A1C10” as used herein refers to a mouse monoclonal antibody comprising a heavy chain variable region having the sequence of SEQ ID NO: 135, and a light chain variable region having the sequence of SEQ ID NO: 136.

Antibody “119E4B4” as used herein refers to a mouse monoclonal antibody comprising a heavy chain variable region having the sequence of SEQ ID NO: 143, and a light chain variable region having the sequence of SEQ ID NO: 144.

Antibody “116GIG4” as used herein refers to a mouse monoclonal antibody comprising a heavy chain variable region having the sequence of SEQ ID NO: 151, and a light chain variable region having the sequence of SEQ ID NO: 152.

Antibody “116F1G6” as used herein refers to a mouse monoclonal antibody comprising a heavy chain variable region having the sequence of SEQ ID NO: 159, and a light chain variable region having the sequence of SEQ ID NO: 160.

Antibody “110B4H5” as used herein refers to a mouse monoclonal antibody comprising a heavy chain variable region having the sequence of SEQ ID NO: 175, and a light chain variable region having the sequence of SEQ ID NO: 176.

Antibody “108C4C12” as used herein refers to a mouse monoclonal antibody comprising a heavy chain variable region having the sequence of SEQ ID NO: 183, and a light chain variable region having the sequence of SEQ ID NO: 184.

Antibody “101B3G3” as used herein refers to a mouse monoclonal antibody comprising a heavy chain variable region having the sequence of SEQ ID NO: 191, and a light chain variable region having the sequence of SEQ ID NO: 192.

The specific amino acid sequences of the heavy chain variable region and light chain variable region of each exemplary antibody as described above are shown in Table 3 below.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 7, and three light chain CDRs (LCDR1, LCDR2 and LCDR3) contained within the VL region sequence as set forth in SEQ ID NO: 8.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 15, and three light chain CDRs (LCDR1, LCDR2 and LCDR3) contained within the VL region sequence as set forth in SEQ ID NO: 16.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 23, and three light chain CDRs (LCDR1, LCDR2 and LCDR3) contained within the VL region sequence as set forth in SEQ ID NO: 24.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 31, and three light chain CDRs (LCDR1, LCDR2 and LCDR3) contained within the VL region sequence as set forth in SEQ ID NO: 32.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 39, and three light chain CDRs (LCDR1, LCDR2 and LCDR3) contained within the VL region sequence as set forth in SEQ ID NO: 40.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 47, and three light chain CDRs (LCDR1, LCDR2 and LCDR3) contained within the VL region sequence as set forth in SEQ ID NO: 48.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 55, and three light chain CDRs (LCDR1, LCDR2 and LCDR3) contained within the VL region sequence as set forth in SEQ ID NO: 56.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 63, and three light chain CDRs (LCDR1, LCDR2 and LCDR3) contained within the VL region sequence as set forth in SEQ ID NO: 64.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 71, and three light chain CDRs (LCDR1, LCDR2 and LCDR3) contained within the VL region sequence as set forth in SEQ ID NO: 72.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 79, and three light chain CDRs (LCDR1, LCDR2 and LCDR3) contained within the VL region sequence as set forth in SEQ ID NO: 80.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 103, and three light chain CDRs (LCDR1, LCDR2 and LCDR3) contained within the VL region sequence as set forth in SEQ ID NO: 104.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 111, and three light chain CDRs (LCDR1, LCDR2 and LCDR3) contained within the VL region sequence as set forth in SEQ ID NO: 112.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 119, and three light chain CDRs (LCDR1, LCDR2 and LCDR3) contained within the VL region sequence as set forth in SEQ ID NO: 120.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 127, and three light chain CDRs (LCDR1, LCDR2 and LCDR3) contained within the VL region sequence as set forth in SEQ ID NO: 128.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 135, and three light chain CDRs (LCDR1, LCDR2 and LCDR3) contained within the VL region sequence as set forth in SEQ ID NO: 136.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 143, and three light chain CDRs (LCDR1, LCDR2 and LCDR3) contained within the VL region sequence as set forth in SEQ ID NO: 144.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 151, and three light chain CDRs (LCDR1, LCDR2 and LCDR3) contained within the VL region sequence as set forth in SEQ ID NO: 152.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 159, and three light chain CDRs (LCDR1, LCDR2 and LCDR3) contained within the VL region sequence as set forth in SEQ ID NO: 160.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 175, and three light chain CDRs (LCDR1, LCDR2 and LCDR3) contained within the VL region sequence as set forth in SEQ ID NO: 176.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 183, and three light chain CDRs (LCDR1, LCDR2 and LCDR3) contained within the VL region sequence as set forth in SEQ ID NO: 184.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 191, and three light chain CDRs (LCDR1, LCDR2 and LCDR3) contained within the VL region sequence as set forth in SEQ ID NO: 192.

In certain embodiments, the antibodies or antigen-binding fragments thereof provided herein comprise at least one (e.g., 1, 2, or 3) heavy or light chain CDR comprising an amino acid sequence selected from the group consisting of SEQ ID NOs: 1, 2, 3, 4, 5, 6, 9, 10, 11, 12, 13, 14, 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 97, 98, 99, 100, 101, 102, 105, 106, 107, 108, 109, 110, 113, 114, 115, 116, 117, 118, 121, 122, 123, 124, 125, 126, 129, 130, 131, 132, 133, 134, 137, 138, 139, 140, 141, 142, 145, 146, 147, 148, 149, 150, 153, 154, 155, 156, 157, 158, 169, 170, 171, 172, 173, 174, 177, 178, 179, 180, 181, 182, 185, 186, 187, 188, 189, and 190.

In certain embodiments, the antibodies or antigen-binding fragments thereof provided herein comprise a VH region comprising one or two or three of HCDR1, HCDR2 and HCDR3 comprising an amino acid sequence selected from the group consisting of SEQ ID NOs: 1, 2, 3, 9, 10, 11, 17, 18, 19, 25, 26, 27, 33, 34, 35, 41, 42, 43, 49, 50, 51, 57, 58, 59, 65, 66, 67, 73, 74, 75, 97, 98, 99, 105, 106, 107, 113, 114, 115, 121, 122, 123, 129, 130, 131, 137, 138, 139, 145, 146, 147, 153, 154, 155, 169, 170, 171, 177, 178, 179, 185, 186, and 187.

In certain embodiments, the antibodies or antigen-binding fragments thereof provided herein comprise a VL region comprising one or two or three of LCDR1, LCDR2 and LCDR3 comprising an amino acid sequence selected from the group consisting of SEQ ID NOs: 4, 5, 6, 12, 13, 14, 20, 21, 22, 28, 29, 30, 36, 37, 38, 44, 45, 46, 52, 53, 54, 60, 61, 62, 68, 69, 70, 76, 77, 78, 100, 101, 102, 108, 109, 110, 116, 117, 118, 124, 125, 126, 132, 133, 134, 140, 141,142, 148, 149, 150, 156, 157, 158, 172, 173, 174, 180, 181, 182, 188, 189, and 190.

In certain embodiments, the antibodies or antigen-binding fragments thereof provided herein comprise a HCDR1 comprising an amino acid sequence selected from the group consisting of SEQ ID NOs: 1, 9, 17, 25, 33, 41, 49, 57, 65, 73, 97, 105, 113, 121, 129, 137, 145, 153, 169, 177, and 185; a HCDR2 comprising an amino acid sequence selected from the group consisting of SEQ ID NOs: 2, 10, 18, 26, 34, 42, 50, 58, 66, 74, 98, 106, 114, 122, 130, 138, 146, 154, 170, 178, and 186; and a HCDR3 comprising an amino acid sequence selected from the group consisting of SEQ ID NOs: 3, 11, 19, 27, 35, 43, 51, 59, 67, 75, 99, 107, 115, 123, 131, 139, 147, 155, 171, 179, and 187.

In certain embodiments, the antibodies or antigen-binding fragments thereof provided herein comprise a LCDR1 comprising an amino acid sequence selected from the group consisting of SEQ ID NOs: 4, 12, 20, 28, 36, 44, 52, 60, 68, 76, 100, 108, 116, 124, 132, 140, 148, 156, 172, 180, and 188; a LCDR2 comprising an amino acid sequence selected from the group consisting of SEQ ID NOs: 5, 13, 21, 29, 37, 45, 53, 61, 69, 77, 101, 109, 117, 125, 133, 141, 149, 157, 173, 181, and 189; and a LCDR3 comprising an amino acid sequence selected from the group consisting of SEQ ID NOs: 6, 14, 22, 30, 38, 46, 54, 62, 70, 78, 102, 110, 118, 126, 134, 142, 150, 158, 174, 182, and 190.

In certain embodiments, the antibodies or antigen-binding fragments thereof provided herein comprise:
i. a HCDR1 comprising an amino acid sequence as set forth in SEQ ID NO: 1, a HCDR2 comprising an amino acid sequence as set forth in SEQ ID NO: 2, and a HCDR3 comprising an amino acid sequence as set forth in SEQ ID NO: 3;
ii. a HCDR1 comprising an amino acid sequence as set forth in SEQ ID NO: 9, a HCDR2 comprising an amino acid sequence as set forth in SEQ ID NO: 10, and a HCDR3 comprising an amino acid sequence as set forth in SEQ ID NO: 11;
iii. a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
iv. a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
v. a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
vi. a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
vii. a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
viii. a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
ix. a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
x. a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
xi. a HCDR1 comprising an amino acid sequence as set forth in SEQ ID NO: 97, a HCDR2 comprising an amino acid sequence as set forth in SEQ ID NO: 98, and a HCDR3 comprising an amino acid sequence as set forth in SEQ ID NO: 99;
xii. a HCDR1 comprising an amino acid sequence as set forth in SEQ ID NO: 105, a HCDR2 comprising an amino acid sequence as set forth in SEQ ID NO: 106, and a HCDR3 comprising an amino acid sequence as set forth in SEQ ID NO: 107;
xiii. a HCDR1 comprising an amino acid sequence as set forth in SEQ ID NO: 113, a HCDR2 comprising an amino acid sequence as set forth in SEQ ID NO: 114, and a HCDR3 comprising an amino acid sequence as set forth in SEQ ID NO: 115;
xiv. a HCDR1 comprising an amino acid sequence as set forth in SEQ ID NO: 121, a HCDR2 comprising an amino acid sequence as set forth in SEQ ID NO: 122, and a HCDR3 comprising an amino acid sequence as set forth in SEQ ID NO: 123;
xv. a HCDR1 comprising an amino acid sequence as set forth in SEQ ID NO: 129, a HCDR2 comprising an amino acid sequence as set forth in SEQ ID NO: 130, and a HCDR3 comprising an amino acid sequence as set forth in SEQ ID NO: 131;
xvi. a HCDR1 comprising an amino acid sequence as set forth in SEQ ID NO: 137, a HCDR2 comprising an amino acid sequence as set forth in SEQ ID NO: 138, and a HCDR3 comprising an amino acid sequence as set forth in SEQ ID NO: 139;
xvii. a HCDR1 comprising an amino acid sequence as set forth in SEQ ID NO: 145, a HCDR2 comprising an amino acid sequence as set forth in SEQ ID NO: 146, and a HCDR3 comprising an amino acid sequence as set forth in SEQ ID NO: 147;
xviii. a HCDR1 comprising an amino acid sequence as set forth in SEQ ID NO: 153, a HCDR2 comprising an amino acid sequence as set forth in SEQ ID NO: 154, and a HCDR3 comprising an amino acid sequence as set forth in SEQ ID NO: 155;
xix. a HCDR1 comprising an amino acid sequence as set forth in SEQ ID NO: 169, a HCDR2 comprising an amino acid sequence as set forth in SEQ ID NO: 170, and a HCDR3 comprising an amino acid sequence as set forth in SEQ ID NO: 171;
xx. a HCDR1 comprising an amino acid sequence as set forth in SEQ ID NO: 177, a HCDR2 comprising an amino acid sequence as set forth in SEQ ID NO: 178, and a HCDR3 comprising an amino acid sequence as set forth in SEQ ID NO: 179; or
xxi. a HCDR1 comprising an amino acid sequence as set forth in SEQ ID NO: 185, a HCDR2 comprising an amino acid sequence as set forth in SEQ ID NO: 186, and a HCDR3 comprising an amino acid sequence as set forth in SEQ ID NO: 187.

In certain embodiments, the antibodies or antigen-binding fragments thereof provided herein comprise:
i. a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
ii. a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
iii. a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
iv. a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
v. a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
vi. a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vii. a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
viii. a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
ix. a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
x. a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
xi. a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142;
xvii. a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150;
xviii. a LCDR1 comprising an amino acid sequence as set forth in SEQ ID NO: 156, a LCDR2 comprising an amino acid sequence as set forth in SEQ ID NO: 157, and a LCDR3 comprising an amino acid sequence as set forth in SEQ ID NO: 158;
xix. a LCDR1 comprising an amino acid sequence as set forth in SEQ ID NO: 172, a LCDR2 comprising an amino acid sequence as set forth in SEQ ID NO: 173, and a LCDR3 comprising an amino acid sequence as set forth in SEQ ID NO: 174;
xx. a LCDR1 comprising an amino acid sequence as set forth in SEQ ID NO: 180, a LCDR2 comprising an amino acid sequence as set forth in SEQ ID NO: 181, and a LCDR3 comprising an amino acid sequence as set forth in SEQ ID NO: 182; or
xxi. a LCDR1 comprising an amino acid sequence as set forth in SEQ ID NO: 188, a LCDR2 comprising an amino acid sequence as set forth in SEQ ID NO: 189, and a LCDR3 comprising an amino acid sequence as set forth in SEQ ID NO: 190.

In certain embodiments, the antibodies or antigen-binding fragments thereof provided herein comprise:
i. a HCDR1 comprising an amino acid sequence as set forth in SEQ ID NO: 1, a HCDR2 comprising an amino acid sequence as set forth in SEQ ID NO: 2, a HCDR3 comprising an amino acid sequence as set forth in SEQ ID NO: 3, a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
ii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
iii. a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
iv. a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
v. a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
vi. a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vii. a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
viii. a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
ix. a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
x. a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
xi. a HCDR1 comprising an amino acid sequence as set forth in SEQ ID NO: 97, a HCDR2 comprising an amino acid sequence as set forth in SEQ ID NO: 98, a HCDR3 comprising an amino acid sequence as set forth in SEQ ID NO: 99, a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a HCDR1 comprising an amino acid sequence as set forth in SEQ ID NO: 105, a HCDR2 comprising an amino acid sequence as set forth in SEQ ID NO: 106, a HCDR3 comprising an amino acid sequence as set forth in SEQ ID NO: 107, a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a HCDR1 comprising an amino acid sequence as set forth in SEQ ID NO: 113, a HCDR2 comprising an amino acid sequence as set forth in SEQ ID NO: 114, a HCDR3 comprising an amino acid sequence as set forth in SEQ ID NO: 115, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a HCDR1 comprising an amino acid sequence as set forth in SEQ ID NO: 121, a HCDR2 comprising an amino acid sequence as set forth in SEQ ID NO: 122, a HCDR3 comprising an amino acid sequence as set forth in SEQ ID NO: 123, a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a HCDR1 comprising an amino acid sequence as set forth in SEQ ID NO: 129, a HCDR2 comprising an amino acid sequence as set forth in SEQ ID NO: 130, a HCDR3 comprising an amino acid sequence as set forth in SEQ ID NO: 131, a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a HCDR1 comprising an amino acid sequence as set forth in SEQ ID NO: 137, a HCDR2 comprising an amino acid sequence as set forth in SEQ ID NO: 138, a HCDR3 comprising an amino acid sequence as set forth in SEQ ID NO: 139, a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142;
xvii. a HCDR1 comprising an amino acid sequence as set forth in SEQ ID NO: 145, a HCDR2 comprising an amino acid sequence as set forth in SEQ ID NO: 146, a HCDR3 comprising an amino acid sequence as set forth in SEQ ID NO: 147, a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150;
xviii. a HCDR1 comprising an amino acid sequence as set forth in SEQ ID NO: 153, a HCDR2 comprising an amino acid sequence as set forth in SEQ ID NO: 154, a HCDR3 comprising an amino acid sequence as set forth in SEQ ID NO: 155, a LCDR1 comprising an amino acid sequence as set forth in SEQ ID NO: 156, a LCDR2 comprising an amino acid sequence as set forth in SEQ ID NO: 157, and a LCDR3 comprising an amino acid sequence as set forth in SEQ ID NO: 158;
xix. a HCDR1 comprising an amino acid sequence as set forth in SEQ ID NO: 169, a HCDR2 comprising an amino acid sequence as set forth in SEQ ID NO: 170, a HCDR3 comprising an amino acid sequence as set forth in SEQ ID NO: 171, a LCDR1 comprising an amino acid sequence as set forth in SEQ ID NO: 172, a LCDR2 comprising an amino acid sequence as set forth in SEQ ID NO: 173, and a LCDR3 comprising an amino acid sequence as set forth in SEQ ID NO: 174;
xx. a HCDR1 comprising an amino acid sequence as set forth in SEQ ID NO: 177, a HCDR2 comprising an amino acid sequence as set forth in SEQ ID NO: 178, a HCDR3 comprising an amino acid sequence as set forth in SEQ ID NO: 179, a LCDR1 comprising an amino acid sequence as set forth in SEQ ID NO: 180, a LCDR2 comprising an amino acid sequence as set forth in SEQ ID NO: 181, and a LCDR3 comprising an amino acid sequence as set forth in SEQ ID NO: 182; or
xxi. a HCDR1 comprising an amino acid sequence as set forth in SEQ ID NO: 185, a HCDR2 comprising an amino acid sequence as set forth in SEQ ID NO: 186, a HCDR3 comprising an amino acid sequence as set forth in SEQ ID NO: 187, a LCDR1 comprising an amino acid sequence as set forth in SEQ ID NO: 188, a LCDR2 comprising an amino acid sequence as set forth in SEQ ID NO: 189, and a LCDR3 comprising an amino acid sequence as set forth in SEQ ID NO: 190.

The SEQ ID NOs of the heavy chain (denoted as “H”) variable region, light chain (denoted as “L”) variable region, HCDRs and LCDRs of each of the 21 monoclonal antibodies described above are shown in Table 1 below. The amino acid sequences of each CDR of the 21 exemplary monoclonal antibodies are shown in Table 2 below. Unless otherwise indicated, the CDR boundaries as described in Table 2 below were defined or identified by the convention of Kabat. The amino acid sequences of each VH and VL of the 21 exemplary monoclonal antibodies are shown in Table 3 below.

TABLE 1
SEQ ID NOs of VH, VL, HCDRs and LCDRs
of 21 exemplary monoclonal antibodies.

| | | Variable | | | |
|---|---|---|---|---|---|
| | | Region | CDR1 | CDR2 | CDR3 |
| Antibody | Chain | (SEQ ID NO) | (SEQ ID NO) | (SEQ ID NO) | (SEQ ID NO) |
| 36A3G8 | H | 7 | 1 | 2 | 3 |
| | L | 8 | 4 | 5 | 6 |
| 28H9C12 | H | 15 | 9 | 10 | 11 |
| | L | 16 | 12 | 13 | 14 |
| 26E2H6 | H | 23 | 17 | 18 | 19 |
| | L | 24 | 20 | 21 | 22 |
| 20C4E1 | H | 31 | 25 | 26 | 27 |
| | L | 32 | 28 | 29 | 30 |
| 15D6G2 | H | 39 | 33 | 34 | 35 |
| | L | 40 | 36 | 37 | 38 |
| 12G12C2 | H | 47 | 41 | 42 | 43 |
| | I | 48 | 44 | 45 | 46 |
| 71F8H2 | H | 55 | 49 | 50 | 51 |
| | L | 56 | 52 | 53 | 54 |
| 64G9C1 | H | 63 | 57 | 58 | 59 |
| | L | 64 | 60 | 61 | 62 |
| 98A3A8 | H | 71 | 65 | 66 | 67 |
| | L | 72 | 68 | 69 | 70 |
| 96C9E7 | H | 79 | 73 | 74 | 75 |
| | L | 80 | 76 | 77 | 78 |
| 90G6H3 | H | 103 | 97 | 98 | 99 |
| | L | 104 | 100 | 101 | 102 |
| 89F11E3 | H | 111 | 105 | 106 | 107 |
| | L | 112 | 108 | 109 | 110 |
| 87A3G8 | H | 119 | 113 | 114 | 115 |
| | L | 120 | 116 | 117 | 118 |
| 83A9B8 | H | 127 | 121 | 122 | 123 |
| | L | 128 | 124 | 125 | 126 |
| 82A1C10 | H | 135 | 129 | 130 | 131 |
| | L | 136 | 132 | 133 | 134 |
| 119E4B4 | H | 143 | 137 | 138 | 139 |
| | L | 144 | 140 | 141 | 142 |
| 116G1G4 | H | 151 | 145 | 146 | 147 |
| | L | 152 | 148 | 149 | 150 |
| 116F1G6 | H | 159 | 153 | 154 | 155 |
| | L | 160 | 156 | 157 | 158 |
| 110B4H5 | H | 175 | 169 | 170 | 171 |
| | L | 176 | 172 | 173 | 174 |
| 108C4C12 | H | 183 | 177 | 178 | 179 |
| | L | 184 | 180 | 181 | 182 |
| 101B3G3 | H | 191 | 185 | 186 | 187 |
| | L | 192 | 188 | 189 | 190 |

TABLE 2
Amino acid sequences of each CDR of 21
exemplary monoclonal antibodies.

| Description | SEQ ID NO | Amino Acid Sequence |
|---|---|---|
| HCDR1 | 1 | TYTMH |
| | 9 | DYGMH |
| | 17 | SYGMS |
| | 25 | DYGMH |
| | 33 | SHGMS |
| | 41 | TYGLS |
| | 49 | SYAMS |
| | 57 | SYAMS |
| | 65 | YYVTH |
| | 73 | YYILH |
| | 97 | SYIMH |
| | 105 | SSTIH |
| | 113 | SYSVY |
| | 121 | NYVIH |
| | 129 | NYAVT |
| | 137 | DTYMH |
| | 145 | YYIMH |
| | 153 | TYVMH |
| | 169 | FYIMH |
| | 177 | SYVIH |
| | 185 | GYTMN |
| HCDR2 | 2 | FINPSSGYTKYNQNFKD |
| | 10 | YISSGSSTIYYADTVKG |
| | 18 | TISSGGSYTYYPDSVKG |
| | 26 | FISSVSFTIYYADTVKG |
| | 34 | TISSGGRNTYYPDSVKG |
| | 42 | WINTFSGVPIYADDFKG |
| | 50 | TISDGGSYTYYPDNVKG |
| | 58 | TISDGGSYTYYPDNVKG |
| | 66 | YINPYNDGTMFNEKFKG |
| | 74 | YFNPYNDGNKFNERFRG |
| | 98 | YIYPGNGGTNYNQKFKD |
| | 106 | YIVPSTGYTNYNQKFKD |
| | 114 | VIWAGGSTNYNSALMS |
| | 122 | YFNPYNDGSKYNEKFKG |
| | 130 | VIWGDGSTYYHSALVS |
| | 138 | RIDPANGNTKYDPKFQG |
| | 146 | YINPYNDGAYYSARFKG |
| | 154 | YINPYNDGTKYNEKFKG |
| | 170 | YINPYNDGTKYNAKFKG |
| | 178 | YFNPYNDGTKYNEEFKG |
| | 186 | LINPYNGGTRYNQKFKD |
| HCDR3 | 3 | PYGNIYFDV |
| | 11 | RPWYFDV |
| | 19 | HATDY |
| | 27 | LGPHWYFDV |
| | 35 | HSGDAMDY |
| | 43 | WGIRQRGDY |
| | 51 | RGYFDY |
| | 59 | RGYFDY |
| | 67 | GGVRRYFDV |
| | 75 | GGVRRYFDV |
| | 99 | WGLRQGFAY |
| | 107 | SDGYPFGYSMAY |
| | 115 | RWLPRGLDF |
| | 123 | GGVRRYFDV |
| | 131 | GGYSPFAY |
| | 139 | DYGNYEDYAMDY |
| | 147 | GGVRRYFDV |
| | 155 | GGVRRYFDV |
| | 171 | GGVRRYFDV |
| | 179 | GGVRRYFDV |
| | 187 | WLLRYAMDY |
| LCDR1 | 4 | KASQNVGTAVA |
| | 12 | RSSQSIVHSNGNTYLQ |
| | 20 | SASSSVSYMH |
| | 28 | RSSQSLVYSNGNTYLH |
| | 36 | RASQSIGTNIH |
| | 44 | SASSSVSYLH |
| | 52 | KASQDINSYLS |
| | 60 | KASQDINKYIA |
| | 68 | RASQDIGSNLN |
| | 76 | RASQDIGSNLN |
| | 100 | KASQNVGGNVA |
| | 108 | HASQNINVWLS |
| | 116 | DIVMTQSHKFMSTSVGDRVSITC |
| | 124 | RASQDIGDNLN |
| | 132 | RSSQSLVHSNGNTYLH |
| | 140 | KASQSVDYDGDSYMN |
| | 148 | RASQDIGSNLI |
| | 156 | RASQDIGSNLN |
| | 172 | RSSQDIGSNLN |
| | 180 | RASQDIGSNLN |
| | 188 | KASQNVGNAVA |
| LCDR2 | 5 | SASNRYT |
| | 13 | KVSNRFS |
| | 21 | DTSKLAS |
| | 29 | KVSNRFS |
| | 37 | YASESIS |
| | 45 | DTSKLAS |
| | 53 | RANRLVD |
| | 61 | YTSTLQP |
| | 69 | ATSSLDS |
| | 77 | ATFSLDS |
| | 101 | STSYRNS |
| | 109 | KASNLHT |
| | 117 | WASTRHT |
| | 125 | ATFSLDS |
| | 133 | KVSNRFS |
| | 141 | AASNLES |
| | 149 | ATFNLDS |
| | 157 | ATFTLDS |
| | 173 | ATFSLDS |
| | 181 | ATFGLDS |
| | 189 | SASNRKN |
| LCDR3 | 6 | QQYSSYPLT |
| | 14 | FQGSHVPFT |
| | 22 | QQWSSNPLT |
| | 30 | SQSTPVPT |
| | 38 | QQSKSWPT |
| | 46 | QQWTSNPPT |
| | 54 | LQYDELYT |
| | 62 | LQYDNLYT |
| | 70 | LQYASFPYT |
| | 78 | LQYASFPYT |
| | 102 | QQYNSYPFT |
| | 110 | QQGQSYPFT |
| | 118 | QQYSTYYT |
| | 126 | LHYASFPYT |
| | 134 | SQNTHVPFT |
| | 142 | QQSNEDPPT |
| | 150 | LQYASSPYT |
| | 158 | LQYASFPYT |
| | 174 | LQYGSFPYT |
| | 182 | QQYASFPYT |
| | 190 | QQYSDSSWT |

TABLE 3
Amino acid sequences of each VH and VL of 21 exemplary
monoclonal antibodies.

| Antibody | VH (SEQ ID NO) | VL (SEQ ID NO) |
|---|---|---|
| 36A3G8 | QVQLQQSGAELARPGASVKMSC | DIVMTQSQKFMSTAVGDRVSI |
| | KASGYTFTTYTMHWIKQRPGQD | TCKASQNVGTAVAWYQQKPG |
| | LEWIGFINPSSGYTKYNQNFKDK | QSPKLLIYSASNRYTGVPDRF |
| | ATLTADKSSTTAYMQLNSLTSED | TGSGSGTDFTLTISNMQSEDL |
| | SAVYYCASPYGNIYFDVWGTGT | ADYFCQQYSSYPLTFGAGTKL |
| | TVTVSS (SEQ ID NO: 7) | ELK (SEQ ID NO: 8) |
| 28H9C12 | EVQLVESGGGLVKPGGSLKLSCA | DVLMTQTPLSLPVSLGDQASI |
| | ASGFTFSDYGMHWVRQAPEKG | SCRSSQSIVHSNGNTYLQWYL |
| | LEWVAYISSGSSTIYYADTVKGR | QKPGQSPKLLIYKVSNRFSGV |
| | FTISRDNAKNTLFLQMTSLRSED | PDRFSGSGSGTDFTLKISRLEA |
| | TAIYYCVRRPWYFDVWGTGTTV | EDLGVYYCFQGSHVPFTFGSG |
| | TVSS (SEQ ID NO: 15) | TKLEIK (SEQ ID NO: 16) |
| 26E2H6 | EVQLVESGGDLVKPGGSLKLSCA | QIVLTQSPAIMSASPGEKVTM |
| | ASGFTFSSYGMSWVRQTPDKRL | TCSASSSVSYMHWYQQKSGT |
| | EWVATISSGGSYTYYPDSVKGRF | SPKRWIYDTSKLASGVPARFS |
| | TISRDNAKNTLYLQMSSLKSEDT | GSGSGTSYSLTISSMEAEDAAT |
| | AMYYCARHATDYWGQGTTLTV | YYCQQWSSNPLTFGAGTKLE |
| | SS (SEQ ID NO: 23) | LK (SEQ ID NO: 24) |
| 20C4E1 | EVRLVESGGALVRPGGSLKLSCA | DVVLTQTPLSLPVSLGDQASIS |
| | ASGFTFSDYGMHWVRQAPEKG | CRSSQSLVYSNGNTYLHWFL |
| | LEWIAFISSVSFTIYYADTVKGRF | QKPGQSPQLLIYKVSNRFSGV |
| | TISRDNAKNTLFLQMTSLRSEDT | PDRFSGSGSGTDFTLKISRVEA |
| | ALYYCTRLGPHWYFDVWGTGT | EDLGVYFCSQSTPVPTFGGGT |
| | TVTVSS (SEQ ID NO: 31) | KLEIK (SEQ ID NO: 32) |
| 15D6G2 | EVQLVESGGDLVKPGGYLRLSC | DILLTQSPAILSVSPGERVSFSC |
| | AASGFTFSSHGMSWVRQTPDKR | RASQSIGTNIHWYQQRTNGSP |
| | LEWVATISSGGRNTYYPDSVKGR | RLLIKYASESISGIPSRFSGSGS |
| | FTISRDNAKNILYLQMSSLKSED | GTDFTLSINSVESEDIADYYCQ |
| | TAMFYCTRHSGDAMDYWGQGT | QSKSWPTFGGGTKLEIK (SEQ |
| | SVTVSS (SEQ ID NO: 39) | ID NO: 40) |
| 12G12C2 | QIQLVQSGPELKMPGETVKISCK | QIVLTQSPAIMSASPGEKVTM |
| | ASGYTFTTYGLSWVKQAPGKGL | TCSASSSVSYLHWYQQKSGTS |
| | KWMGWINTFSGVPIYADDFKGR | PKRWIYDTSKLASGVPVRFSG |
| | FAFSLETSASTAYLQINNLKNEDT | SGSETSYSLTISSMEAEDAATY |
| | ATYFCARWGIRQRGDYWGQGA | YCQQWTSNPPTFGGGTKLEIK |
| | TLTVSS (SEQ ID NO: 47) | (SEQ ID NO: 48) |
| 71F8H2 | EVQLVESGGGLVKPGGSLKLSCA | DIKMTQSPSSMYASLGERVTIT |
| | ASGFTFSSYAMSWVRQTPEKRL | CKASQDINSYLSWFQQKPGKS |
| | EWVATISDGGSYTYYPDNVKGR | PKTLIYRANRLVDGVPSRFSG |
| | FTISRDNAKNNLYLQMSHLKSED | SGSGQDYSLTISSLEYEDMGIY |
| | TAMYYCARRGYFDYWGQGTTL | YCLQYDELYTFGGGTKLEIK |
| | TVSS (SEQ ID NO: 55) | (SEQ ID NO: 56) |
| 64G9C1 | EVQLVESGGGLVKPGGSLKLSCA | DIQMTQSPSSLSASLGGKVTIT |
| | ASGFTFSSYAMSWVRQTPEKRL | CKASQDINKYIAWYQHKPGK |
| | EWVATISDGGSYTYYPDNVKGR | GPRLLIHYTSTLQPGIPSRFSGS |
| | FTISRDNAKNNLYLQMSHLKSED | GSGRDYSFSISNLEPEDIATYY |
| | TAMYYCTRRGYFDYWGQGTTL | CLQYDNLYTFGGGTKLEIK |
| | TVSS (SEQ ID NO: 63) | (SEQ ID NO: 64) |
| 98A3A8 | EVQLQQSGPELVKPGASVKMSC | DIQMTQSPSSLSASLGERVSLT |
| | KASGYTFTYYVTHWVRQKPGQ | CRASQDIGSNLNWLQQKPDG |
| | GLEWIGYINPYNDGTMFNEKFK | TIKRLIYATSSLDSGVPKRFSG |
| | GKATLTSDKSSSTAYMEFSSLTSE | SRSGSDYSLTINSLESEDFVDY |
| | DSAVFYCARGGVRRYFDVWGA | YCLQYASFPYTFGGGTKLEIK |
| | GTTVTVSS (SEQ ID NO: 71) | (SEQ ID NO: 72) |
| 96C9E7 | EVQLQQSGPELVKPGASVKMSC | DIQMTQSPSSLSASLGERVSLT |
| | KASGYTFTYYILHWVKQKPGQG | CRASQDIGSNLNWLQQEPDG |
| | LEWIGYFNPYNDGNKFNERFRG | TIKRLISATFSLDSGVPKRFSGS |
| | KATLTSDKSSSTAYMELNSLTSED | RSGSDYSLTISSLESEDFVNYY |
| | SAVYFCARGGVRRYFDVWGAG | CLQYASFPYTFGGGTKLEIK |
| | TTVTVSS (SEQ ID NO: 79) | (SEQ ID NO: 80) |
| 90G6H3 | QAYLQQSGAELVRSGASVKMSC | DIVMTQSQKFMSTSVGDRVS |
| | KASGYTFTSYIMHWVKQTPGQG | VTCKASQNVGGNVAWYQQK |
| | LEWIGYIYPGNGGTNYNQKFKD | PGQSPKALIYSTSYRNSGVPD |
| | KATLSADTSSSTAYMQINSLTSED | RFTGSGSGTDFTLTISNVQSED |
| | SAVYFCARWGLRQGFAYWGQG | LAEYFCQQYNSYPFTFGSGTK |
| | TLVSVSA (SEQ ID NO: 103) | LEIR (SEQ ID NO: 104) |
| 89F11E3 | QVQLQQSGAELARPGASVKMSC | DIQMNQSPSSLSASLGDTITIT |
| | KASGYTFTSSTIHWVKQRPGQG | CHASQNINVWLSWYHQKPGN |
| | LAWIGYIVPSTGYTNYNQKFKD | IPKLLIYKASNLHTGVPSRFSG |
| | KATLTADKSSSTAYMQLTSLTSED | SGSGTGFTLTISRLQPEDIATY |
| | SAVYYCARSDGYPFGYSMAYW | YCQQGQSYPFTFGSGTKLEIK |
| | GQGTSVTVSS (SEQ ID NO: 111) | (SEQ ID NO: 112) |
| 87A3G8 | QVQLKESGPGLVAPSQSLSITCTV | DIVMTQSHKFMSTSVGDRVSI |
| | SGFSLSSYSVYWVRQPPGKGLE | TCKASQDVGTAVAWYQQKPG |
| | WLGVIWAGGSTNYNSALMSRLS | QSPKLLIYWASTRHTGVPDRF |
| | INKDKSKSQVFLKMNSLQTDDS | TGSGSGTDFTLTISNVQSEDLA |
| | AIYYCAMRWLPRGLDFWGQGT | DYFCQQYSTYYTFGGGTKLEI |
| | SVTVSS (SEQ ID NO: 119) | K (SEQ ID NO: 120) |
| 83A9B8 | EVQLQQSGPELVKPGASVKMSC | DIQMTQSPSSLSASLGERVSLT |
| | KASGYTFSNYVIHWVKQKPGQG | CRASQDIGDNLNWLQQEPDG |
| | LEWIGYFNPYNDGSKYNEKFKG | TIKRLIYATFSLDSGVPKRFSG |
| | KATLTSDKSSSTAYMELSSLTSED | SRSGSVYSLTISRLESEDFVDY |
| | SAVYYCARGGVRRYFDVWGAG | FCLHYASFPYTFGGGTKLEIK |
| | TTVTVSS (SEQ ID NO: 127) | (SEQ ID NO: 128) |
| 82A1C10 | QVQLKESGPGLVAPSQSLSITCTV | DVVMTQTPLSLPVSLGDQASI |
| | SGFSLTNYAVTWVRQPPGKGLE | SCRSSQSLVHSNGNTYLHWY |
| | WLGVIWGDGSTYYHSALVSRLSI | LQKPGQSPKLLIFKVSNRFSG |
| | TKDNSRTQVLLKLNSLQTDDTA | VPDRFSGSGSGTDFTLKISRVE |
| | RYYCAKGGYSPFAYWGQGTLVT | AEDLGVYFCSQNTHVPFTFGS |
| | VSA (SEQ ID NO: 135) | GTKLEIK (SEQ ID NO: 136) |
| 119E4B4 | EVQLQQSGAELVKPGASVKLSC | DIVLTQSPASLAVSLGQRATIS |
| | TASGFNIKDTYMHWVKQRPEQG | CKASQSVDYDGDSYMNWYQ |
| | LEWIGRIDPANGNTKYDPKFQG | QKPGQPPKLLIYAASNLESGIP |
| | KATITADTSSNTAYLQLSSLTSED | ARFSGSGSGTDFTLNIHPVEEE |
| | TAVYYCARDYGNYEDYAMDYW | DAATYYCQQSNEDPPTFGGG |
| | GQGTSVTVSS (SEQ ID NO: 143) | TKLEIK (SEQ ID NO: 144) |
| 116G1G4 | EVQLQQSGPELVKPGASVKMSC | DIQMTQSPSSLSASLGERVILT |
| | KASGYTFTYYIMHWVKQEPGQ | CRASQDIGSNLIWLQQKPDGT |
| | GLQWIGYINPYNDGAYYSARFK | IKRLIYATFNLDSGVSKRFSGS |
| | GKATLTSDKSSSTAYMDLTSLTSE | RSGSDYSLTISSLDSEDFVDYY |
| | DSAVYYCARGGVRRYFDVWGA | CLQYASSPYTFGGGTKLEIK |
| | GTTVTVSS (SEQ ID NO: 151) | (SEQ ID NO: 152) |
| 116F1G6 | EVQLQHSGPELVKPGASVKMSC | DIQMTQSPSSLSASLGERVSLT |
| | KASGYTFTTYVMHWVKQKPGQ | CRASQDIGSNLNWLQQEPNG |
| | GLEWIGYINPYNDGTKYNEKFK | TIKRLIYATFTLDSGVPKRFSG |
| | GKATLTSDKSSSTAYMELSSLTSE | SRSGSDYSLTISSLESEDFVDY |
| | DSAVYYCARGGVRRYFDVWGA | YCLQYASFPYTFGGGTKLEIK |
| | GTTVTVSS (SEQ ID NO: 159) | (SEQ ID NO: 160) |
| 110B4H5 | EVQLQQSGPELVKPGASVKMSC | DIQMTQSPSSLSASLGERVSLT |
| | KASGYTFTFYIMHWVKQKPGQG | CRSSQDIGSNLNWLQQEPDGT |
| | PEWIGYINPYNDGTKYNAKFKG | IKRLIYATFSLDSGVPKRFSGS |
| | KATLSSDRSSSTAYMELSSLTSED | RSGSHYSLTISSLESEDFVVYY |
| | SAVYYCARGGVRRYFDVWGAG | CLQYGSFPYTFGGGTKLEIK |
| | TPVTVSS (SEQ ID NO: 175) | (SEQ ID NO: 176) |
| 108C4C12 | EVQLQQSGPELVKPGASVKMSC | DIQMTQSPSSLSASLGERVSLT |
| | KASGYTFTSYVIHWVKQKTGQG | CRASQDIGSNLNWLQQEPDG |
| | LEWIGYFNPYNDGTKYNEEFKG | TIKRLIYATFGLDSGVPKRFSG |
| | KATLTSDKSSNTAYMELNSLTSD | SRSGSDYSLTISSLESEDFVDY |
| | DSAVYFCARGGVRRYFDVWGA | YCQQYASFPYTFGGGTKLEIK |
| | GTTVTVSS (SEQ ID NO: 183) | (SEQ ID NO: 184) |
| 101B3G3 | EVQLQQSGPELVKPGASMKISCK | DIVMTQSQKFMSTSVGDRVSI |
| | ASGYSFTGYTMNWVKQSHGKN | TCKASQNVGNAVAWYQQKPG |
| | LEWIGLINPYNGGTRYNQKFKD | HSPKLLISSASNRKNGVPDRF |
| | KATLTVDKSSSTAYMELLSLTSE | TGSGSGTDFTLTVSNMQSEDL |
| | DSAVYYCARWLLRYAMDYWGQ | ADYFCQQYSDSSWTFGGGTK |
| | GTSVTVSS (SEQ ID NO: 191) | LEIK (SEQ ID NO: 192) |

Given that each of the 21 exemplary monoclonal antibodies can bind to GPRC5D and that antigen-binding specificity is provided primarily by the CDR1, CDR2 and CDR3 regions, the HCDR1, HCDR2 and HCDR3 sequences and LCDR1, LCDR2 and LCDR3 sequences of each of the 21 exemplary monoclonal antibodies can be “mixed and matched” (i.e., CDRs from different antibodies can be mixed and matched, but each antibody must contain a HCDR1, HCDR2 and HCDR3 and a LCDR1, LCDR2 and LCDR3) to create anti-GPRC5D antibodies or antigen-binding fragments thereof of the present disclosure. GPRC5D binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence(s). Likewise, when VL CDR sequences are mixed and matched, the LCDR1, LCDR2 and/or LCDR3 sequence from a particular VL sequence preferably is replaced with a structurally similar CDR sequence(s). For example, the HCDR1s of antibodies 26E2H6 and 71F8112 share some structural similarity and therefore are amenable to mixing and matching. It will be readily apparent to a person skilled in the art that novel VH and VL sequences can be created by substituting one or more VH and/or VL CDR sequences with structurally similar sequences from the CDR sequences disclosed herein for the 21 exemplary monoclonal antibodies.

CDRs are known to be responsible for antigen binding. However, it has been found that not all of the 6 CDRs are indispensable or unchangeable. In other words, it is possible to replace or change or modify one or more CDRs in each of the 21 exemplary monoclonal antibodies, yet substantially retain the specific binding affinity to GPRC5D.

In certain embodiments, the anti-GPRC5D antibodies and the antigen-binding fragments provided herein comprise a heavy chain CDR3 sequence of one of the anti-GPRC5D antibodies 36A3G8, 28H9C12, 26E2H6, 20C4E1, 15D6G2, 12G12C2, 71F8H2, 64G9C1, 98A3A8, 96C9E7, 90G6H3, 89F11E3, 87A3G8, 83A9B8, 82A1C10, 119E4B4, 116G1G4, 116F1G6, 110B4H5, 108C4C12 or 101B3G3. In certain embodiments, the anti-GPRC5D antibodies and the antigen-binding fragments thereof provided herein comprise a heavy chain CDR3 sequence selected from the group consisting of SEQ ID NOs: 3, 11, 19, 27, 35, 43, 51, 59, 67, 75, 99, 107, 115, 123, 131, 139, 147, 155, 171, 179, and 187. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity, 13:37-45) as well as desirable antigen-binding affinity (Schier R, et al, J Mol Biol. 263:551-67).

In certain embodiments, the antibodies or antigen-binding fragments thereof provided herein comprise a VH region having an amino acid sequence as set forth in SEQ ID NOs: 7, 15, 23, 31, 39, 47, 55, 63, 71, 79, 103, 111, 119, 127, 135, 143, 151, 159, 175, 183, or 191, or a homologous sequence thereof having at least 80% (e.g., at least 85%, at least 90%, at least 91%, at least 92%, at least 93%, at least 94%, at least 95%, at least 96%, at least 97%, at least 98%, at least 99%) sequence identity to SEQ ID NOs: 7, 15, 23, 31, 39, 47, 55, 63, 71, 79, 103, 111, 119, 127, 135, 143, 151, 159, 175, 183, or 191.

In certain embodiments, the antibodies or antigen-binding fragments thereof provided herein comprise a VL region having an amino acid sequence as set forth in SEQ ID NOs: 8, 16, 24, 32, 40, 48, 56, 64, 72, 80, 104, 112, 120, 128, 136, 144, 152, 160, 176, 184, or 192, or a homologous sequence thereof having at least 80% (e.g., at least 85%, at least 90%, at least 91%, at least 92%, at least 93%, at least 94%, at least 95%, at least 96%, at least 97%, at least 98%, at least 99%) sequence identity to SEQ ID NOs: 8, 16, 24, 32, 40, 48, 56, 64, 72, 80, 104, 112, 120, 128, 136, 144, 152, 160, 176, 184, or 192.

In certain embodiments, the antibodies or antigen-binding fragments thereof provided herein comprise a VH/VL amino acid sequence pair selected from the group consisting of SEQ ID NOs: 7/8, 15/16, 23/24, 31/32, 39/40, 47/48, 55/56, 63/64, 71/72, 79/80, 103/104, 111/112, 119/120, 127/128, 135/136, 143/144, 151/152, 159/160, 175/176, 183/184, or 191/192.

In certain embodiments, the antibodies and antigen-binding fragments thereof provided herein comprise suitable framework region (FR) sequences, as long as the antibodies and antigen-binding fragments thereof can bind to GPRC5D. The CDR sequences provided in Table 2 above are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment thereof is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al., (1986) Nature 321:522-525; Riechmann et at, (1988) Nature 332:323-327; Verhocyen et al, (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al, (1993) J. Immunol 151:2296; Chothia et al., (1987) J. Mot. Biol 196:901). Alternatively, a framework derived from the consensus sequence of all human antibodies may be used for the grafting of the non-human CDRs (see, for example, Carter et al, (1992) Proc. Natl. Acad. Sci. USA, 89:4285; Presta et al, (1993) J. Immunol, 151:2623).

In some embodiments, the anti-GPRC5D antibodies or antigen-binding fragments thereof provided herein are humanized. In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1, HFR2, HFR3 and HFR4, and/or light chain LFR1, LFR2, LFR3 and LFR4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In some embodiments, the anti-GPRC5D antibodies and antigen-binding fragments thereof provided herein comprise all or a portion of the heavy chain variable domain and/or all or a portion of the light chain variable domain. In one embodiment, the anti-GPRC5D antibody or antigen-binding fragment thereof provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-GPRC5D antibodies or antigen-binding fragments thereof provided herein further comprise an Fc region. In certain embodiments, the anti-GPRC5D antibodies or antigen-binding fragments thereof provided herein further comprise an Fc region of human immunoglobulin (Ig). In certain embodiments, the anti-GPRC5D antibodies or antigen-binding fragments thereof provided herein further comprise a constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-GPRC5D antibodies or antigen-binding fragments thereof provided herein comprise heavy chain constant regions of human IgG1, IgG2, IgG3, IgG4, IgA1, IgA2 or IgM. In certain embodiments, the anti-GPRC5D antibodies or antigen-binding fragments thereof provided herein comprise a lambda (λ) light chain or a kappa (κ) light chain. The constant region of the anti-GPRC5D antibodies or antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-GPRC5D antibodies and antigen-binding fragments thereof provided herein comprises an Fc region of IgG1, or IgG3 isotype, which could induce ADCC or CDC; or alternatively, a constant region of IgG4 or IgG2 isotype, which has reduced or depleted effector function. In some embodiments, the Fe region derived from human IgG1 with enhanced effector functions. In some embodiments, the Fc region comprises an amino acid sequence as set forth in SEQ ID NO: 161.

(SEQ ID NO: 161)
ASTKGPSVFPLAPSSKSTSGGTAALGCLVKDYFPEPVTVSWNSG
ALTSGVHTFPAVLQSSGLYSLSSVVTVPSSSLGTQTYICNVNHK
PSNTKVDKKVEPKSCDKTHTCPPCPAPELLGGPSVFLFPPKPKD
TLMISRTPEVTCVVVDVSHEDPEVKFNWYVDGVEVHNAKTKPRE
EQYNSTYRVVSVLTVLHQDWLNGKEYKCKVSNKALPAPIEKTIS
KAKGQPREPQVYTLPPSREEMTKNQVSLTCLVKGFYPSDIAVEW
ESNGQPENNYKTTPPVLDSDGSFFLYSKLTVDKSRWQQGNVFSC
SVMHEALHNHYTQKSLSLSPGK

In certain embodiments, the antibodies or antigen-binding fragments thereof provided herein have a specific binding affinity to human GPRC5D which is sufficient to provide for diagnostic and/or therapeutic use.

The antibodies or antigen-binding fragments thereof provided herein can be a monoclonal antibody, a polyclonal antibody, a humanized antibody, a human antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-GPRC5D antibody or antigen-binding fragment thereof, which competes for binding to GPRC5D with the antibody or antigen-binding fragment thereof provided herein. In certain embodiments, the present disclosure provides an anti-GPRC5D antibody or antigen-binding fragment thereof, which competes for binding to human GPRC5D with any one of antibodies 36A3G8, 28H9C12, 26E2H6, 20C4E1, 15D6G2, 12G12C2, 71F8H2, 64G9C1, 98A3A8, 96C9E7, 90G6H3, 89F11E3, 87A3G8, 83A9B8, 82A1C10, 119E4B4, 116G1G4, 116F1G6, 110B4H5, 108C4C12 or 101B3G3. In some embodiments, the present disclosure provides an anti-GPRC5D antibody or antigen-binding fragment thereof, which competes for the same epitope with the antibody or antigen-binding fragment thereof provided herein.

The ability to “block binding” or “compete for the same epitope” as used herein refers to the ability of an antibody or antigen-binding fragment to inhibit the binding interaction between two molecules (e.g., human GPRC5D and an anti-GPRC5D antibody) to any detectable degree. In certain embodiments, an antibody or antigen-binding fragment that blocks binding between two molecules inhibits the binding interaction between the two molecules by at least 85%, or at least 90%. In certain embodiments, this inhibition may be greater than 85%, or greater than 90%.

Those skilled in the art will recognize that it is possible to determine, without undue experimentation, if a human monoclonal antibody binds to the same epitope as the antibody of present disclosure (e.g., mouse monoclonal antibodies 36A3G8, 281H9C12, 26E2H6, 20C4E1, 15D6G2, 12G12C2, 71F8H2, 64G9C1, 98A3A8, 96C9E7, 90G6H3, 89F11E3, 87A3G8, 83A9B8, 82A1C10, 119E4B4, 116G1G4, 116F1G6, 110B4H5, 108C4C12 or 101B3G3) by ascertaining whether the former prevents the latter from binding to a GPRC5D antigen polypeptide. If the test antibody competes with the antibody of the present disclosure, as shown by a decrease in binding by the antibody of present disclosure to the GPRC5D antigen polypeptide, then the two antibodies bind to the same, or a closely related, epitope. Or if the binding of a test antibody to the GPRC5D antigen polypeptide was inhibited by the antibody of the present disclosure, then the two antibodies bind to the same, or a closely related, epitope.

In certain embodiments, the present disclosure provides anti-GPRC5D antibodies or antigen-binding fragments thereof which compete for binding to GPRC5D with ATG596. In certain embodiments, the anti-GPRC5D antibody or antigen-binding fragment which competes for binding to GPRC5D with the antibody or antigen-binding fragment thereof provided herein is not ATG596.

“ATG596” as used herein refers to an antibody or antigen-binding fragment thereof comprising one or two or three heavy chain CDRs contained within a heavy chain variable region having an amino acid sequence of SEQ ID NO: 199, and one or two or three light chain CDRs contained within a light chain variable region having an amino acid sequence of SEQ ID NO: 200. In certain embodiments, ATG596 comprises a HCDR1 comprising an amino acid sequence of SEQ ID NO: 193, a HCDR2 comprising an amino acid sequence of SEQ ID NO: 194, a HCDR3 comprising an amino acid sequence of SEQ ID NO: 195, a LCDR1 comprising an amino acid sequence of SEQ ID NO: 196, a LCDR2 comprising an amino acid sequence of SEQ ID NO: 197, and a LCDR3 comprising an amino acid sequence of SEQ ID NO: 198. The amino acid sequences of each CDR, VII region and VL region of ATG596 are shown in Table 4 below. The CDR boundaries of ATG596 in Table 4 below are identified by the convention of Kabat.

TABLE 4
Amino acid sequences of HCDRs, LCDRs,
VH region and VL region of ATG596

| Sequence | SEQ ID | |
|---|---|---|
| Description | NO | Amino Acid Sequence |
| HCDR1 | 193 | GYTMN |
| HCDR2 | 194 | LINPYNSDTNYAQKLQG |
| HCDR3 | 195 | VALRVALDY |
| LCDR1 | 196 | KASQNVATHVG |
| LCDR2 | 197 | SASYRYS |
| LCDR3 | 198 | QQYNRYPYT |
| VH | 199 | QVQLVQSGAEVKKPGASVKVS |
| | | CKASGYSFTGYTMNWVRQAPG |
| | | QGLEWMGLINPYNSDTNYAQK |
| | | LQGRVTMTTDTSTSTAYMELR |
| | | SLRSDDTAVYYCARVALRVAL |
| | | DY |
| | | WGQGTLVTVSS |
| VL | 200 | DIQMTQSPSSLSASVGDRVTI |
| | | TCKASQNVATHVGWYQQKPGK |
| | | APKRLIYSASYRYSGVPSRFS |
| | | GSGSGTEFTLTISNLQPEDFA |
| | | TYYCQQYNRYPYTFGQGTKLE |
| | | IK |

### Antibody Variants

The antibodies and antigen-binding fragments thereof provided herein also encompass various variants of the antibody sequences provided herein.

In certain embodiments, the antibody variants comprise one or more amino acid residue substitutions or modifications yet retains binding affinity to GPRC5D. In certain embodiments, at least one of the substitutions or modifications is in one or more of the CDR sequences of the VH region or VL region. In certain embodiments, at least one of the substitutions or modifications is in one or more of the non-CDR sequences of the VH region or VL region. In certain embodiments, the antibodies or antigen-binding fragments thereof provided herein further comprise one or more non-natural amino acid (NNAA) substitution. In certain embodiments, the NNAA is capable of being conjugated.

For example, the antibody variants comprise one or more amino acid residue substitutions or modifications in one or more of the CDR sequences provided in Table 2 above, one or more of the non-CDR sequences of the heavy chain variable region or light chain variable region provided in Table 3 above, and/or the constant region (e.g., Fc region), Such variants retain binding specificity to GPRC5D of their parent antibodies, but have one or more desirable properties conferred by the modification(s) or substitution(s). For example, the antibody variants may have improved antigen-binding affinity, improved glycosylation pattern, reduced risk of glycosylation, reduced deamination, enhanced effector function(s), improved FcRn receptor binding, increased pharmacokinetic half-life, pH sensitivity, and/or compatibility to conjugation (e.g., one or more introduced cysteine residues), etc.

The parent antibody sequence may be screened to identify suitable or preferred residues to be modified or substituted, using methods known in the art, for example, “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### Affinity Variants

Affinity variants of antibodies may contain modifications or substitutions in one or more CDR sequences provided in Table 2 above, one or more FR sequences, or the heavy or light chain variable region sequences provided in Table 3 above. FR sequences can be readily identified by a person skilled in the art based on the CDR sequences in Table 2 above and variable region sequences in Table 3 above, as it is well-known in the art that a CDR region is flanked by two FR regions in the variable region. The affinity variants retain specific binding affinity to GPRC5D of the parent antibody, or even have improved GPRC5D binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Table 2 above, and variable region sequences provided in Table 3 above, one or more amino acid residues may be substituted yet the resulting antibody or antigen-binding fragment still retain the binding affinity or binding capacity to GPRC5D, or even have an improved binding affinity or capacity. Various methods known in the art can be used to achieve this purpose. For example, a library of antibody variants (such as Fab or scFv variants) can be generated and expressed with phage display technology, and then screened for the binding affinity to human GPRC5D. For another example, computer software can be used to virtually simulate the binding of the antibodies to human GPRC5D, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the anti-GPRC5D antibodies or antigen-binding fragments thereof provided herein comprise 1, 2, or 3 CDR sequences having at least 80% (e.g., at least 85%, 88%, 90%, 91%, 92%, 93%, 94%, 95%, 96%, 97%, 98%, or 99%) sequence identity to that (or those) listed in Table 2 above yet retaining the specific binding affinity to GPRC5D at a level similar to or even higher than its parent antibody.

In certain embodiments, the anti-GPRC5D antibodies or antigen-binding fragments thereof comprise one or more variable region sequences having at least 80% (e.g., at least 85%, 88%, 90%, 91%, 92%, 93%, 94%, 95%, 96%, 97%, 98%, or 99%) sequence identity to that (or those) listed in Table 3 above yet retaining the specific binding affinity to GPRC5D at a level similar to or even higher than its parent antibody. In some embodiments, a total of 1 to 10 amino acids have been substituted, inserted, or deleted in a variable region sequence listed in Table 3 above. In some embodiments, the substitutions, insertions, or deletions occur in regions outside the CDRs (e.g., in the FRs).

### Glycosylation Variants

The anti-GPRC5D antibodies or antigen-binding fragments thereof provided herein also encompass glycosylation variants, which can be obtained to either increase or decrease the extent of glycosylation of the antibodies or antigen-binding fragments thereof.

The antibodies or antigen-binding fragments thereof provided herein may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyanino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

### Cysteine-Engineered Variants

The anti-GPRC5D antibodies or antigen-binding fragments thereof provided herein also encompass cysteine-engineered variants, which comprise one or more introduced free cysteine amino acid residues.

A free cysteine residue is one which is not part of a disulfide bridge, A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-GPRC5D antibodies and antigen-binding fragments provided herein also encompass an Fc variant, which comprises one or more amino acid residue modifications or substitutions at its Fc region and/or hinge region.

In certain embodiments, the anti-GPRC5D antibodies or antigen-binding fragments thereof provided herein comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70: 3269-3277 (2010); and Hinton, P. et al., J. Immunology, 176:346-356 (2006).

In certain embodiments, the anti-GPRC5D antibodies or antigen-binding fragments thereof provided herein comprise one or more amino acid substitution(s) that alters ADCC. Certain amino acid residues at CH2 domain of the Fc region can be substituted to provide for enhanced ADCC activity. Alternatively or additionally, carbohydrate structures on the antibody can be changed to enhance ADCC activity. Methods of altering ADCC activity by antibody engineering have been described in the art, see for example, Shields R L. et al., J Biol Chem. 2001. 276(9): 6591-604; Idusogie E E. et al., J Immunol. 2000. 164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O, et al., Mol Cancer Ther. 2008, 7(8): 2517-27; Shields R. L. et al., J. Biol. Chem, 2002, 277: 26733-26740; Shinkawa T. et al., J. Biol. Chem, 2003, 278: 3466-3473.

In certain embodiments, the anti-GPRC5D antibodies or antigen-binding fragments thereof comprise one or more amino acid substitution(s) that alters CDC, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants. One or more amino acids selected from amino acid residues 329, 331 and 322 of the Fc region can be replaced with a different amino acid residue to alter C1q binding and/or enhance CDC (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-GPRC5D antibodies or antigen-binding fragments thereof provided herein comprise one or more amino acid substitution(s) in human immunoglobulin (e.g., IgG1) at position 234 and/or 235 (according to EU numbering). In certain embodiments, the anti-GPRC5D antibodies or antigen-binding fragments thereof provided herein comprise two amino acid substitutions in human immunoglobulin (e.g., IgG1) at positions 234 and 235 (according to EU numbering). In certain embodiments, the anti-GPRC5D antibodies or antigen-binding fragments thereof provided herein comprise L234A and L235A (according to EU numbering) amino acid substitutions.

In certain embodiments, the anti-GPRC5D antibodies or antigen-binding fragments thereof provided herein comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

In certain embodiments, the anti-GPRC5D antibodies or antigen-binding fragments thereof provided herein comprise an amino acid substitution at position 366 (according to EU numbering) of a first Fc polypeptide, and comprises one, two or three amino acid substitutions at one, two or three positions of 366, 368, and 407 (according to EU numbering) of a second Fc polypeptide. In certain embodiments, the anti-GPRC5D antibodies or antigen-binding fragments thereof provided herein comprise a T366W substitution (according to EU numbering) of a first Fc polypeptide, and comprise T366S+L368A+Y407V substitutions (according to EU numbering) of a second Fc polypeptide.

In certain embodiments, the anti-GPRC5D antibodies or antigen-binding fragments thereof provided herein further comprise one or more amino acid substitution(s) of a first Fc polypeptide, and comprise one or more amino acid substitution(s) of a second Fc polypeptide, so as to introduce a non-natural disulfide bond between the two Fc polypeptides. For example, the anti-GPRC5D antibodies or antigen-binding fragments thereof provided herein comprise an amino acid substitution at position 354 (according to EU numbering) of a first Fc polypeptide, and comprise an amino acid substitution at position 349 (according to EU numbering) of a second Fc polypeptide. In certain embodiments, the anti-GPRC5D antibodies or antigen-binding fragments thereof provided herein comprise S354C substitution (according to EU numbering) of a first Fc polypeptide, and comprise Y349C substitution (according to EU numbering) of a second Fc polypeptide.

In certain embodiments, the anti-GPRC5D antibodies or antigen-binding fragments thereof provided herein comprises one or more (e.g., 2, 3, 4, 5, 6, 7 or 8) amino acid substitutions selected from the group consisting of: L234A, L235A, S354C, T366W, Y349C, T366S, L368A, and Y407V (according to EU numbering). In certain embodiments, the anti-GPRC5D antibodies or antigen-binding fragments thereof provided herein comprise S354C and T366W (according to EU numbering) amino acid substitutions of a first Fc polypeptide, and comprise Y349C, T366S, L368A, and Y407V (according to EU numbering) amino acid substitutions of a second Fc polypeptide.

In certain embodiments, anti-GPRC5D antibodies or antigen-binding fragments thereof provided herein comprise a first Fc polypeptide comprising the amino acid sequence as set forth in SEQ ID NO: 162, and a second Fc polypeptide comprising the amino acid sequence as set forth in SEQ ID NO: 163.

(SEQ ID NO: 162)
KTHTCPPCPAPEAAGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPCRE
EMTKNQVSLWCLVKGFYPSDIAVEWESNGQPENNYKTTPPVLDSD
GSFFLYSKLTVDKSRWQQGNVFSCSVMHEALHNHYTQKSLSLSPG
K
(SEQ ID NO: 163)
DKTHTCPPCPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLSCAVKGFYPSDIAVEWESNGQPENNYKTTPPVLDS
DGSFFLVSKLTVDKSRWQQGNVFSCSVMHEALHNHYTQKSLSLSP
GK

### Antigen-Binding Fragments

Provided herein are also anti-GPRC5D antigen-binding fragments. Various types of antigen-binding fragments are known in the art and can be developed based on the anti-GPRC5D antibodies provided herein, including for example, the exemplary antibodies whose CDRs are shown in Table 2 above, and variable sequences are shown in Table 3 above, and their different variants (such as affinity variants, glycosylation variants, Fc variants, cysteine-engineered variants and so on).

In certain embodiments, an anti-GPRC5D antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g., for Fab, Fv and ScFv antibody fragments), screening from a phage display library as discussed above (e.g., for ScFv), and chemical coupling of two Fab′-SH fragments to form F(ab′)₂ fragments (Carter et al., Bio/Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-GPRC5D antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e., having the same sequences), or structurally different (i.e., having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific or Multi-Specific Antibodies

In certain embodiments, the anti-GPRC5D antibody or antigen-binding fragment thereof provided herein is bispecific or multi-specific. In certain embodiments, the anti-GPRC5D antibody or antigen-binding fragment thereof provided herein is further linked to a second binding moiety having a different binding specificity from said anti-GPRC5D antibody or antigen-binding fragment thereof. In some embodiments, the bispecific or multi-specific antibody or antigen-binding fragment thereof provided herein has a first specificity for GPRC5D, and a second specificity. In some embodiments, the second specificity is for GPRC5D but to different epitopes. In some embodiments, the second specificity is for a second antigen different from GPRC5D.

In certain embodiments, the second specificity is for a tumor associated antigen or an epitope thereof. The term “tumor associated antigen” refers to an antigen that is or can be presented on a tumor cell surface and that is located on or within tumor cells. In some embodiments, the tumor associated antigens can be presented only by tumor cells and not by normal cells, i.e., non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but are overexpressed on tumor cells when compared to non-tumor cells or are accessible for antibody binding in tumor cells due to the less compact structure of the tumor tissue compared to non-tumor tissue. In some embodiments, the tumor associated antigen is located on the vasculature of a tumor.

In certain embodiments, the bispecific or multi-specific antibodies or antigen-binding fragments thereof provided herein are capable of binding to one or more (e.g., 1, 2, 3, 4, 5 or more) additional antigens other than GPRC5D. In certain embodiments, the one or more additional antigens other than GPRC5D are selected from the group consisting of KRAS, ERK, XPO1, mTORC1/2, PAK4, NAMPT, ATR, EGFR, FGFR, VEGF, LILRB, c-MET, Her2, Her3, CTLA4, GITA, CD112R, CD2, CD3, CD7, CD16, CD19, CD20, CD24, CD27, CD30, CD34, CD37, CD38, CD39, CD70, CD73, CD83, CD28, CD80 (B7-1), CD86 (B7-2), CD40, CD40L (CD154), CD47, SIRPα, CD122, CD137, CD137L, OX40 (CD134), OX40L (CD252), BCMA (e.g., BCMA02), FcRH5, PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), BTLA, CD160, CTLA-4 (CD152), IDO1, IDO2, ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), and TGFβ.

In certain embodiments, the bispecific or multi-specific antibodies or antigen-binding fragments thereof provided herein bind to GPRC5D and CD3. In certain embodiments, the bispecific or multi-specific antibodies or antigen-binding fragments thereof provided herein bind to GPRC5D and BCMA. In certain embodiments, the bispecific or multi-specific antibodies or antigen-binding fragments thereof provided herein bind to GPRC5D and CD38. In certain embodiments, the bispecific or multi-specific antibodies or antigen-binding fragments thereof provided herein bind to GPRC5D and CD19. In certain embodiments, the bispecific or multi-specific antibodies or antigen-binding fragments thereof provided herein bind to GPRC5D and FcRH5. In certain embodiments, the bispecific or multi-specific antibodies or antigen-binding fragments thereof provided herein bind to GPRC5D and PD-L1. In certain embodiments, the bispecific or multi-specific antibodies or antigen-binding fragments thereof provided herein bind to GPRC5D and PD-1.

In certain embodiments, the bispecific or multi-specific antibody or antigen-binding fragment thereof provided herein comprises a GPRC5D binding moiety and a CD3 binding moiety.

As used herein, the term “GPRC5D binding moiety” with regard to a bispecific or multi-specific antibody or antigen-binding fragment thereof, refers to a moiety that is capable of binding to GPRC5D (e.g., human GPRC5D, mouse GPRC5D, and cynomolgus GPRC5D). The GPRC5D binding moiety can take any form that allows specific recognition of the target GPRC5D. For example, the GPRC5D binding moiety may be an antibody or an antigen-binding fragment thereof, e.g., an IgG (such as IgG1, IgG2, IgG3, and IgG4) antibody, IgA antibody, or IgM antibody.

The GPRC5D binding moiety of the bispecific or multi-specific antibodies provided herein may be derived from any of the anti-GPRC5D antibodies described above. In certain embodiments, the GPRC5D binding moiety of the bispecific or multi-specific antibodies provided herein comprises a HCDR1, HCDR2 and/or HCDR3 contained within any one of the heavy chain variable region sequences as set forth in Table 3 above, and a LCDR1, LCDR2 and/or LCDR3 contained within any one of the light chain variable region sequences as set forth in Table 3 above. In certain embodiments, the GPRC5D binding moiety of the bispecific or multi-specific antibodies provided herein comprises a HCDR1, HCDR2 and/or HCDR3 comprising an amino acid sequence as set forth in Table 2 above. In certain embodiments, the GPRC5D binding moiety of the bispecific or multi-specific antibodies provided herein comprises a LCDR1, LCDR2 and/or LCDR3 comprising an amino acid sequence as set forth in Table 2 above. In certain embodiments, the GPRC5D binding moiety of the bispecific or multi-specific antibodies provided herein comprises HCDR1, HCDR2, HCDR3, LCDR1, LCDR2 and LCDR3 comprising an amino acid sequence as set forth in Table 2 above. In certain embodiments, the GPRC5D binding moiety of the bispecific or multi-specific antibodies provided herein comprises a HCDR1, HCDR2, and/or HCDR3 derived from the illustrative anti-GPRC5D antibody 36A3G8, 28H9C12, 26E2H6, 20C4E1, 15D6G2, 12G12C2, 71F8H2, 64G9C1, 98A3A8, 96C9E7, 90G6H3, 89FI 1E3, 87A3G8, 83A9B8, 82A1C10, 119E4134, 116G1G4, 116F1G6, 110B4H5, 108C4C12 or 101B3G3 as described above. In certain embodiments, the GPRC5D binding moiety of the bispecific or multi-specific antibodies provided herein comprises HCDR1, HCDR2, HCDR3, LCDR1, LCDR2 and LCDR3 derived from the illustrative anti-GPRC5D antibody 36A3G8, 28H9C12, 26E2H6, 20C4E1, 15D6G2, 12G12C2, 71F8H2, 64G9C1, 98A3A8, 96C9E7, 90G6H3, 89F11E3, 87A3G8, 83A9B8, 82A1C10, 119E4B4, 116G1G4, 116F1G6, 110B4H5, 108C4C12 or 101B3G3 as described above.

In certain embodiments, the GPRC5D binding moiety of the bispecific or multi-specific antibodies provided herein comprises a heavy chain variable region having an amino acid sequence as set forth in Table 3 above, or a homologous sequence having at least 80% (e.g., at least 85%, at least 90%, at least 91%, at least 92%, at least 93%, at least 94%, at least 95%, at least 96%, at least 97%, at least 98%, and at least 99%) sequence identity to the amino acid sequence as set forth in Table 3 above. In certain embodiments, the GPRC5D binding moiety of the bispecific or multi-specific antibodies provided herein comprises a light chain variable region having an amino acid sequence as set forth in Table 3 above, or a homologous sequence having at least 80% (e.g., at least 85%, at least 90%, at least 91%, at least 92%, at least 93%, at least 94%, at least 95%, at least 96%, at least 97%, at least 98%, and at least 99%) sequence identity to the amino acid sequence as set forth in Table 3 above. In certain embodiments, the GPRC5D binding moiety of the bispecific or multi-specific antibodies provided herein comprises a VH/VL amino acid sequence pair selected from the group consisting of SEQ ID NOs: 7/8, 15/16, 23/24, 31/32, 39/40, 47/48, 55/56, 63/64, 71/72, 79/80, 103/104, 111/112, 119/120, 127/128, 135/136, 143/144, 151/152, 159/160, 175/176, 183/184, or 191/192.

As used herein, the term “CD3 binding moiety” when referring to a bispecific or multi-specific antibody or antigen-binding fragment thereof, refers to a moiety that is capable of binding to CD3 (e.g., human CD3, mouse CD3, and cynomolgus CD3). The CD3 binding moiety can take any form that allows specific recognition of the target CD3. For example, the CD3 binding moiety may be an antibody or an antigen-binding fragment thereof, e.g., an IgG (such as IgG1, IgG2, IgG3, and IgG4) antibody, IgA antibody, or IgM antibody. In some embodiments, the CD3 binding moiety of the bispecific or multi-specific antibodies provided herein is derived from an anti-CD3 antibody that binds and activates primary T cells. The CD3 binding moiety of the bispecific or multi-specific antibodies provided herein may be derived from any of the anti-CD3 antibodies known in the art, e.g., the anti-CD3 antibodies described in US20200048348A1, WO2013186613A1, WO2015001085A1, etc.

In some embodiments, the CD3 binding moiety of the bispecific or multi-specific antibodies provided herein comprises one or two or three heavy chain complementarity determining regions (HCDR1, HCDR2 and/or HCDR3) contained within the heavy chain variable (VH) region sequence of SEQ ID NO: 87 or 95; and/or one or two or three light chain complementarity determining regions (LCDR1, LCDR2 and/or LCDR3) contained with the light chain variable (VL) region sequence of SEQ ID NO: 88 or 96. In some embodiments, the CD3 binding moiety of the bispecific or multi-specific antibodies provided herein comprises HCDR1, HCDR2 and/or HCDR3 contained within the VH region sequence of SEQ ID NO: 87, and LCDR1, LCDR2 and/or LCDR3 contained within the VL region sequence of SEQ ID NO: 88. In some embodiments, the CD3 binding moiety of the bispecific or multi-specific antibodies provided herein comprises HCDR1, HCDR2 and/or HCDR3 contained within the VH region sequence of SEQ ID NO: 95, and LCDR1, LCDR2 and/or LCDR3 contained within the VL region sequence of SEQ ID NO: 96. In some embodiments, the CDR boundaries of the CD3 binding moiety are identified by the convention of Kabat. In some embodiments, the CDR boundaries of the CD3 binding moiety are identified by the convention of IMGT. In some embodiments, the CDR boundaries of the CD3 binding moiety are identified by the convention of Chothia. In some embodiments, the CDR boundaries of the CD3 binding moiety are identified by the convention of Al-Lazikani.

In some embodiments, the CD3 binding moiety of the bispecific or multi-specific antibodies provided herein comprises a HCDR1 comprising an amino acid sequence as set forth in SEQ ID NO: 81 or 89, a HCDR2 comprising an amino acid sequence as set forth in SEQ ID NO: 82 or 90, and a HCDR3 comprising an amino acid sequence as set forth in SEQ ID NO: 83 or 91; and/or a LCDR1 comprising an amino acid sequence as set forth in SEQ ID NO: 84 or 92, a LCDR2 comprising an amino acid sequence as set forth in SEQ ID NO: 85 or 93, and a LCDR3 comprising an amino acid sequence as set forth in SEQ ID NO: 86 or 94.

In some embodiments, the CD3 binding moiety of the bispecific or multi-specific antibodies provided herein comprises:
i. a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86; or
ii. a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94.

In some embodiments, the CD3 binding moiety of the bispecific or multi-specific antibodies provided herein comprises a VH/VL amino acid sequence pair of SEQ ID NOs: 87/88 or 95/96.

The amino acid sequences of the CDRs, VH regions and VL regions of illustrative CD3 binding moiety are shown in Table 5 below. The CDR boundaries as described in Table 5 below were defined or identified by the convention of Kabat.

TABLE 5
Amino acid sequences of HCDRs, LCDRs, VH regions and VL regions
of two exemplary CD3 binding moiety

| Sequence | SEQ ID | |
|---|---|---|
| Description | NO | Amino Acid Sequence |
| HCDR1 | 81 | NYAMH |
| HCDR2 | 82 | GISWNSGSIGYADSVKG |
| HCDR3 | 83 | DSRGYGDYSRGGAY |
| LCDR1 | 84 | RASQSVSSNLA |
| LCDR2 | 85 | GASTRAT |
| LCDR3 | 86 | QQYNNWPWT |
| VH | 87 | EVQLVESGGGLVQPGRSLRLSCAASGFTFDNYAMHW |
| | | VRQAPGKGLEWVSGISWNSGSIGYADSVKGRFTISRD |
| | | NAKNSLYLQMNSLRAEDTALYYCAKDSRGYGDYSRG |
| | | GAYWGQGTLVTVSS |
| VL | 88 | EIVMTQSPATLSVSPGERATLSCRASQSVSSNLAWYQQ |
| | | KPGQAPRLLIYGASTRATGIPARFSGSGSGTEFTLTISSL |
| | | QSEDFAVYYCQQYNNWPWTFGQGTKVEIK |
| HCDR1 | 89 | TYAMN |
| HCDR2 | 90 | RIRSKYNNYATYYAASVKG |
| HCDR3 | 91 | HGNFGNSYVSWFAY |
| LCDR1 | 92 | RSSTGAVTTSNYAN |
| LCDR2 | 93 | GTNKRAP |
| LCDR3 | 94 | ALWYSNLWV |
| VH | 95 | EVQLVESGGGLVQPGGSLRLSCAASGFTFNTYAMNW |
| | | VRQAPGKGLEWVARIRSKYNNYATYYAASVKGRFTIS |
| | | RDDSKNSLYLQMNSLKTEDTAVYYCARHGNFGNSYV |
| | | SWFAYWGQGTLVTVSS |
| VL | 96 | QTVVTQEPSLTVSPGGTVTLTCRSSTGAVTTSNYANW |
| | | VQQKPGQAPRGLIGGTNKRAPGTPARFSGSLLGGKAA |
| | | LTLSGVQPEDEAEYYCALWYSNLWVFGGGTKLTVL |

An exemplary bispecific antibody or antigen-binding fragment thereof provided herein comprises:
(1) a GPRC5D binding moiety comprising HCDR1, HCDR2, and HCDR3 contained within VH region sequence as set forth in SEQ ID NO: 127, and LCDR1, LCDR2, and LCDR3 contained within VL region sequence as set forth in SEQ ID NO: 128; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88;
(2) a GPRC5D binding moiety comprising HCDR1, HCDR2, and HCDR3 contained within VH region sequence as set forth in SEQ ID NO: 183, and LCDR1, LCDR2, and LCDR3 contained within VL region sequence as set forth in SEQ ID NO: 184; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88;
(3) a GPRC5D binding moiety comprising HCDR1, HCDR2, and HCDR3 contained within VH region sequence as set forth in SEQ ID NO: 151, and LCDR1, LCDR2, and LCDR3 contained within VL region sequence as set forth in SEQ JD NO: 152; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88;
(4) a GPRC5D binding moiety comprising HCDR1, HCDR2, and HCDR3 contained within VH region sequence as set forth in SEQ ID NO: 159, and LCDR1, LCDR2, and LCDR3 contained within VL region sequence as set forth in SEQ ID NO: 160; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88;
(5) a GPRC5D binding moiety comprising HCDR1, HCDR2, and HCDR3 contained within VH region sequence as set forth in SEQ ID NO: 39, and LCDR1, LCDR2, and LCDR3 contained within VL region sequence as set forth in SEQ ID NO: 40; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88;
(6) a GPRC5D binding moiety comprising HCDR1, HCDR2, and HCDR3 contained within VH region sequence as set forth in SEQ ID NO: 71, and LCDR1, LCDR2, and LCDR3 contained within VL region sequence as set forth in SEQ ID NO: 72; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88;
(7) a GPRC5D binding moiety comprising HCDR1, HCDR2, and HCDR3 contained within VH region sequence as set forth in SEQ ID NO: 79, and LCDR1, LCDR2, and LCDR3 contained within VL region sequence as set forth in SEQ ID NO: 80; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88;
(8) a GPRC5D binding moiety comprising HCDR1, HCDR2, and HCDR3 contained within VH region sequence as set forth in SEQ ID NO: 175, and LCDR1, LCDR2, and LCDR3 contained within VL region sequence as set forth in SEQ ID NO: 176; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88; or
(9) a GPRC5D binding moiety comprising HCDR1, HCDR2, and HCDR3 contained within VH region sequence as set forth in SEQ ID NO: 23, and LCDR1, LCDR2, and LCDR3 contained within VL region sequence as set forth in SEQ ID NO: 24; and a CD3 binding moiety comprising HCDR1, HCDR2, and HCDR3 contained within VH region sequence as set forth in SEQ ID NO: 87, and LCDR1, LCDR2, and LCDR3 contained within VL region sequence as set forth in SEQ ID NO: 88.

Another exemplary bispecific antibody or antigen-binding fragment thereof provided herein comprises:
(1) a GPRC5D binding moiety comprising HCDR1, HCDR2, HCDR3, LCDR1, LCDR2 and LCDR3 as set forth in SEQ ID NOs: 121, 122, 123, 124, 125, and 126, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively;
(2) a GPRC5D binding moiety comprising HCDR1, HCDR2, HCDR3, LCDR1, LCDR2 and LCDR3 as set forth in SEQ ID NOs: 177, 178, 179, 180, 181, and 182, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively;
(3) a GPRC5D binding moiety comprising HCDR1, HCDR2, HCDR3, LCDR1, LCDR2 and LCDR3 as set forth in SEQ ID NOs: 145, 146, 147, 148, 149, and 150, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively;
(4) a GPRC5D binding moiety comprising HCDR1, HCDR2, HCDR3, LCDR1, LCDR2 and LCDR3 as set forth in SEQ ID NOs: 153, 154, 155, 156, 157, and 158, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively;
(5) a GPRC5D binding moiety comprising HCDR1, HCDR2, HCDR3, LCDR1, LCDR2 and LCDR3 as set forth in SEQ ID NOs: 33, 34, 35, 36, 37, and 38, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively;
(6) a GPRC5D binding moiety comprising HCDR1, HCDR2, HCDR3, LCDR1, LCDR2 and LCDR3 as set forth in SEQ ID NOs: 65, 66, 67, 68, 69, and 70, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively;
(7) a GPRC5D binding moiety comprising HCDR1, HCDR2, HCDR3, LCDR1, LCDR2 and LCDR3 as set forth in SEQ ID NOs: 73, 74, 75, 76, 77, and 78, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively;
(8) a GPRC5D binding moiety comprising HCDR1, HCDR2, HCDR3, LCDR1, LCDR2 and LCDR3 as set forth in SEQ ID NOs: 169, 170, 171, 172, 173, and 174, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively; or
(9) a GPRC5D binding moiety comprising HCDR1, HCDR2, HCDR3, LCDR1, LCDR2 and LCDR3 as set forth in SEQ ID NOs: 17, 18, 19, 20, 21, and 22, respectively; and a CD3 binding moiety comprising HCDR1, HCDR2, HCDR3, LCDR1, LCDR2 and LCDR3 as set forth in SEQ ID NOs: 81, 82, 83, 84, 85, and 86, respectively.

Another exemplary bispecific antibody or antigen-binding fragment thereof provided herein comprises:
(1) a GPRC5D binding moiety comprising HCDR1, HCDR2, and HCDR3 contained within VH region sequence as set forth in SEQ ID NO: 183, and LCDR1, LCDR2, and LCDR3 contained within VL region sequence as set forth in SEQ ID NO: 184; and a CD3 binding moiety comprising HCDR1, HCDR2, and HCDR3 contained within VH region sequence as set forth in SEQ ID NO: 95, and LCDR1, LCDR2, and LCDR3 contained within VL region sequence as set forth in SEQ ID NO: 96;
(2) a GPRC5D binding moiety comprising HCDR1, HCDR2, and HCDR3 contained within VH region sequence as set forth in SEQ ID NO: 71, and LCDR1, LCDR2, and LCDR3 contained within VL region sequence as set forth in SEQ ID NO: 72; and a CD3 binding moiety comprising HCDR1, HCDR2, and HCDR3 contained within VH region sequence as set forth in SEQ ID NO: 95, and LCDR1, LCDR2, and LCDR3 contained within VL region sequence as set forth in SEQ ID NO: 96; or
(3) a GPRC5D binding moiety comprising HCDR1, HCDR2, and HCDR3 contained within VH region sequence as set forth in SEQ ID NO: 79, and LCDR1, LCDR2, and LCDR3 contained within VL region sequence as set forth in SEQ ID NO: 80; and a CD3 binding moiety comprising HCDR1, HCDR2, and HCDR3 contained within VH region sequence as set forth in SEQ ID NO: 95, and LCDR1, LCDR2, and LCDR3 contained within VL region sequence as set forth in SEQ ID NO: 96.

Another exemplary bispecific antibody or antigen-binding fragment thereof provided herein comprises:
(1) a GPRC5D binding moiety comprising HCDR1, HCDR2, HCDR3, LCDR1, LCDR2 and LCDR3 as set forth in SEQ ID NOs: 177, 178, 179, 180, 181, and 182, respectively; and a CD3 binding moiety comprising HCDR1, HCDR2, HCDR3, LCDR1, LCDR2 and LCDR3 as set forth in SEQ ID NOs: 89, 90, 91, 92, 93, and 94, respectively;
(2) a GPRC5D binding moiety comprising HCDR1, HCDR2, HCDR3, LCDR1, LCDR2 and LCDR3 as set forth in SEQ ID NOs: 65, 66, 67, 68, 69, and 70, respectively; and a CD3 binding moiety comprising HCDR1, HCDR2, HCDR3, LCDR1, LCDR2 and LCDR3 as set forth in SEQ ID NOs: 89, 90, 91, 92, 93, and 94, respectively; or
(3) a GPRC5D binding moiety comprising HCDR1, HCDR2, HCDR3, LCDR1, LCDR2 and LCDR3 as set forth in SEQ ID NOs: 73, 74, 75, 76, 77, and 78, respectively; and a CD3 binding moiety comprising HCDR1, HCDR2, HCDR3, LCDR1, LCDR2 and LCDR3 as set forth in SEQ ID NOs: 89, 90, 91, 92, 93, and 94, respectively.

Different formats of bispecific or multi-specific antibodies have been described in the art, for example, in Chames and Baty (2009) Curr Opin Drug Disc Dev 12: 276. The bispecific or multi-specific antibodies or antigen-binding fragments thereof provide herein are not limited to any particular bispecific format, and may have all the different formats known in the art.

For example, bispecific antibodies or antigen-binding fragments thereof provide herein may have a typical full-length antibody structure, i.e., an antibody having two different full-length antibody heavy chains and two different full length antibody light chains. A full-length antibody heavy chain includes heavy chain variable region (VH) and constant domains CH1, CH2, CH3 and optionally CH4. A full-length antibody light chain includes light chain variable region (VL) and constant domain CL. For example, the bispecific antibodies or antigen-binding fragments thereof provide herein may comprises a first light chain, a first heavy chain, a second heavy chain, and a second light chain, wherein the first light chain and the first heavy chain are paired to form a first antigen-binding site that binds to GPRC5D, and the second light chain and the second heavy chain are paired to form a second antigen-binding site that binds to CD3. For another example, the bispecific antibodies or antigen-binding fragments thereof provide herein may comprises a first light chain, a first heavy chain, a second heavy chain, and a second light chain, wherein the first light chain and the first heavy chain are paired to form a first antigen-binding site that binds to CD3, and the second light chain and the second heavy chain are paired to form a second antigen-binding site that binds to GPRC5D.

For another example, the bispecific or multi-specific antibodies or antigen-binding fragments thereof provide herein may include, but are not limited to, bispecific antibodies with complementary CH3 domains to force heterodimerization, Knobs-into-Holes molecules (Genentech, WO9850431), CrossMAbs (Roche, WO2009080253), or electrostatically-matched molecules (Amgen, EP1870459 and WO2009089004; Chugai, US201000155133; Oncomed, WO2010129304). For example, regarding the CrossMab format, the CD3 binding moiety of the bispecific or multi-specific antibodies provided herein further comprises a constant domain CL and a constant domain CH1, and in some embodiments, the constant domains CL and CH1 are replaced by each other; or the GPRC5D binding moiety of the bispecific or multi-specific antibodies provided herein further comprises a constant domain CL and a constant domain CH1, and in some embodiments, the constant domains CL and CH1 are replaced by each other.

In some embodiments, the bispecific or multi-specific antibodies provided herein further comprise an Fc region. In some embodiments, the bispecific or multi-specific antibodies provided herein further comprise an Fc region of human immunoglobulin (Ig), or optionally an Fc region of human IgG. In some embodiments, the Fc region is derived from human IgG1, IgG2, IgG3, or IgG4.

For another example, the bispecific antibody or antigen-binding fragment thereof provided herein may be a bifunctional fusion protein targeting GPRC5D and CD3, wherein the GPRC5D binding moiety is a full-length antibody, and the CD3 binding moiety is an antigen-binding fragment thereof (e.g., a scFv), wherein the CD3 binding moiety is linked to the GPRC5D binding moiety directly or via a linker. The CD3 binding moiety may be linked to the N-terminal of variable regions of the GPRC5D binding moiety, may be linked to the C-terminal of Fc region of the GPRC5D binding moiety, or may be linked to the C-terminal of CL constant domain of the GPRC5D binding moiety. In some embodiments, the CD3 binding moiety may be or comprise a scFv comprising a VH region linked to a VL region directly or via a linker, and the VH region of the CD3 binding moiety is linked to the GPRC5D binding moiety directly or via a linker. In some embodiments, the CD3 binding moiety may be or comprise a scFv comprising a VH region linked to a VL region directly or via a linker, and the VL region of the CD3 binding moiety is linked to the GPRC5D binding moiety directly or via a linker.

For another example, the bispecific antibody or antigen-binding fragment thereof provided herein may be a bifunctional fusion protein targeting GPRC5D and CD3, wherein the CD3 binding moiety is a full-length antibody, and the GPRC5D binding moiety is an antigen-binding fragment thereof (e.g., a scFv), wherein the GPRC5D binding moiety is linked to the CD3 binding moiety directly or via a linker. The GPRC5D binding moiety may be linked to the N-terminal of variable regions of the CD3 binding moiety, may be linked to the C-terminal of Fc region of the CD3 binding moiety, or may be linked to the C-terminal of CL constant domain of the CD3 binding moiety. In some embodiments, the GPRC5D binding moiety may be or comprise a scFv comprising a VH region linked to a VL region directly or via a linker, and the VH region of the GPRC5D binding moiety is linked to the CD3 binding moiety directly or via a linker. In some embodiments, the GPRC5D binding moiety may be or comprise a scFv comprising a VH region linked to a VL region directly or via a linker, and the VL region of the GPRC5D binding moiety is linked to the CD3 binding moiety directly or via a linker.

The term “linker” as used herein refers to an artificial amino acid sequence having 1, 2, 3, 4 or 5 amino acid residues, or a length of between 5 and 15, 20, 30, 50 or more amino acid residues, joined by peptide bonds and are used to link one or more polypeptides. A linker may or may not have a secondary structure. Linker sequences are known in the art, see, for example, Holliger et at, Proc. Natl. Acad. Sci. USA 90:6444-6448 (1993); Poljak et al., Structure 2:1121-1123 (1994).

In certain embodiments, the linker is selected from the group consisting of a cleavable linker, a non-cleavable linker, a peptide linker, a flexible linker, a rigid linker, a helical linker, and a non-helical linker. Any suitable linkers known in the art can be used. In certain embodiments, the linker comprises a peptide linker. For example, a useful linker in the present disclosure may be rich in glycine and serine residues. Examples include linkers having a single or repeated sequences comprising threonine/serine and glycine, such as TGGGG (SEQ ID NO: 164), GGGGS (SEQ ID NO: 165) or SGGGG (SEQ ID NO: 166) or its tandem repeats (e.g., 2, 3, 4, 5, 6, 7, 8, 9, 10 or more repeats). In certain embodiments, the linker used in the present disclosure comprises GGGGSGGGGSGGGGS (SEQ ID NO: 167). In certain embodiments, the GS linker comprises one or more repeats of GGGS (SEQ ID NO: 168). In certain embodiments, the first linker comprises or consists of an amino acid sequence having at least 80%, at least 85%, at least 90%, at least 91%, at least 92%, at least 93%, at least 94%, at least 95%, at least 96%, at least 97%, at least 98%, at least 99% sequence identity to any one of SEQ ID NOs: 164-168.

### Conjugates

In some embodiments, the anti-GPRC5D antibodies or antigen-binding fragments thereof provided herein further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding (e.g., disulfide bond), affinity binding, intercalation, coordinate binding, complexation, association, blending, or addition, among other methods. In some embodiments, the antibodies or antigen-binding fragments thereof can be linked to one or more conjugates via a linker or a crosslinking agent. The linker or crosslinking agent comprises a reactive chemical group that can react with the anti-GPRC5D antibodies or fragments thereof. The reactive chemical groups can be N-succinimidyl esters and N-sulfosuccinimidyl esters. Additionally, the linker comprises a reactive chemical group, which can be a dithiopyridyl group that can react with the drug to form a disulfide bond. Linker molecules include, for example, N-succinimidyl 4-(maleimidomethyl) cyclohexanecarboxylate (SMCC), N-succinimidyl 3-(2-pyridyldithio) propionate (SPDP) (see, e.g., Carlsson et al, Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)2-sulfobutanoate (sulfo-SPDB) (see US Publication No. 20090274713), N-succinimidyl 4-(2-pyridyldithio) pentanoate (SPP) (see, e.g., CAS Registry number 341498-08-6), 2-iminothiolane, or acetylsuccinic anhydride. For example, the antibody or cell binding agent can be modified with crosslinking reagents and the antibody or cell binding agent containing free or protected thiol groups thus derived is then reacted with a disulfide- or thiol-containing maytansinoid to produce conjugates. The conjugates can be purified by chromatography, including but not limited to HPLC, size-exclusion, adsorption, ion exchange and affinity capture, dialysis or tangential flow filtration.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies or antigen-binding fragments thereof provided herein may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g., a polymer such as PEG which extends half-life), a chemotherapeutic agent, a toxin, a radioactive isotope, a lanthanide, a detectable label (e.g., a luminescent label, a fluorescent label, an enzyme-substrate label), a DNA-alkylator, a topoisomerase inhibitor, a tubulin-binder, a purification moiety or other anticancer drugs (e.g., agonist of toll-like receptor 7 (TLR-7), TLR-8 and/or TLR-9, siRNA, antibody or antigen-binding fragments thereof, a peptide (such as a short peptide), etc.).

A “toxin” can be any agent that is detrimental to cells or that can damage or kill cells. Examples of toxin include, without limitation, taxol, taxoids, CC-1065 and CC-1065 analogs, duocarmycins and duocarmycin analogs, enediynes such as calicheamicins, dolastatin and dolastatin analogs including auristatins, tomaymycin derivatives, leptomycin derivatives, cisplatin, carboplatin, daunorubicin, doxorubicin, vincristine, vinblastine, melphalan, mitomycin C, chlorambucil and morpholino doxorubicin, cytochalasin B, gramicidin D, ethidium bromide, emetine, mitomycin, etoposide, tenoposide, vincristine, MMAE, MMAF, DM1, DM4, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechloretharnine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent label (e.g., fluorescein, rhodamine, dansyl, phycoerythrin, or Texas Red), an enzyme-substrate label (e.g., horseradish peroxidase, alkaline phosphatase, luceriferases, glucoamylase, lysozyme, saccharide oxidases or β-D-galactosidase), a radioisotope (e.g., ¹²³I, ¹²⁴I, ¹²⁵I ¹³¹I ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), a luminescent label, a chromophoric moiety, digoxigenin, biotin/avidin, a DNA molecule or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibody or an antigen-binding fragment thereof provided herein is used as a base for a conjugate.

In certain embodiments, the antibody or an antigen-binding fragment thereof provided herein is conjugated to a signal peptide. A signal peptide (sometimes referred to as signal sequence, leader sequence or leader peptide) can be used to facilitate secretion and isolation of the antibodies or antigen-binding fragments thereof provided herein. Signal peptides are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

### Chimeric Antigen Receptor

In certain embodiments, the present disclosure provides a chimeric antigen receptor comprising the antibody or an antigen-binding fragment thereof provided herein, a transmembrane region and an intracellular signal region.

The term “chimeric antigen receptor” or “CAR” or “CARs” as used herein refers to engineered receptors, which graft an antigen specificity onto cells (for example, T cells such as naive T cells, central memory T cells, effector memory T cells, regulatory T cells or combination thereof). CARs are also known as artificial T-cell receptors, chimeric T-cell receptors or chimeric immunoreceptors. In some embodiments, CARs comprise an antigen-specific targeting region (for example, the antigen-binding fragments of the anti-GPRC5D antibody as provided herein), an extracellular region, a transmembrane region, one or more co-stimulatory regions, and an intracellular signal region.

In some embodiments, the antigen-specific targeting region is an scFv. In some embodiments, the transmembrane region comprises a transmembrane region of CD3, CD4, CD8 or CD28. In some embodiments, the co-stimulatory region comprises a co-stimulatory domain of CD28, ICOS, CD27, 4-11B, OX40 and CD40L. In some embodiments, the intracellular signal region is selected from the group consisting of: an intracellular signal region sequence of CD3 (e.g. CD3ζ), FcγRI, CD27, CD28, CD137, CD134, MyD88, CD40, CD278, TLRs, or a combination thereof. In some embodiments, the CD8 transmembrane region comprises an amino acid sequence as set forth in SEQ ID NO: 203. In some embodiments, the 4-1BB intracellular signal region comprises an amino acid sequence as set forth in SEQ ID NO: 204. In some embodiments, the CD3ζ intracellular signal region comprises an amino acid sequence as set forth in SEQ ID NO: 205. In some embodiments, the intracellular signal region comprises a 4-1BB intracellular signal region comprising an amino acid sequence as set forth in SEQ ID NO: 204, and a CD3ζ intracellular signal region comprising an amino acid sequence as set forth in SEQ ID NO: 205.

The CARs may be grafted onto various cells, for example, allogeneic cells, autologous cells or xenogeneic cells.

The term “allogeneic cell” as used herein refers to any cells derived from a different subject of the same species.

The term “autologous cell” as used herein refers to any cells derived from the same subject into which they are later to be re-introduced.

The term “xenogeneic cell” as used herein refers to any cells derived from a different subject of a different species.

In some embodiments, the CARs are grafted on immune effector cells, for example, T cells, natural killer cells, macrophage cells, tumor-infiltrating lymphocytes, etc.

In some embodiments, the CARs provided herein exhibit comparable or better cytotoxicity compared to known CARs targeting GPRC5D in the art, for example, those disclosed in WO2020092854A2. In some embodiments, the CARs provided herein exhibit comparable or better cytotoxicity compared to BMK-203. The term “BMK-203” comprises a scFv targeting GPRC5D, a CD8 transmembrane region, a 4-1BB intracellular signal region and a CD3 ζ intracellular signal region, wherein the scFv targeting GPRC5D comprises a VH region comprising an amino acid sequence as set forth in SEQ ID NO: 201 and a VL region comprising an amino acid sequence as set forth in SEQ ID NO: 202; the CD8 transmembrane region comprises an amino acid sequence as set forth in SEQ ID NO: 203; the 4-1BB intracellular signal region comprises an amino acid sequence as set forth in SEQ ID NO: 204; and the CD3ζ intracellular signal region comprises an amino acid sequence as set forth in SEQ ID NO: 205. The amino acid sequences of SEQ ID NOs: 201, 202, 203, 204 and 205 are shown below.

| Sequence | SEQ ID | |
|---|---|---|
| Description | NO | Amino Acid Sequence |
| BMK-203 VH (the | 201 | QVQLVESGGGLVHPGGSLRLSCAASGFTFRSHSM |
| CDR sequences are | | NWVRQAPGKGLEWVSSISSDSTYTYYADSVKGRF |
| bold and underlined) | | TISRDNAKNSLYLQMNSLRAEDTAVYYCARSGGQ |
| | | WKYYDYWGQGTLVTVSS |
| BMK-203 VL (the | 202 | SSELTQDPAVSVALGQTVRITCQGDSLRSYYASW |
| CDR sequences are | | YQQKPGQAPVLVIYGKNNRPSGIPDRFSGSSSGN |
| bold and underlined) | | TASLTITGAQAEDEADYYCNSRDSSGNPPVVFGG |
| | | GTKLTVL |
| CD8 transmembrane | 203 | TTTPAPRPPTPAPTIASQPLSLRPEACRPAAGGA |
| region | | VHTRGLDFACDIYIWAPLAGTCGVLLLSLVITLY |
| | | C |
| 4-1BB intracellular | 204 | KRGRKKLLYIFKQPFMRPVQTTQEEDGCSCRFP |
| signal region | | EEEEGGCEL |
| CD32 intracellular | 205 | RVKFSRSADAPAYKQGQNQLYNELNLGRREEYD |
| signal region | | VLDKRRGRDPEMGGKPRRKNPQEGLYNELQKDK |
| | | MAEAYSEIGMKGERRRGKGHDGLYQGLSTATKD |
| | | TYDALHMQALPPR |

### Polynucleotides and Recombinant Methods

The present disclosure provides isolated polynucleotides that encode the antibodies or antigen-binding fragments thereof, and/or the chimeric antigen receptors provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the antibody or antigen-binding fragment thereof provided herein is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the antibodies or antigen-binding fragments thereof and/or the chimeric antigen receptors provided herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence.

The present disclosure provides vectors comprising the isolated polynucleotides provided herein. In certain embodiments, the polynucleotides provided herein encodes the antibodies or antigen-binding fragments thereof and/or the chimeric antigen receptors provided herein, at least one promoter (e.g., SV40, CMV, EF-Iα) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pC1, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof and/or the chimeric antigen receptors provided herein can be introduced to a host expression system (e.g., a host cell) for cloning or gene expression. In certain embodiments, the host expression system provided herein is a microorganism, a yeast, or a mammalian cell. In certain embodiments, the microorganism is selected from the group consisting of E. coli and B. subtilis. In certain embodiments, the yeast is Saccharomyces. In certain embodiments, the mammalian cell is selected from the group consisting of COS, CHO-S, CHO-K1, HEK-293, and 3T3 cells.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-GPRC5D antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; mouse forestomach carcinoma cells (MFC), SNU620 cells, and a human hepatoma line (Hep G2). In some embodiments, the host cell is a mammalian cultured cell line, such as CHO, BHK, NS0, 293, MFC, SNU620 and their derivatives.

Host cells are transformed with the above-described expression or cloning vectors for antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or antigen-binding fragment thereof and/or the chimeric antigen receptors provided herein, comprising culturing the host expression system provided herein under the condition at which the antibody or antigen-binding fragment thereof and/or the chimeric antigen receptor is expressed. The host expression systems used to produce the antibodies or antigen-binding fragments thereof and/or the chimeric antigen receptors provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM) (Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibodies or antigen-binding fragments thereof and/or the chimeric antigen receptors prepared from the host expression systems can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof and/or the chimeric antigen receptors. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Composition

The present disclosure further provides pharmaceutical compositions comprising the antibodies or antigen-binding fragments thereof and/or the chimeric antigen receptors provided herein and one or more pharmaceutically acceptable carriers.

Pharmaceutical acceptable carriers for use in the pharmaceutical compositions disclosed herein may include, for example, pharmaceutically acceptable liquid, gels, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides a kit comprising the antibody or an antigen-binding fragment thereof provided herein and/or the chimeric antigen receptors provided herein and/or the pharmaceutical composition provided herein. In certain embodiments, the present disclosure provides a kit comprising the antibody or an antigen-binding fragment thereof provided herein and/or the chimeric antigen receptors provided herein and/or the pharmaceutical composition provided herein, and a second therapeutic agent. In certain embodiments, the second therapeutic agent is selected from the group consisting of a chemotherapeutic agent, an anti-cancer drug, radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Methods of Use

The present disclosure also provides methods of treating, preventing or alleviating a disease, disorder or condition in a subject, comprising administering to the subject a therapeutically effective amount of the antibody or antigen-binding fragment thereof provided herein, and/or the chimeric antigen receptors provided herein, and/or the pharmaceutical composition provided herein. In certain embodiments, the disease, disorder or condition is a GPRC5D-related disease, disorder or condition. In certain embodiments, the subject is human.

In some embodiments, the GPRC5D-related disease, disorder or condition is characterized in expressing or over-expressing of GPRC5D.

In certain embodiments, the disease, disorder or condition is cancer. In certain embodiments, the cancer is a GPRC5D-expressing cancer. “GPRC5D-expressing” cancer as used herein refers to a cancer characterized in expressing GPRC5D protein in a cancer cell, a tumor infiltrating immune cell, or expressing GPRC5D in a cancer cell, a tumor infiltrating immune cell at a level significantly higher than that would have been expected of a normal cell. Various methods can be used to determine the presence and/or amount of GPRC5D in a test biological sample from the subject. For example, the test biological sample can be exposed to an anti-GPRC5D antibody or antigen-binding fragment thereof, which binds to and detects the expressed GPRC5D protein. Alternatively, GPRC5D can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor). In certain embodiments, the cancer is a solid tumor or hematologic tumor. In certain embodiments, the cancer is a GPRC5D-expressing B cell cancer.

In certain embodiments, the disease, disorder or condition is selected from the group consisting of lung cancer (e.g., non-small-cell lung cancer (NSCLC), small cell lung cancer (SCLC), adenocarcinoma of the lung, or squamous cell carcinoma of the lung), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), cutaneous or intraocular melanoma, thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma. In some embodiments, the disease, disorder or condition is multiple myeloma.

In some embodiments, the subject has been identified as having a cancer cell or tumor infiltrating immune cells expressing GPRC5D, optionally at a level significantly higher from the level normally found on non-cancer cells.

In another aspect, methods are provided to treat, prevent or alleviate a disease, disorder or condition in a subject that would benefit from modulation of GPRC5D activity, comprising administering to the subject a therapeutically effective amount of the antibody or antigen-binding fragment thereof provided herein, and/or the pharmaceutical composition provided herein, and/or the chimeric antigen receptor provided herein. In certain embodiments, the disease, disorder or condition is a GPRC5D-related disease, disorder or condition, which is defined above.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g., physician or veterinarian) as indicated by these and other circumstances or requirements.

In certain embodiments, the antibody or antigen-binding fragment provided herein and/or the chimeric antigen receptors provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies or antigen-binding fragments thereof provided herein and/or the chimeric antigen receptors provided herein may be administered by any route known in the art, for example the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

In some embodiments, the antibodies or antigen-binding fragments thereof provided herein and/or the chimeric antigen receptors provided herein may be administered alone or in combination with a therapeutically effective amount of a second therapeutic agent. For example, the antibodies or antigen-binding fragments thereof disclosed herein and/or the chimeric antigen receptors provided herein may be administered in combination with a second therapeutic agent, for example, a chemotherapeutic agent, an anti-cancer drug, a radiation therapy agent, an immunotherapy agent, a targeted therapy agent, a cellular therapy agent, a gene therapy agent, a hormonal therapy agent, an antiviral agent, an antibiotic, an analgesics, an antioxidant, a metal chelator, cytokines, an active agent, an imaging agent, a cytotoxic agent, an angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, or a microRNA.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In certain of these embodiments, an antibody or antigen-binding fragment thereof provided herein, and/or the chimeric antigen receptors provided herein, and/or the pharmaceutical composition provided herein that is administered in combination with one or more additional therapeutic agents may be administered simultaneously with the one or more additional therapeutic agents, and in certain of these embodiments the antibody or antigen-binding fragment thereof and/or the pharmaceutical composition provided herein, and the additional therapeutic agent(s) may be administered as part of the same pharmaceutical composition. However, an antibody or antigen-binding fragment thereof and/or the chimeric antigen receptors provided herein and/or a pharmaceutical composition provided herein administered “in combination” with another therapeutic agent does not have to be administered simultaneously with or in the same composition as the agent. An antibody or antigen-binding fragment thereof, or a chimeric antigen receptor or pharmaceutical composition administered prior to or after another agent is considered to be administered “in combination” with that agent as the phrase is used herein, even if the antibody or antigen-binding fragment, or the pharmaceutical composition or the chimeric antigen receptor, and the second agent are administered via different routes. Where possible, additional therapeutic agents administered in combination with the antibodies or antigen-binding fragments thereof, chimeric antigen receptors or pharmaceutical compositions disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides method of inactivating GPRC5D-expressing cells in vivo or in vitro, comprising contacting the GPRC5D-expressing cells with the antibody or antigen-binding fragment thereof provided herein, and/or the chimeric antigen receptors provided herein, and/or the pharmaceutical composition provided herein.

The present disclosure further provides methods of modulating GPRC5D activity in GPRC5D-expressing cells, comprising exposing the GPRC5D-expressing cells to the antibodies or antigen-binding fragments thereof provided herein, and/or the chimeric antigen receptors provided herein, and/or the pharmaceutical composition provided herein.

In another aspect, the present disclosure provides methods of detecting the presence or amount of GPRC5D in a sample, comprising contacting the sample with the antibody or antigen-binding fragment thereof provided herein, and/or the chimeric antigen receptors provided herein, and/or the pharmaceutical composition provided herein, and determining the presence or the amount of GPRC5D in the sample.

In another aspect, the present disclosure provides a method of diagnosing a GPRC5D-related disease, disorder or condition in a subject, comprising: a) obtaining a sample from the subject, b) contacting the sample obtained from the subject with the antibody or an antigen-binding fragment thereof provided herein and/or the chimeric antigen receptors provided herein and/or the pharmaceutical composition provided herein; c) determining the presence or amount of GPRC5D in the sample; and d) correlating the presence or the amount of GPRC5D to existence or status of the GPRC5D-related disease, disorder or condition in the subject.

In another aspect, the present disclosure provides kits comprising the antibody or antigen-binding fragment thereof provided herein and/or the chimeric antigen receptors provided herein and/or the pharmaceutical composition provided herein, optionally conjugated with a detectable moiety, which is useful in detecting GPRC5D, optionally recombinant GPRC5D, GPRC5D expressed on cell surface, or GPRC5D-expressing cells. The kits may further comprise instructions for use.

In another aspect, the present disclosure also provides use of the antibody or antigen-binding fragment thereof provided herein and/or the chimeric antigen receptors provided herein and/or the pharmaceutical composition provided herein in the manufacture of a medicament for treating, preventing or alleviating a GPRC5D-related disease, disorder or condition in a subject, in the manufacture of a diagnostic reagent for diagnosing a GPRC5D-related disease, disorder or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Antibody Generation

### 1.1 Immunization

To generate antibodies to GPRC5D, Balb/c and SJL mice were immunized with cells (i.e., human GPRC5D overexpression HEK293 cells (HEK293-hGPRC5D) or OPM2 cells, referred to as “cell immunization”) or genetic material (i.e., full-length human GPRC5D expression DNA construct pTT5-H5 (human GPRC5D), referred to as “genetic immunization”), or GPRC5D protein (i.e., recombinant human GPRC5D virus like particle (hGPRC5D VLP), referred to as “protein immunization”) in each group (as shown in Table 6 below). The primary immunization was followed by several boosts until animals developed satisfactory antiserum titers suitable for hybridoma development. All the immunization strategies were carried out in parallel in order to compare the performance and immune response in serum level. The immunization protocols for each group of animals were shown in Table 6 below.

TABLE 6
Grouping of Animals

| | | | Animal/ | Group | | |
|---|---|---|---|---|---|---|
| Group | Immunogen | Route | Strain | size | Dosage | |
| 1 | HEK293- | I.P. | SJL | 5 | 0.5~1 × 10⁷ | cells |
| | hGPRC5D | | | | | |
| 2 | HEK293- | I.P. | Balb/c | 5 | 0.5~1 × 10⁷ | cells |
| | hGPRC5D | | | | | |
| 3 | pTT5-H5 (human | Gene | SJL | 5 | 4 | μg |
| | GPRC5D) | gun | | | | |
| 4 | pTT5-H5 (human | Gene | Balb/c | 5 | 4 | μg |
| | GPRC5D) | gun | | | | |
| 5 | hGPRC5D VLP | I.P. | SJL | 5 | 25~50 | μg |
| 6 | hGPRC5D VLP | I.P. | Balb/c | 5 | 25~50 | μg |

TABLE 7
Immunization protocol for Group 1&2

| | |
|---|---|
| Day 0 | Pre-bleed (15~30 μL serum/mouse) |
| | Primary: 0.5~1 × 10⁷ HEK293-hGPRC5D/mouse I.P. |
| Day 14 | Boost 1: 0.5~1 × 10⁷ HEK293-hGPRC5D cell/mouse I.P. |
| Day 21 | Test bleed (15-30 μL serum/mouse) (TB1) |
| Day 22 | Test bleed FACS |
| Day 35 | Boost 2: 0.5~1 × 10⁷ HEK293-hGPRC5D cell/mouse I.P. |
| Day 42 | Test bleed (15~30 μL serum/mouse) (TB2) |
| Day 43 | Test bleed FACS |
| Day 44 | Data analysis and phase conclusion |
| Day 56 | Pre-fusion (final) boost, 0.5~1 × 10⁷ OPM2 cells/mouse I.P. |

Animals not selected for cell fusion were maintained in cage and given additional boost immunizations.

TABLE 8
Immunization protocol for Group 3&4

| | |
|---|---|
| Day 0 | Pre-bleed (15~30 μL serum/mouse) |
| | Primary immunization, 1 μg/shot, 4 shots/animal, granulocyte- |
| | macrophage colony-stimulating factor (GM-CSF) and FMS-like |
| | tyrosine kinase 3 ligand (FLT3L) |
| Day 14 | Boost 1: 1 μg/shot, 4 shots/animal, GM-CSF&FLT3L |
| Day 21 | Test bleed (15~30 μL serum/mouse) |
| Day 22 | Test bleed FACS |
| Day 28 | Boost 2: 1 μg/shot, 4 shots/animal, GM-CSF&FLT3L |
| Day 35 | Test bleed (15~30 μL serum/mouse) |
| Day 36 | Test bleed FACS |
| Day 42 | Test bleed (15~30 μL serum/mouse) |
| Day 43 | Test bleed FACS |
| Day 44 | Data analysis and phase conclusion |
| Day 56 | Pre-fusion (final) Boost: 0.5~1 × 10⁷ HEK293- |
| | hGPRC5D/mouse |

Animals not selected for cell fusion were maintained in cage and given additional boost immunizations.

TABLE 9
Immunization protocol for Group 5&6

| | |
|---|---|
| Day 0 | Pre-bleed (15~30 μL serum/mouse) |
| | Primary: 50 μg/mouse I.P. |
| Day 14 | Boost 1: 25 μg/mouse I.P. |
| Day 21 | Test bleed (15~30 μL serum/mouse) (TB1) |
| Day 22 | Test bleed FACS |
| Day 35 | Boost 2: 25 μg/mouse I.P. |
| Day 42 | Test bleed (15~30 μL serum/mouse) (TB2) |
| Day 43 | Test bleed FACS |
| Day 44 | Data analysis and phase conclusion |
| Day 56 | Pre-fusion (final) Boost, 25 μg/mouse I.P. |

Animals not selected for cell fusion were maintained in cage and given additional boost immunizations.

### 1.2 Hybridoma Generation and Screening

Splenocyte fusions were performed on the mice which responded the best to the immunizations as determined by test bleed FACS. The lymphocytes from spleens and lymph nodes were fused to a mouse myeloma cell line (Sp2/0-Ag14) using an optimized electrofusion protocol. Multiple fusions were performed to ensure success of the project.

The fusions were plated (1×10⁴ to 10⁵ cells per well) into a stack of 96-well plates. Plates were monitored for growth and fed weekly. Wells with cell growth were screened by primary screening assays in 10-14 days with Mirrorball assay and/or other feasible assays. Multiple fusions for each targeting antigen were performed and screened. The positive parental clones which showed positive binding with HEK293-hGPRC5D from primary screening were expanded into 24-well plates for secondary screening. Following primary screening, positive parental clones expanded into 24-well plates were screened again by the assay described in the hybridoma screening funnel below. Hybridomas of interest were chosen to proceed to subcloning.

The parental hybridomas with desired reactivity and isotypes from the screening funnel above were then subcloned by multiple rounds of limiting dilution or single cell sorting until monoclonals were obtained.

The subcloning plates were screened by Mirrorball assay and the subclones with good binding ability were expanded to 24-wells for confirmation tests. The specificity and cross-reactivity of these subclones were confirmed with FACS analysis. Briefly, parental HEK293T, HEK293T cells stably over-expressing human GPRC5D protein (i.e., HEK293T-hGPRC5D), HEK293T cells stably over-expressing cynomolgus monkey GPRC5D protein (i.e., HEK293T-cynoGPRC5D), HEK293T cells stably over-expressing mouse GPRC5D protein (i.e., HEK293T-mGPRC5D), and OPM2 cells were incubated with antibodies produced by each subclone, respectively. Fluorescent dye-conjugated secondary antibody was used to detect the binding of the primary antibody with the cells. Median fluorescence intensity (MFI) was measured by FACS analysis,

The desired subclonal cell lines were sequenced and further expanded into culture flasks for cryopreservation. 4˜6 vials per cell line at 0.5˜1.0×10⁷ cells/vial were initially cryopreserved. Master cell bank and working cell bank could be established for the selected most valuable cell lines if desired.

As a result, 21 antibodies were discovered with unique sequences showing positive binding with HEK293T-hGPRC5D and OPM2 cells. Among which, some clones could also bind with HEK293T-cynoGPRC5D and/or HEK293T-mGPRC5D. The MFI and positive percentage of the antibodies staining OPM2 cells, parental HEK293T, HEK293T-hGPRC5D, HEK293T-cynoGPRC5D and HEK293T-mGPRC5D detected by FACS were summarized in Table 10A, Table 10B and Table 10C below, suggesting these antibodies were GPRC5D recognizing antibodies. ATG596 was a positive human IgG1 control antibody which comprises a VH region having an amino acid sequence as set forth in SEQ ID NO: 199, and a VL region having an amino acid sequence as set forth in SEQ ID NO: 200.

TABLE 10A
FACS MFI and % positive of antibodies binding with different GPRC5D
expressing cell lines (antibodies produced by protein immunization)

| | Cell line | | | | | | | | | |
|---|---|---|---|---|---|---|---|---|---|---|
| | OPM-2 | | HEK293T | | hGPRC5D | | cynoGPRC5D | | mGPRC5D | |
| Clone NO. | MFI | % Positive | MFI | % Positive | MFI | % Positive | MFI | % Positive | MFI | % Positive |
| Blank | 102.5 | 0.2 | 104.9 | 0.5 | 102.5 | 0.4 | 105.5 | 0.6 | 112.9 | 0.8 |
| ATG596 | 639.9 | 85.9 | 112.2 | 0.9 | 42891.2 | 99.9 | 7399.9 | 98.5 | 14593.6 | 98.2 |
| 36A3G8 | 791.2 | 96.3 | 103.1 | 1.1 | 31839.6 | 99.3 | 3114.1 | 96.6 | 111.6 | 2.1 |
| 28H9C12 | 1121.2 | 97.9 | 104.9 | 0.5 | 25060 | 99.6 | 17953.5 | 99.8 | 120.8 | 1.6 |
| 26E2H6 | 1149.2 | 97.9 | 110.4 | 0.4 | 27190.8 | 99.8 | 116.5 | 1 | 370.3 | 68.4 |
| 20C4E1 | 2022.8 | 99.5 | 107.4 | 0.5 | 38947.6 | 99.9 | 31343 | 99.2 | 592.3 | 82.7 |
| 15D6G2 | 1737.9 | 99.4 | 112.9 | 1.6 | 32794.8 | 99.5 | 7762.3 | 99.7 | 150.1 | 14.6 |
| 12G12C2 | 1073.6 | 98.4 | 110.4 | 4.2 | 32142.7 | 98.7 | 24703.2 | 96.2 | 7080.6 | 99 |

TABLE 10B
FACS MFI and % positive of antibodies binding with different GPRC5D
expressing cell lines (antibodies produced by cell immunization)

| | Cell line | | | | | | | | | |
|---|---|---|---|---|---|---|---|---|---|---|
| | OPM-2 | | HEK293T | | hGPRC5D | | cynoGPRC5D | | mGPRC5D | |
| Clone NO. | MFI | % Positive | MFI | % Positive | MFI | % Positive | MFI | % Positive | MFI | % Positive |
| Blank | 105.3 | 2.9 | 103.7 | 0.5 | 101.5 | 1.7 | 99.9 | 0.4 | 101.5 | 1.4 |
| ATG596 | 421.2 | 79.7 | 104.2 | 1 | 25916.8 | 98.9 | 5583.1 | 96.6 | 12330.6 | 97.8 |
| 71F8H2 | 7982.8 | 99.7 | 109.1 | 0.6 | 2204.8 | 95.2 | 91.3 | 0.6 | 127.4 | 5.6 |
| 64G9C1 | 7739.8 | 99.8 | 105.3 | 0.4 | 1257.1 | 94.4 | 89.6 | 0.5 | 118.8 | 3.4 |

TABLE 10C
FACS MFI and % positive of antibodies binding with different GPRC5D
expressing cell lines (antibodies produced by genetic immunization)

| | Cell line | | | | | | | | | |
|---|---|---|---|---|---|---|---|---|---|---|
| | OPM-2 | | HEK293T | | hGPRC5D | | cynoGPRC5D | | mGPRC5D | |
| Clone NO. | MFI | % Positive | MFI | % Positive | MFI | % Positive | MFI | % Positive | MFI | % Positive |
| Blank | 105.3 | 2.9 | 103.7 | 0.5 | 101.5 | 1.7 | 99.9 | 0.4 | 101.5 | 1.4 |
| ATG596 | 409.3 | 78.8 | 104.8 | 0.5 | 24997.1 | 99.2 | 6580.2 | 98.6 | 12440 | 97.3 |
| 98A3A8 | 961.7 | 98.7 | 102.6 | 0.2 | 2074.7 | 93.2 | 2074.4 | 95.3 | 566.2 | 89.4 |
| 96C9E7 | 1926.7 | 99.9 | 110.2 | 2 | 679.9 | 58.8 | 4020.8 | 71.7 | 202 | 49.2 |
| 90G6H3 | 711.7 | 97.3 | 106.4 | 0.4 | 4941 | 96.7 | 11399.1 | 99.4 | 2231.6 | 87.4 |
| 89F11E3 | 999 | 98.1 | 99.4 | 0.3 | 19227.8 | 96.7 | 16499.2 | 98.8 | 1100.3 | 74.6 |
| 87A3G8 | 450.9 | 92.7 | 100.4 | 0.4 | 7376.4 | 88.6 | 3964.4 | 76.7 | 177.7 | 46.2 |
| 83A9B8 | 823.5 | 98.3 | 112.9 | 1.8 | 4932.9 | 94.1 | 277.8 | 55.7 | 1127.5 | 93.7 |
| 82A1C10 | 550.3 | 94.8 | 114.5 | 0.6 | 8091.9 | 99.4 | 1068.1 | 94.2 | 119.3 | 4.5 |
| 119E4B4 | 867.2 | 98.6 | 109.6 | 0.7 | 3241.6 | 88.2 | 708.8 | 69.6 | 193.9 | 46.5 |
| 116G1G4 | 705.8 | 97.4 | 116.6 | 2.5 | 6051.5 | 94.4 | 344.5 | 59.8 | 838.6 | 84 |
| 116F1G6 | 1036.8 | 98.4 | 112.9 | 0.5 | 42846.8 | 96.2 | 82.6 | 41.4 | 152.3 | 15.7 |
| 110B4H5 | 893.7 | 98.2 | 106.9 | 0.4 | 5249.9 | 98.7 | 2473.2 | 96.7 | 772.2 | 78.9 |
| 108C4C12 | 878.3 | 98.3 | 102.6 | 0.1 | 5013.9 | 96.6 | 355.1 | 59.1 | 273.8 | 65.7 |
| 101B3G3 | 1371.6 | 99.6 | 106.9 | 2.7 | 27051.8 | 97.9 | 584.8 | 57.4 | 1477.4 | 85.2 |

### Example 2. Antibody Characterization: Binding Affinity to Human, Cynomolgus and Mouse GPRC5D

### 2.1 Antibodies

The hybridoma antibody clones 36A3G8, 28H9C12, 26E2H6, 20C4E1, 15D6G2, 12G12C2, 71F8H2, 64G9C1, 98A3A8, 96C9E7, 90G6H3, 89F11E3, 87A3G8, 83A9B8, 82A1C10, 119E4B4, 116G1G4, 116F1G6, 110B4H5, 108C4C12, and 101B3G3 were characterized.

### 2.2 Hybridoma Sequencing

Total RNA was isolated from the hybridoma cells following the technical manual of RNAiso Plus (TAKARA Cat #9109). Total RNA was then reverse transcribed into cDNA using isotype-specific anti-sense primers or universal primers following the technical manual of PrimeScript II 1^{st} Strand cDNA synthesis Kit (TAKARA Cat #6210A). The antibody fragments of VH and VL were amplified according to TaKaRa Taq™ (Cat #R001A). Amplified antibody fragments were cloned into a standard cloning vector separately. Colony PCR was performed to screen for clones with inserts of correct sizes. No less than five colonies with inserts of correct sizes were sequenced for each fragment. The sequences of different clones were aligned and the consensus sequence of these clones was provided. The variable region sequences of the hybridoma antibodies are provided herein in Table 3 above.

### 2.3 Chimeric Antibody Generation

All the sequences of hybridoma antibodies from Table 10A, Table 10B and Table 10C (i.e., 36A3G8, 281H9C12, 26E2H6, 20C4E1, 15D6G2, 12G12C2, 71F8H2, 64G9C1, 98A3A8, 96C9E7, 90G6H3, 89F11E3, 87A3G8, 83A9B8, 82A1C10, 119E4B4, 116G1G4, 116F1G6, 110B4H5, 108C4C12, and 101B3G3) and the benchmark antibody ATG596 were selected to generate and produce human IgG1 chimeric antibodies. DNAs encoding variable regions of the 21 hybridoma antibodies and benchmark antibody ATG596 were synthesized and subcloned into an expression vector where human IgG1 constant gene was included in advance. The vectors were transfected into mammalian cells for recombinant protein expression and the expressed antibody was purified using protein A affinity chromatography column. The resulting chimeric antibodies are referred to herein as ch36A3G8, ch28H9C12, ch26E2H6, ch20C4E1, ch15D6G2, ch12G12C2, ch71F8H2, ch64G9C1, ch98A3A8, ch96C9E7, ch90G6H3, ch89F11E3, ch87A3G8, ch83A9B8, ch82A1C10, ch119E4B4, ch116G1G4, ch116F1G6, ch110B4H5, ch108C4C12, and ch101B3G3, where the prefix “ch” indicates “chimeric”, and the following indicates the hybridoma antibody clone. For example, ch36A3G8 indicates that it is a chimeric antibody derived from the hybridoma antibody clone 36A3G8.

### 2.4 Chimeric Antibody Characterization (Cell Based Binding Affinity to GPRC5D)

The binding affinities of the chimeric antibodies and benchmark antibody ATG596 with CHOS-hGPRC5D, HEK293T-cynoGPRC5D, and HEK293T-mGPRC5D were determined by FACS analysis. The protocol for FACS analysis was described as follows:
(a) Cells were washed by staining buffer (1% BSA in PBS), adjusted cell density with Staining Buffer to 2×10⁵ cells/well.
(b) 50 μL 2× serial diluted test antibodies and cells were added to each well, incubated at 4° C. for 1 hour.
(c) Centrifugated at 300 g for 3 minutes and discarded the supernatant.
(d) Cells were washed by 200 μL staining buffer and repeated step (c).
(e) The cells were incubated 0.5 hours at 4° C. with 100 μL secondary antibody (APC Rat anti-human IgG Fc Antibody, diluted at 1:100) in staining buffer.
(f) Centrifugated at 300 g for 3 minutes and discarded the supernatant.
(g) Cells were washed by 200 μL staining buffer and repeated step (f).
(h) Cells were resuspended with 120 μL staining buffer and analyzed by FACS.

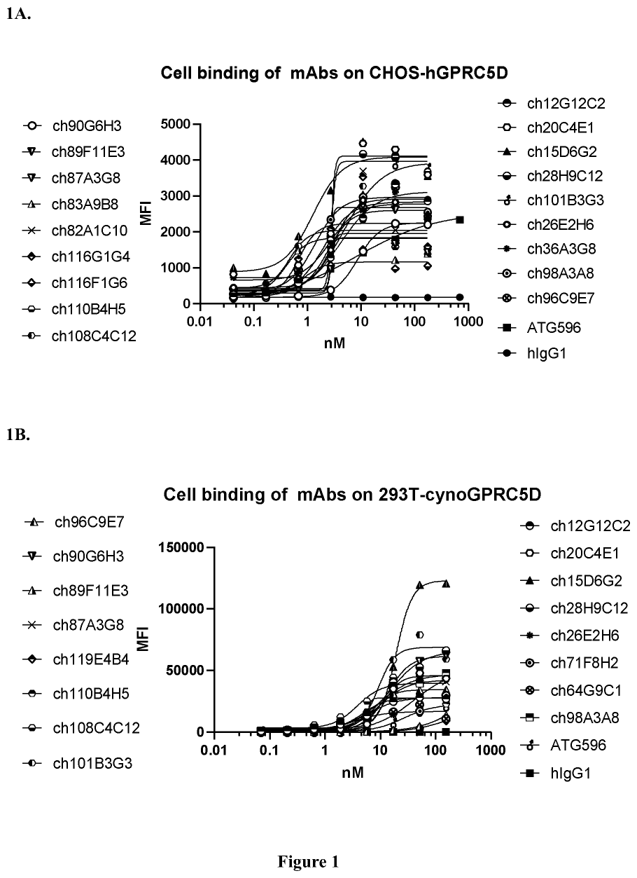

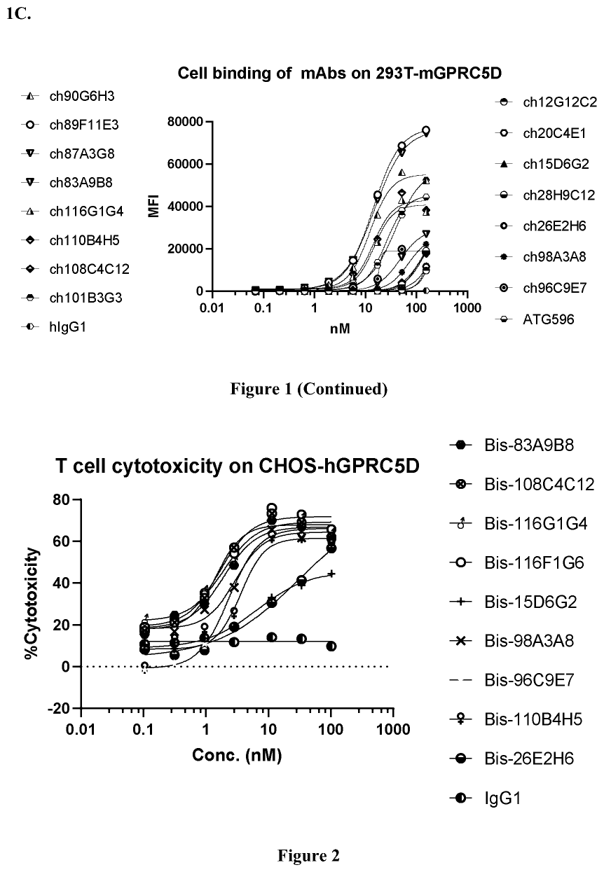

As shown in FIG. 1A, FIG. 1B, FIG. 1C and Table 11 below, 18, 13 and 15 of the chimeric antibodies exhibited positive binding affinity to human, cynomolgus, and murine GPRC5D, respectively.

TABLE 11
Binding affinity of the antibodies to
human, cynomolgus, and mouse GPRC5D

Cell line
Human
CHOS-hGPRC5D

| Clone No. | EC₅₀ (nM) | Top |
|---|---|---|
| hIgG1 | NA | NA |
| ATG596 | 7.857 | 2552 |
| ch12G12C2 | 4.059 | 3120 |
| ch20C4E1 | 2.833 | 4114 |
| ch15D6G2 | 1.273 | 4085 |
| ch28H9C12 | 2.831 | 3964 |
| ch26E2H6 | 3.02 | 2913 |
| ch36A3G8 | 2.641 | 2781 |
| ch98A3A8 | 1.385 | 2966 |
| ch96C9E7 | 1.97 | 2833 |
| ch90G6H3 | 8.513 | 2253 |
| ch89F11E3 | 2.411 | 2601 |
| ch87A3G8 | 2.812 | 1952 |
| ch83A9B8 | 0.3995 | 1841 |
| ch82A1C10 | 2.763 | 2681 |
| ch116G1G4 | 0.6858 | 2236 |
| ch116F1G6 | 0.8631 | 1166 |
| ch110B4H5 | 2.736 | 1818 |
| ch108C4C12 | 0.4626 | 2043 |
| ch101B3G3 | 4.852 | 3935 |

Cell line
Cynomolgus
HEK293T-cynoGPRC5D

| Clone No. | EC₅₀ (nM) | Top |
|---|---|---|
| hIgG1 | NA | NA |
| ATG596 | 26.63 | 22470 |
| ch12G12C2 | 5.942 | 28162 |
| ch20C4E1 | 11.54 | 45790 |
| ch15D6G2 | 19.26 | 122914 |
| ch28H9C12 | 14.75 | 65875 |
| ch26E2H6 | 40.36 | 51309 |
| ch98A3A8 | 9.78 | 46773 |
| ch96C9E7 | 8.254 | 34331 |
| ch90G6H3 | 13.64 | 61620 |
| ch89F11E3 | 278.1 | 40458 |
| ch87A3G8 | 9.738 | 42156 |
| ch110B4H5 | 4.867 | 27638 |
| ch108C4C12 | 3.871 | 39746 |
| ch101B3G3 | 9.196 | 68857 |

Cell line
Mouse
HEK293T-mGPRC5D

| Clone No. | EC₅₀ (nM) | Top |
|---|---|---|
| hIgG1 | NA | NA |
| ATG596 | 25.48 | 45573 |
| ch12G12C2 | 994.9 | 1484328 |
| ch20C4E1 | 3944 | 5062925 |
| ch15D6G2 | 592.1 | 647783 |
| ch28H9C12 | 110.2 | 26546 |
| ch26E2H6 | 184.4 | 45680 |
| ch98A3A8 | 69.61 | 26036 |
| ch96C9E7 | 18.46 | 19197 |
| ch90G6H3 | 12.27 | 55272 |
| ch89F11E3 | 14.18 | 77282 |
| ch87A3G8 | 14.98 | 76228 |
| ch83A9B8 | 47.71 | 29566 |
| ch116G1G4 | 14.81 | 40867 |
| ch110B4H5 | 14.21 | 42903 |
| ch108C4C12 | 108.8 | 25458 |
| ch101B3G3 | 38.6 | 57613 |

### Example 3. Antibody Characterization: T-Cell Dependent Cytotoxicity

3.1 Bispecific hIgG-scFv Antibodies Generation

Bispecific antibodies were generated to evaluate the T cell dependent cytotoxicity potential mediated by all the candidates. hIgG-scFv was used as the format of the generated bispecific antibodies. Each bispecific antibody comprises a chimeric antibody generated in Example 2 and a scFv targeting CD3, wherein the scFv targeting CD3, comprising a VH region comprising an amino acid sequence as set forth in SEQ ID NO: 87 and a VL region comprising an amino acid sequence as set forth in SEQ ID NO: 88, was linked to the C-terminal of the Fc domain of the chimeric antibody by a (GGGGS)₃ liker. LALA mutation (L234A, L235A, according to Eu numbering) was introduced to each bispecific antibody to abolish the Fc receptor binding capability. The resulting bispecific antibodies are referred to herein as Bis-36A3G8, Bis-28H9C12, Bis-26E2H6, Bis-20C4E1, Bis-15D6G2, Bis-12G12C2, Bis-71F8H2, Bis-64G9C1, Bis-98A3A8, Bis-96C9E7, Bis-90G6H3, Bis-89F11E3, Bis-87A3G8, Bis-83A9B8, Bis-82A1C10, Bis-119E4B4, Bis-116G1G4, Bis-116F1G6, Bis-110B41H5, Bis-108C4C12, and Bis-101B3G3, where the prefix “Bis-” indicates “bispecific”, and the following indicates the hybridoma antibody clone from which the chimeric antibody was derived. For example, “Bis-83A9B8” indicates that it is a bispecific antibody comprising a chimeric antibody from the hybridoma antibody clone 83A9B8 and a scFv targeting CD3 (which comprises a VH region comprising an amino acid sequence as set forth in SEQ ID NO: 87 and a VL region comprising an amino acid sequence as set forth in SEQ ID NO: 88).

3.2 T-Cell Dependent Cytotoxicity of Bispecific hIgG-scFv Antibodies on CHOS Cells

The T-cell dependent cytotoxicity of the bispecific hIgG-scFv antibodies on CHOS cells was tested by FACS analysis. The protocol for FACS analysis is described as follows:
(a) CD3⁺ T cells were isolated from normal donors.
(b) 1×10⁴ target cells (CHOS-hGPRC5D-GFP) and 1×10⁵ T cells were added to wells of 96-well U-bottom plates (10:1 Effector:Target ratio).
(c) After target cells and T cells were mixed, final concentration 100 nM with 7 data points by 1:3 serial dilution of bispecific hIgG-scFv antibodies were added to each well.
(d) The plates were incubated at 37° C. with 5% CO₂ for 48 hours.
(e) Two days (48 hours) later, the plates were centrifuged and analyzed by FACS.

As shown in Table 12 and FIG. 2, several of selected bispecific antibodies showed potent T-cell dependent cytotoxicity effect on CHOS-hGPRC5D cells.

TABLE 12
T-cell dependent cytotoxicity effect of selected
antibodies on CHOS-hGPRC5D cells.

| Clone No. | Top killing % | EC₅₀ (nM) |
|---|---|---|
| IgG1 | 12.07 | Unstable |
| Bis-83A9B8 | 66.9 | 1.897 |
| Bis-108C4C12 | 67.93 | 1.439 |
| Bis-116G1G4 | 69.25 | 1.876 |
| Bis-116F1G6 | 71.81 | 1.658 |
| Bis-15D6G2 | 45.86 | 6.693 |
| Bis-98A3A8 | 64.44 | 2.95 |
| Bis-96C9E7 | 66.19 | 2.337 |
| Bis-110B4H5 | 61.52 | 3.615 |
| Bis-26E2H6 | 79.12 | 30.54 |

### 3.3 Bispecific Crossmab Antibodies Generation

To further evaluate the cytotoxicity mediated by the antibody candidates, another serial of bispecific antibody was constructed using Crossmab format as described in WO 2009/080253. In general, each of the generated bispecific Crossmab antibodies is an IgG-like bispecific antibody, comprises an anti-GPRC5D Fab domain, an anti-CD3 Fab domain, and constant domains, wherein the anti-GPRC5D Fab domain comprises a GPRC5D binding moiety comprising the VH and VL regions of each of the hybridoma antibody clones screened in Example 1; the anti-CD3 Fab domain comprises a CD3 binding moiety comprising a VH region comprising an amino acid sequence as set forth in SEQ ID NO: 95 and a VL region comprising an amino acid sequence as set forth in SEQ ID NO: 96, and the CH1 and CL regions of the anti-CD3 Fab domain were replaced by each other; the constant regions are human IgG1 constant region, and LALA mutation (L234A, L235A, Eu numbering) was introduced to the IgG1 Fc region to abolish the Fc receptor binding. In addition, knob-in-hole mutation (S354C, T366W on CH3 of a first heavy chain, Y349C, T366S, L368A, Y407V on CH3 of a second heavy chain) was introduced to avoid heavy chain mismatch. ATG596 was used to construct bispecific antibody with Crossmab format as a positive control. The bispecific Crossmab ATG596 (i.e., Bis-ATG596-CM) only differs from the tested bispecific Crossmab antibodies in the amino acid sequences of the VH and VL regions of the anti-GPRC5D Fab domain. JNJ-64407564 was also used as a positive control. The construct and amino acid sequences of JNJ-64407564 can be obtained from WO2018017786A2. In particular, JNJ-64407564 comprises a GPRC5D binding moiety comprising a VH region as set forth in SEQ JD NO: 199 and a VL region as set forth in SEQ ID NO: 200, and a CD3 binding moiety comprising a VH region as set forth in SEQ ID NO: 95 and a VL region as set forth in SEQ ID NO: 96.

The resulting bispecific antibodies are referred to as Bis-36A3G8-CM, Bis-28H9C12-CM, Bis-26E2H6-CM, Bis-20C4E1-CM, Bis-15D6G2-CM, Bis-12G12C2-CM, Bis-71F8H2-CM, Bis-64G9C1-CM, Bis-98A3A8-CM, Bis-96C9E7-CM, Bis-90G6H3-CM, Bis-89F11E3-CM, Bis-87A3G8-CM, Bis-83A9B8-CM, Bis-82A1C10-CM, Bis-119E4B4-CM, Bis-116G1G4-CM, Bis-116F1G6-CM, Bis-110B4H5-CM, Bis-108C4C12-CM, and Bis-101B3G3-CM, where the prefix “Bis-” indicates “bispecific”, the middle indicates the hybridoma antibody clone, and the suffix “-CM” indicates “Crossmab”. For example, “Bis-108C4C12-CM” indicates that it is a bispecific Crossmab antibody comprising a GPRC5D binding moiety comprising the VH and VL regions of hybridoma antibody clone 108C4C12.

### 3.4 T-Cell Dependent Cytotoxicity of Bispecific Crossmab Antibodies on MM.1S Cells

The cytotoxicity of the bispecific Crossmab antibodies on MM.1S cells was tested by FACS analysis. The protocol for FACS analysis is described as follows:
(a) CD3⁻ T cells were isolated from normal donors.
(b) 1×10⁴ target cells (CFSE-labeled MM.1S) and 1×10⁵ T cells were added to wells of 96-well U-bottom plates (10:1 Effector:Target ratio).
(c) After target and T cells were mixed, final concentration 5 nM with 9 data points by 1:4 serial dilution of bispecific Crossmab antibodies were added to each well.
(d) The plates were incubated at 37° C. with 5% CO₂ for overnight.
(e) After incubation, the plates were centrifuged and analyzed by FACS.

As shown in Table 13 and FIG. 3, all selected antibodies showed potent T-cell dependent cytotoxicity on MM.1S cells. Selected antibodies showed comparable max cytotoxicity on MM.1S cells, and three clones (i.e., Bis-108C4C12-CM, Bis-98A3A8-CM, and Bis-96C9E7-CM) had lower EC₅₀ compared with benchmark antibody, JNJ-64407564 and Bis-ATG596-CM.

TABLE 13
T-cell dependent cytotoxicity effect of
selected antibodies on MM.1S cells.

| Clone No. | Top killing % | EC₅₀ (nM) |
|---|---|---|
| hIgG1 | 3.125 | 21.71 |
| JNJ-64407564 | 83.07 | 0.1002 |
| Bis-ATG596-CM | 82.31 | 0.1743 |
| Bis-108C4C12-CM | 87.25 | 0.01081 |
| Bis-98A3A8-CM | 87.52 | 0.008194 |
| Bis-96C9E7-CM | 86.78 | 0.008085 |

### Example 4. Antibody Characterization: In Vivo Efficacy

### 4.1 Human PBMCs Humanized MM.1S Mouse Model Generation

To assess the activity of bispecific antibodies in vivo, human PBMCs humanized MM.1S model was used. Each NCG (NOD CRISPR Prkdc Il2r gamma) mouse received 1×10⁷ MM.1S human MM (Multiple myeloma) cells implanted SC (Subcutaneous Injection) in the right flank. When the tumor volume reached 60-80 mm³, animals received an I.V. injection of 1×10⁷ human PBMCs. Mice were randomized to groups with similar starting tumor volume when tumor volume reached 100-140 mm³, on the same day, each mouse received I.V. administration of test articles. Treatments were administered in one dose per three days, JNJ-64407564 and Bis-ATG596-CM were used as the benchmark antibodies. Tumor growth inhibition mean was calculated by using the equation below, wherein Mean (Control) indicates the tumor volume mean of the mice treated with hIgG isotype control; Mean (Control D0) indicates the tumor volume mean of the mice treated with hIgG isotype control at Day 0; Mean (Test) indicates the tumor volume mean of the mice treated with the tested bispecific antibodies; Mean (Test D0) indicates the tumor volume mean of the mice treated with the tested bispecific antibodies at Day 0.

$$Mean \% Δ Inhibition = \frac{\begin{matrix}( Mean ( Control ) - Mean ( Control D 0 ) ) - \\ ( Mean ( Test ) - Mean ( Test D 0 ) )\end{matrix}}{Mean ( Control ) - Mean ( Control D 0 )} \times 100 \%$$

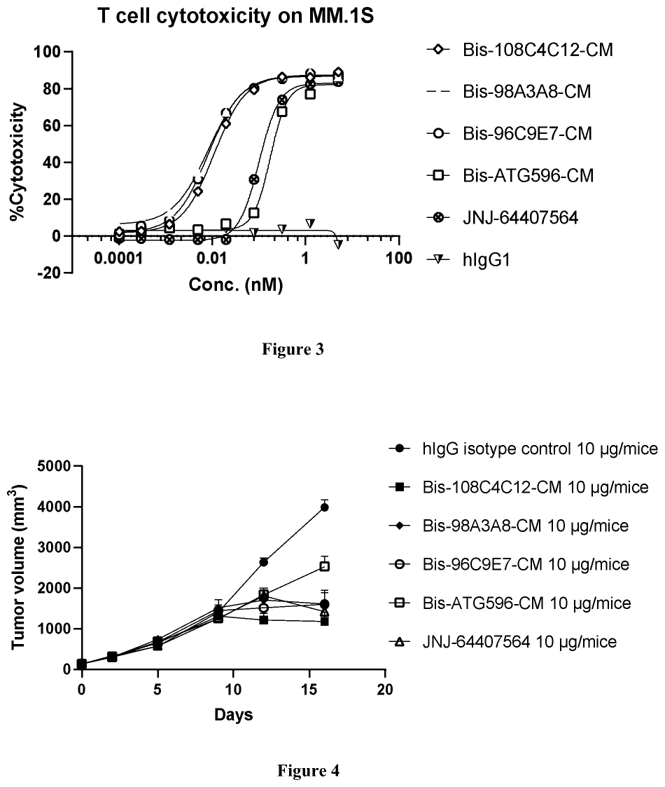

As shown in Table 14 and FIG. 4, most of the candidates exhibited more potent in vivo anti-tumor efficacy than Bis-ATG596-CM in the same format suggesting great in vivo therapeutic potential. Besides, Bis-108C4C12-CM even showed better efficacy than the benchmark antibody JNJ-64407564.

TABLE 14
Tumor growth inhibition mean showing the
in vivo efficacy of selected antibodies.

| | Clone No. | | | | | |
|---|---|---|---|---|---|---|
| hIgG isotype control | D 0 | D 2 | D 5 | D 9 | D 12 | D 16 |
| Bis-108C4C12-CM | | 14.43% | −19.71% | 8.68% | 56.76% | 72.89% |
| Bis-98A3A8-CM | | 16.15% | −40.13% | −7.87% | 37.10% | 61.71% |
| Bis-96C9E7-CM | | 12.67% | −23.76% | −2.22% | 44.78% | 61.95% |
| Bis-ATG596-CM | | 11.86% | −25.31% | 12.99% | 31.97% | 37.94% |
| JNJ-64407564 | | 19.60% | −2.57% | 12.09% | 33.01% | 66.75% |

### Example 5. Antibody Characterization: CAR-T

To evaluate the cytotoxicity efficacy of chimeric antigen receptor (CAR) T cells generated by selected antibodies, CARs were constructed and transfected into T cells, and then incubated with NCI-H929 cells. BMK-203 was used as a benchmark control. The protocol is described as follows. 5.1 Plasmid transfection and recombinant virus collection

Day 0: 293 T cells were collected and counted when cell confluence reached 80%. 8.2×10\hat{ }6 293T cells suspensions were seeded into a T75 flask. The cell plates were returned into a 37° C., 5% CO₂ incubator.

Day 1: After 24 hours incubation, cell confluence reached >90%. The media was removed and replaced with 11 mL fresh Opti-MEM medium.

Transfection System Preparation: Tuba A: 2 ml Opti-MEEM+total 25 μg DNA (12.5 μg CAR vector+7.5 μg pGP+5 μg VSV-G). Tuba B: 2 ml Opti-MEM+75 μg PEI. Tube A was added to Tube B and mixed well, The mixture was incubated at room temperature for 20 mins. The mixture was added into 293T cells slowly, incubated at 37° C. and 5% CO₂ for 6 hours. The media was removed and replaced with 15 mL fresh complete DMEM medium (10% FBS+1% P/S).

Day 3: After 48 hours post-transfection, the entire viral supernatant was harvested and filtered with 0.45 μm filter, and stored at 4° C. The media was then removed and replaced with 15 mL fresh complete DMEM medium, incubated at 37° C. and 5% CO₂ overnight.

Day 4: After 72 hours post-transfection, the entire viral supernatant was harvested and filtered with 0.45 km filter and combined with the previous viral supernatant. The supernatants were centrifuged at 20000×g, 4° C. for 4 hours. The supernatant was discarded and resuspended in 400 μl X-VIVO⁺⁺⁺ medium (5 ng/ml IL-2+10 ng/ml IL-7+5 ng/ml IL-15).

### 5.2 Spinoculation

CD3⁺ T cells were isolated from 50 M frozen human PBMC and stimulated with CD3/CD28 Dynabeads (Gibco, #11131 D) at a 1:1 ratio and grown in X-VIVO⁺⁺⁺ medium.

Day 1: after three days of activation, T cells were spinoculated with CAR lentivirus. The prepared CAR lentivirus expressed a CAR comprising an extracellular antigen-binding region having the VH and VL region amino acid sequences of each selected antibody, a CD8 transmembrane region having the amino acid sequence of SEQ ID NO: 203, a 4-1BB intracellular signal region having the amino acid sequence of SEQ ID NO: 204, and a CD3ζ intracellular signal region having the amino acid sequence as set forth in SEQ ID NO: 205.

Concentrated virus and Polybrene Reagent (final concentration 8 μg/ml) were added to 5×10\hat{ }5 activated T cells (cultured in 2 ml X-VIVO⁺⁺⁺ medium) in a 12-well plate (MOI≈100:1), total 700-800 μl volume per well.

The mixtures were centrifuged at 1000×g, room temperature for 2 hours, and then returned to a 37° C., 5% CO₂ incubator. After 24 hours of incubation, replaced with fresh X-VIVO⁺⁺⁺ medium.

Day 5: after 4 days of infection, CD3/CD28 Dynabeads were removed and transduction efficiency (GFP positive %) was detected.

CAR-T cytotoxicity assay was conducted on target cells.

### 5.3 Cytotoxicity Assay

Cell Trace-Far red labeled 3×10⁴NCI-H929 cells were added to a 96-well U-bottom plate (100 μl/well).

100 μl/well CAR-T or vehicle CD3 T cells were added to the wells at a specific effector-to-tumor cell (E:T) ratios, E=GFP positive cells.

After 48 hours of coculture, live cell number of Far Red signaling positive tumor cells were detected by FACS.

The Cytotoxicity was calculated as follows:

% Cytotoxicity/CD3 T=(Far Red⁺ cell number of CD3 T group−Far Red⁺ cell number of CAR-T group)/Far Red⁺ cell number of CD3 T group*100%

### 5.4 Results

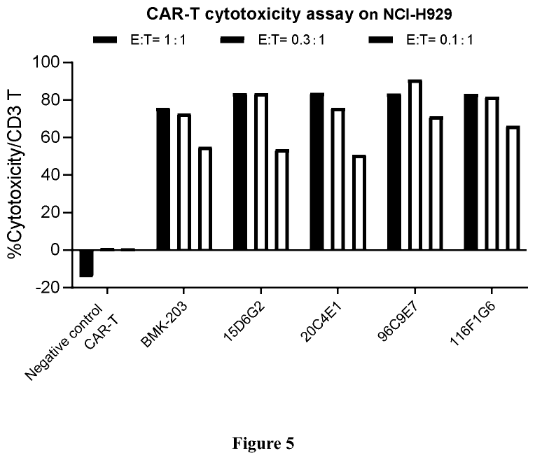

As shown in FIG. 5, all of our test antibodies showed potent CAR-T cytotoxicity on NCI-H929 cells, with comparable or better cell killing efficacy compared with the benchmark control BMK-203.

What claimed is:

1. An antibody or antigen-binding fragment thereof which binds to GPRC5D, comprising:
one or two or three heavy chain complementarity determining regions (HCDR1, HCDR2 and/or HCDR3) contained within any one of the heavy chain variable (VH) region sequences selected from the group consisting of SEQ ID NOs: 7, 15, 23, 31, 39, 47, 55, 63, 71, 79, 103, 111, 119, 127, 135, 143, 151, 159, 175, 183, and 191; and/or
one or two or three light chain complementarity determining regions (LCDR1, LCDR2 and LCDR3) contained within any one of the light chain variable (VL) region sequences selected from the group consisting of SEQ ID NOs: 8, 16, 24, 32, 40, 48, 56, 64, 72, 80, 104, 112, 120, 128, 136, 144, 152, 160, 176, 184, and 192.

2. The antibody or antigen-binding fragment thereof of claim 1, wherein the antibody or antigen-binding fragment thereof comprises at least one heavy or light chain complementarity determining region (CDR) comprising an amino acid sequence selected from the group consisting of SEQ ID NOs: 1, 2, 3, 4, 5, 6, 9, 10, 11, 12, 13, 14, 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 97, 98, 99, 100, 101, 102, 105, 106, 107, 108, 109, 110, 113, 114, 115, 116, 117, 118, 121, 122, 123, 124, 125, 126, 129, 130, 131, 132, 133, 134, 137, 138, 139, 140, 141, 142, 145, 146, 147, 148, 149, 150, 153, 154, 155, 156, 157, 158, 169, 170, 171, 172, 173, 174, 177, 178, 179, 180, 181, 182, 185, 186, 187, 188, 189, and 190.

3. The antibody or antigen-binding fragment thereof of claim 1 or 2, comprising a VH region comprising one or two or three of HCDR1, HCDR2 and HCDR3 comprising an amino acid sequence selected from the group consisting of SEQ ID NOs: 1, 2, 3, 9, 10, 11, 17, 18, 19, 25, 26, 27, 33, 34, 35, 41, 42, 43, 49, 50, 51, 57, 58, 59, 65, 66, 67, 73, 74, 75, 97, 98, 99, 105, 106, 107, 113, 114, 115, 121, 122, 123, 129, 130, 131, 137, 138, 139, 145, 146, 147, 153, 154, 155, 169, 170, 171, 177, 178, 179, 185, 186, and 187.

4. The antibody or antigen-binding fragment thereof of any one of the preceding claims, comprising a VL region comprising one or two or three of LCDR1, LCDR2 and LCDR3 comprising an amino acid sequence selected from the group consisting of SEQ ID NOs: 4, 5, 6, 12, 13, 14, 20, 21, 22, 28, 29, 30, 36, 37, 38, 44, 45, 46, 52, 53, 54, 60, 61, 62, 68, 69, 70, 76, 77, 78, 100, 101, 102, 108, 109, 110, 116, 117, 118, 124, 125, 126, 132, 133, 134, 140, 141, 142, 148, 149, 150, 156, 157, 158, 172, 173, 174, 180, 181, 182, 188, 189, and 190.

5. The antibody or antigen-binding fragment thereof of any one of the preceding claims, comprising:
i. a HCDR1 comprising an amino acid sequence selected from the group consisting of SEQ ID NOs: 1, 9, 17, 25, 33, 41, 49, 57, 65, 73, 97, 105, 113, 121, 129, 137, 145, 153, 169, 177, and 185;
ii. a HCDR2 comprising an amino acid sequence selected from the group consisting of SEQ ID NOs: 2, 10, 18, 26, 34, 42, 50, 58, 66, 74, 98, 106, 114, 122, 130, 138, 146, 154, 170, 178, and 186; and
iii. a HCDR3 comprising an amino acid sequence selected from the group consisting of SEQ ID NOs: 3, 11, 19, 27, 35, 43, 51, 59, 67, 75, 99, 107, 115, 123, 131, 139, 147, 155, 171, 179, and 187.

6. The antibody or antigen-binding fragment thereof of any one of the preceding claims, comprising:
i. a LCDR1 comprising an amino acid sequence selected from the group consisting of SEQ ID NOs: 4, 12, 20, 28, 36, 44, 52, 60, 68, 76, 100, 108, 116, 124, 132, 140, 148, 156, 172, 180, and 188;
ii. a LCDR2 comprising an amino acid sequence selected from the group consisting of SEQ ID NOs: 5, 13, 21, 29, 37, 45, 53, 61, 69, 77, 101, 109, 117, 125, 133, 141, 149, 157, 173, 181, and 189; and
iii. a LCDR3 comprising an amino acid sequence selected from the group consisting of SEQ ID NOs: 6, 14, 22, 30, 38, 46, 54, 62, 70, 78, 102, 110, 118, 126, 134, 142, 150, 158, 174, 182, and 190.

7. The antibody or antigen-binding fragment thereof of any one of the preceding claims, comprising:
i. a HCDR1 comprising an amino acid sequence as set forth in SEQ ID NO: 1, a HCDR2 comprising an amino acid sequence as set forth in SEQ ID NO: 2, and a HCDR3 comprising an amino acid sequence as set forth in SEQ ID NO: 3;
ii. a HCDR1 comprising an amino acid sequence as set forth in SEQ ID NO: 9, a HCDR2 comprising an amino acid sequence as set forth in SEQ ID NO: 10, and a HCDR3 comprising an amino acid sequence as set forth in SEQ ID NO: 11;
iii. a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
iv. a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
v. a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
vi. a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
vii. a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
viii. a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
ix. a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
x. a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
xi. a HCDR1 comprising an amino acid sequence as set forth in SEQ ID NO: 97, a HCDR2 comprising an amino acid sequence as set forth in SEQ ID NO: 98, and a HCDR3 comprising an amino acid sequence as set forth in SEQ ID NO: 99;
xii. a HCDR1 comprising an amino acid sequence as set forth in SEQ ID NO: 105, a HCDR2 comprising an amino acid sequence as set forth in SEQ ID NO: 106, and a HCDR3 comprising an amino acid sequence as set forth in SEQ ID NO: 107;
xiii. a HCDR1 comprising an amino acid sequence as set forth in SEQ ID NO: 113, a HCDR2 comprising an amino acid sequence as set forth in SEQ ID NO: 114, and a HCDR3 comprising an amino acid sequence as set forth in SEQ ID NO: 115;
xiv. a HCDR1 comprising an amino acid sequence as set forth in SEQ ID NO: 121, a HCDR2 comprising an amino acid sequence as set forth in SEQ ID NO: 122, and a HCDR3 comprising an amino acid sequence as set forth in SEQ ID NO: 123;
xv. a HCDR1 comprising an amino acid sequence as set forth in SEQ ID NO: 129, a HCDR2 comprising an amino acid sequence as set forth in SEQ ID NO: 130, and a HCDR3 comprising an amino acid sequence as set forth in SEQ ID NO: 131;
xvi. a HCDR1 comprising an amino acid sequence as set forth in SEQ ID NO: 137, a HCDR2 comprising an amino acid sequence as set forth in SEQ ID NO: 138, and a HCDR3 comprising an amino acid sequence as set forth in SEQ ID NO: 139;
xvii. a HCDR1 comprising an amino acid sequence as set forth in SEQ ID NO: 145, a HCDR2 comprising an amino acid sequence as set forth in SEQ ID NO: 146, and a HCDR3 comprising an amino acid sequence as set forth in SEQ ID NO: 147;
xviii. a HCDR1 comprising an amino acid sequence as set forth in SEQ ID NO: 153, a HCDR2 comprising an amino acid sequence as set forth in SEQ ID NO: 154, and a HCDR3 comprising an amino acid sequence as set forth in SEQ ID NO: 155;
xix. a HCDR1 comprising an amino acid sequence as set forth in SEQ ID NO: 169, a HCDR2 comprising an amino acid sequence as set forth in SEQ ID NO: 170, and a HCDR3 comprising an amino acid sequence as set forth in SEQ ID NO: 171;
xx. a HCDR1 comprising an amino acid sequence as set forth in SEQ ID NO: 177, a HCDR2 comprising an amino acid sequence as set forth in SEQ ID NO: 178, and a HCDR3 comprising an amino acid sequence as set forth in SEQ ID NO: 179; or
xxi. a HCDR1 comprising an amino acid sequence as set forth in SEQ ID NO: 185, a HCDR2 comprising an amino acid sequence as set forth in SEQ ID NO: 186, and a HCDR3 comprising an amino acid sequence as set forth in SEQ ID NO: 187.

8. The antibody or antigen-binding fragment thereof of any one of the preceding claims, comprising:
i. a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
ii. a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
iii. a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
iv. a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
v. a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
vi. a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vii. a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
viii. a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
ix. a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
x. a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
xi. a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142;
xvii. a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150;
xviii. a LCDR1 comprising an amino acid sequence as set forth in SEQ ID NO: 156, a LCDR2 comprising an amino acid sequence as set forth in SEQ ID NO: 157, and a LCDR3 comprising an amino acid sequence as set forth in SEQ ID NO: 158;
xix. a LCDR1 comprising an amino acid sequence as set forth in SEQ ID NO: 172, a LCDR2 comprising an amino acid sequence as set forth in SEQ ID NO: 173, and a LCDR3 comprising an amino acid sequence as set forth in SEQ ID NO: 174;
xx. a LCDR1 comprising an amino acid sequence as set forth in SEQ ID NO: 180, a LCDR2 comprising an amino acid sequence as set forth in SEQ ID NO: 181, and a LCDR3 comprising an amino acid sequence as set forth in SEQ ID NO: 182; or
xxi. a LCDR1 comprising an amino acid sequence as set forth in SEQ ID NO: 188, a LCDR2 comprising an amino acid sequence as set forth in SEQ ID NO: 189, and a LCDR3 comprising an amino acid sequence as set forth in SEQ ID NO: 190.

9. The antibody or antigen-binding fragment thereof of any one of the preceding claims, comprising:
i. a HCDR1 comprising an amino acid sequence as set forth in SEQ ID NO: 1, a HCDR2 comprising an amino acid sequence as set forth in SEQ ID NO: 2, a HCDR3 comprising an amino acid sequence as set forth in SEQ ID NO: 3, a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
ii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
iii. a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
iv. a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
v. a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
vi. a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vii. a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
viii. a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
ix. a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
x. a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
xi. a HCDR1 comprising an amino acid sequence as set forth in SEQ ID NO: 97, a HCDR2 comprising an amino acid sequence as set forth in SEQ ID NO: 98, a HCDR3 comprising an amino acid sequence as set forth in SEQ ID NO: 99, a LCDR1 comprising an amino acid sequence as set forth in SEQ ID NO: 100, a LCDR2 comprising an amino acid sequence as set forth in SEQ ID NO: 101, and a LCDR3 comprising an amino acid sequence as set forth in SEQ ID NO: 102;
xii. a HCDR1 comprising an amino acid sequence as set forth in SEQ ID NO: 105, a HCDR2 comprising an amino acid sequence as set forth in SEQ ID NO: 106, a HCDR3 comprising an amino acid sequence as set forth in SEQ ID NO: 107, a LCDR1 comprising an amino acid sequence as set forth in SEQ ID NO: 108, a LCDR2 comprising an amino acid sequence as set forth in SEQ ID NO: 109, and a LCDR3 comprising an amino acid sequence as set forth in SEQ ID NO: 110;
xiii. a HCDR1 comprising an amino acid sequence as set forth in SEQ ID NO: 113, a HCDR2 comprising an amino acid sequence as set forth in SEQ ID NO: 114, a HCDR3 comprising an amino acid sequence as set forth in SEQ ID NO: 115, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xiv. a HCDR1 comprising an amino acid sequence as set forth in SEQ ID NO: 121, a HCDR2 comprising an amino acid sequence as set forth in SEQ ID NO: 122, a HCDR3 comprising an amino acid sequence as set forth in SEQ ID NO: 123, a LCDR1 comprising an amino acid sequence as set forth in SEQ ID NO: 124, a LCDR2 comprising an amino acid sequence as set forth in SEQ ID NO: 125, and a LCDR3 comprising an amino acid sequence as set forth in SEQ ID NO: 126;
xv. a HCDR1 comprising an amino acid sequence as set forth in SEQ ID NO: 129, a HCDR2 comprising an amino acid sequence as set forth in SEQ ID NO: 130, a HCDR3 comprising an amino acid sequence as set forth in SEQ ID NO: 131, a LCDR1 comprising an amino acid sequence as set forth in SEQ ID NO: 132, a LCDR2 comprising an amino acid sequence as set forth in SEQ ID NO: 133, and a LCDR3 comprising an amino acid sequence as set forth in SEQ ID NO: 134;
xvi. a HCDR1 comprising an amino acid sequence as set forth in SEQ ID NO: 137, a HCDR2 comprising an amino acid sequence as set forth in SEQ ID NO: 138, a HCDR3 comprising an amino acid sequence as set forth in SEQ ID NO: 139, a LCDR1 comprising an amino acid sequence as set forth in SEQ ID NO: 140, a LCDR2 comprising an amino acid sequence as set forth in SEQ ID NO: 141, and a LCDR3 comprising an amino acid sequence as set forth in SEQ ID NO: 142;
xvii. a HCDR1 comprising an amino acid sequence as set forth in SEQ ID NO: 145, a HCDR2 comprising an amino acid sequence as set forth in SEQ ID NO: 146, a HCDR3 comprising an amino acid sequence as set forth in SEQ ID NO: 147, a LCDR1 comprising an amino acid sequence as set forth in SEQ ID NO: 148, a LCDR2 comprising an amino acid sequence as set forth in SEQ ID NO: 149, and a LCDR3 comprising an amino acid sequence as set forth in SEQ ID NO: 150;
xviii. a HCDR1 comprising an amino acid sequence as set forth in SEQ ID NO: 153, a HCDR2 comprising an amino acid sequence as set forth in SEQ ID NO: 154, a HCDR3 comprising an amino acid sequence as set forth in SEQ ID NO: 155, a LCDR1 comprising an amino acid sequence as set forth in SEQ ID NO: 156, a LCDR2 comprising an amino acid sequence as set forth in SEQ ID NO: 157, and a LCDR3 comprising an amino acid sequence as set forth in SEQ ID NO: 158;
xix. a HCDR1 comprising an amino acid sequence as set forth in SEQ ID NO: 169, a HCDR2 comprising an amino acid sequence as set forth in SEQ ID NO: 170, a HCDR3 comprising an amino acid sequence as set forth in SEQ ID NO: 171, a LCDR1 comprising an amino acid sequence as set forth in SEQ ID NO: 172, a LCDR2 comprising an amino acid sequence as set forth in SEQ ID NO: 173, and a LCDR3 comprising an amino acid sequence as set forth in SEQ ID NO: 174;
xx. a HCDR1 comprising an amino acid sequence as set forth in SEQ ID NO: 177, a HCDR2 comprising an amino acid sequence as set forth in SEQ ID NO: 178, a HCDR3 comprising an amino acid sequence as set forth in SEQ ID NO: 179, a LCDR1 comprising an amino acid sequence as set forth in SEQ ID NO: 180, a LCDR2 comprising an amino acid sequence as set forth in SEQ ID NO: 181, and a LCDR3 comprising an amino acid sequence as set forth in SEQ ID NO: 182; or
xxi. a HCDR1 comprising an amino acid sequence as set forth in SEQ ID NO: 185, a HCDR2 comprising an amino acid sequence as set forth in SEQ ID NO: 186, a HCDR3 comprising an amino acid sequence as set forth in SEQ ID NO: 187, a LCDR1 comprising an amino acid sequence as set forth in SEQ ID NO: 188, a LCDR2 comprising an amino acid sequence as set forth in SEQ ID NO: 189, and a LCDR3 comprising an amino acid sequence as set forth in SEQ ID NO: 190.

10. The antibody or antigen-binding fragment thereof of any one of the preceding claims, comprising a VH region having an amino acid sequence as set forth in SEQ ID NOs: 7, 15, 23, 31, 39, 47, 55, 63, 71, 79, 103, 111, 119, 127, 135, 143, 151, 159, 175, 183, or 191, or a homologous sequence thereof having at least 80% sequence identity to SEQ ID NOs: 7, 15, 23, 31, 39, 47, 55, 63, 71, 79, 103, 111, 119, 127, 135, 143, 151, 159, 175, 183, or 191.

11. The antibody or antigen-binding fragment thereof of any one of the preceding claims, comprising a VL region having an amino acid sequence as set forth in SEQ ID NOs: 8, 16, 24, 32, 40, 48, 56, 64, 72, 80, 104, 112, 120, 128, 136, 144, 152, 160, 176, 184, or 192, or a homologous sequence thereof having at least 80% sequence identity to SEQ ID NOs: 8, 16, 24, 32, 40, 48, 56, 64, 72, 80, 104, 112, 120, 128, 136, 144, 152, 160, 176, 184, or 192.

12. The antibody or antigen-binding fragment thereof of any one of the preceding claims, comprising a VH/VL amino acid sequence pair selected from the group consisting of SEQ ID NOs: 7/8, 15/16, 23/24, 31/32, 39/40, 47/48, 55/56, 63/64, 71/72, 79/80, 103/104, 111/112, 119/120, 127/128, 135/136, 143/144, 151/152, 159/160, 175/176, 183/184, or 191/192.

13. The antibody or antigen-binding fragment thereof of any one of the preceding claims, further comprising one or more amino acid residue substitutions or modifications yet retains binding affinity to GPRC5D.

14. The antibody or antigen-binding fragment thereof of claim 13, wherein at least one of the substitutions or modifications is in one or more of the CDR sequences of the VH region or VL region.

15. The antibody or antigen-binding fragment thereof of claim 13, wherein at least one of the substitutions or modifications is in one or more of the non-CDR sequences of the VH region or VL region.

16. The antibody or antigen-binding fragment of the proceeding claims, further comprising one or more non-natural amino acid (NNAA) substitution.

17. The antibody or antigen-binding fragment of claim 16, wherein the NNAA is capable of being conjugated.

18. The antibody or antigen-binding fragment thereof of any one of the preceding claims, having one or more properties selected from the group consisting of:
i. being with cross reactivity against human, cynomolgus and mouse GPRC5D;
ii. being capable of binding to human GPRC5D, cynomolgus GPRC5D, or mouse GPRC5D as measured by FACS assay;
iii. binding to human GPRC5D at an EC₅₀ of no more than 10 nM as measure as measured by FACS assay;
iv. showing T-cell dependent cytotoxicity effect as measured by FACS assay.

19. An antibody or antigen-binding fragment thereof, which competes for binding to GPRC5D with the antibody or antigen-binding fragment thereof any of the preceding claims.

20. The antibody or antigen-binding fragment thereof of any one of the preceding claims, which is a chimeric, a humanized or a human antibody or antigen-binding fragment thereof.

21. The antibody or antigen-binding fragment thereof of any one of the preceding claims, which is a labeled antibody, a bivalent antibody, an anti-idiotypic antibody or a fusion protein.

22. The antibody or antigen-binding fragment thereof of any one of the preceding claims, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

23. The antibody or antigen-binding fragment thereof of any one of the preceding claims, further comprising an Fc region, optionally an Fc region of human immunoglobulin (Ig), or optionally an Fc region of human IgG.

24. The antibody or antigen-binding fragment thereof of claim 23, wherein the Fc region is derived from human IgG1, IgG2, IgG3, or IgG4.

25. The antibody or antigen-binding fragment of claim 24, wherein the Fc region comprises an amino acid sequence selected from the group consisting of SEQ ID NOs: 161-163.

26. The antibody or antigen-binding fragment thereof of any one of the preceding claims, wherein the light chain is a λ light chain or a κ light chain.

27. The antibody or antigen-binding fragment thereof of any one of the preceding claims, which is a bispecific or multi-specific antibody or antigen-binding fragment thereof.

28. The antibody or antigen-binding fragment thereof of claim 27, which is capable of binding to one or more additional antigens other than GPRC5D, or a second epitope on GPRC5D.

29. The antibody or antigen-binding fragment thereof of claim 28, wherein the one or more additional antigens other than GPRC5D are selected from the group consisting of KRAS, ERK, XPO, mTORC1/2, PAK4, NAMPT, ATR, EGFR, FGFR, VEGF, LILRB, c-MET, Her2, Her3, CTLA4, GITA, CD112R, CD2, CD3, CD7, CD16, CD19, CD20, CD24, CD27, CD30, CD34, CD37, CD38, CD39, CD70, CD73, CD83, CD28, CD80 (B7-1), CD86 (B7-2), CD40, CD40L (CD154), CD47, SIRPα, CD122, CD137, CD137L, OX40 (CD134), OX40L (CD252), BCMA (e.g., BCMA02), FcRH5, PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), 137-H4 (VTCN1), B7-H5, BTLA (CD272), BTLA, CD160, CTLA-4 (CD152), IDO1, IDO2, ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), and TGFβ.

30. The antibody or antigen-binding fragment thereof of any one of the preceding claims, which is a bispecific antibody or antigen-binding fragment thereof that binds to GPRC5D and CD3.

31. The antibody or antigen-binding fragment thereof of claim 30, wherein the bispecific antibody or antigen-binding fragment thereof comprises a GPRC5D binding moiety and a CD3 binding moiety.

32. The antibody or antigen-binding fragment thereof of claim 31, wherein the CD3 binding moiety comprises
one or two or three heavy chain complementarity determining regions (HCDR1, HCDR2 and/or HCDR3) contained within the heavy chain variable (VH) region sequence of SEQ ID NO: 87 or 95; and/or
one or two or three light chain complementarity determining regions (LCDR1, LCDR2 and/or LCDR3) contained with the light chain variable (VL) region sequence of SEQ ID NO: 88 or 96.

33. The antibody or antigen-binding fragment thereof of claim 32, wherein the CD3 binding moiety comprises:
a HCDR1 comprising an amino acid sequence as set forth in SEQ ID NO: 81 or 89, a HCDR2 comprising an amino acid sequence as set forth in SEQ ID NO: 82 or 90, and a HCDR3 comprising an amino acid sequence as set forth in SEQ ID NO: 83 or 91;
and/or a LCDR1 comprising an amino acid sequence as set forth in SEQ ID NO: 84 or 92, a LCDR2 comprising an amino acid sequence as set forth in SEQ ID NO: 85 or 93, and a LCDR3 comprising an amino acid sequence as set forth in SEQ ID NO: 86 or 94.

34. The antibody or antigen-binding fragment thereof of claim 33, wherein the CD3 binding moiety comprises:
i. a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86; or
ii. a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94.

35. The antibody or antigen-binding fragment thereof of claim 34, wherein the CD3 binding moiety comprises a VH/VL amino acid sequence pair of SEQ ID NOs: 87/88 or 95/96.

36. The antibody or antigen-binding fragment thereof of claim 35, wherein the CD3 binding moiety further comprises a constant domain CL and a constant domain CH1.

37. The antibody or antigen-binding fragment thereof of claim 36, wherein the constant domains CL and CH1 are replaced by each other.

38. The antibody or antigen-binding fragment thereof of any one of claims 27-37, further comprising an Fc region, optionally an Fc region of human immunoglobulin (Ig), or optionally an Fc region of human IgG.

39. The antibody or antigen-binding fragment thereof of claim 38, wherein the Fe region is derived from human IgG1, IgG2, IgG3, or IgG4.

40. The antibody or antigen-binding fragment thereof of claim 38 or 39, wherein the Fc region comprises one or more amino acid substitutions selected from the group consisting of: L234A, L235A, S354C, T366W, Y349C, T366S, L368A, and Y407V (according to EU numbering).

41. The antibody or antigen-binding fragment thereof of claim 40, wherein the Fc region comprises L234A and L235A (according to EU numbering) amino acid substitutions.

42. The antibody or antigen-binding fragment thereof of claim 40, wherein the Fc region of one heavy chain comprises S354C and T366W (according to EU numbering) amino acid substitutions, and the Fc region of the other heavy chain comprises Y349C, T366S, L368A, and Y407V (according to EU numbering) amino acid substitutions.

43. The antibody or antigen-binding fragment thereof of any one of the preceding claims, which is linked to one or more conjugate moieties.

44. The antibody or antigen-binding fragment thereof of claim 43, wherein the conjugate moiety comprises a clearance-modifying agent, a chemotherapeutic agent, a toxin, a radioactive isotope, a lanthanide, a detectable label, a DNA-alkylator, a topoisomerase inhibitor, a tubulin-binder, a purification moiety or other anticancer drugs.

45. The antibody or antigen-binding fragment thereof of claim 43 or 44, wherein the conjugate moiety is covalently attached either directly or via a linker.

46. A chimeric antigen receptor, comprising the antibody or antigen-binding fragment thereof of any one of claims 1-45, a transmembrane region and an intracellular signal region.

47. The chimeric antigen receptor of claim 46, wherein the transmembrane region comprises a transmembrane region of CD3, CD4, CD8 or CD28.

48. The chimeric antigen receptor of claim 46, wherein the intracellular signal region is selected from the group consisting of: an intracellular signal regions sequence of CD3 (e.g. CD3ζ), FcγRI, CD27, CD28, CD137, CD134, MyD88, CD40, CD278, TLRs, or a combination thereof.

49. The chimeric antigen receptor of any one of claims 46-48, wherein the antigen-binding fragment is a scFv.

50. The chimeric antigen receptor of any one of claims 46-49, wherein the chimeric antigen receptor is grafted onto an allogeneic cell, an autologous cell or a xenogeneic cell.

51. The chimeric antigen receptor of any one of claims 46-50, wherein the chimeric antigen receptor is grafted onto an immune effector cell.

52. The chimeric antigen receptor of any one of claims 46-51, wherein the chimeric antigen receptor is grafted onto a T cell, a natural killer cell, a macrophage cell, or a tumor-infiltrating lymphocyte.

53. A pharmaceutical composition comprising the antibody or antigen-binding fragment thereof of any one of claims 1-45, and/or the chimeric antigen receptor of any one of claims 46-52, and one or more pharmaceutically acceptable carriers.

54. An isolated polynucleotide encoding the antibody or antigen-binding fragment thereof of any one of claims 1-45, and/or the chimeric antigen receptor of any one of claims 46-52.

55. A vector comprising the isolated polynucleotide of claim 54.

56. A host expression system comprising the vector of claim 55 or having the polynucleotide of claim 54 integrated into genome thereof.

57. The host expression system of claim 56, which is a microorganism, a yeast, or a mammalian cell, wherein the microorganism is selected from the group consisting of E. coli and B. subtilis, wherein the yeast is Saccharomyces, and wherein the mammalian cell is selected from the group consisting of COS, CHO-S, CHO-K1, HEK-293, and 3T3 cells.

58. A virus comprising the vector of claim 55.

59. A kit comprising the antibody or antigen-binding fragment thereof of any one of claims 1-45 and/or the chimeric antigen receptor of any one of claims 46-52 and/or the pharmaceutical composition of claim 53, and a second therapeutic agent.

60. A method of expressing the antibody or antigen-binding fragment thereof of any one of claims 1-45 or the chimeric antigen receptor of any one of claims 46-52, comprising culturing the host expression system of claim 56 under the condition at which the antibody or antigen-binding fragment of any one of claims 1-45 is expressed.

61. A method of treating, preventing or alleviating a disease, disorder or condition in a subject, comprising administering to the subject a therapeutically effective amount of the antibody or antigen-binding fragment thereof of any one of claims 1-45 and/or the chimeric antigen receptor of any one of claims 46-52 and/or the pharmaceutical composition of claim 53.

62. The method of claim 61, wherein the disease, disorder or condition is cancer.

63. The method of claim 62, wherein the cancer is a solid tumor or hematologic tumor.

64. The method of claim 63, wherein the disease, disorder or condition is a GPRC5D-expressing B cell cancer.

65. The method of any one of claims 61-64, wherein the disease, disorder or condition is selected from the group consisting of lung cancer (e.g., non-small-cell lung cancer (NSCLC), small cell lung cancer (SCLC), adenocarcinoma of the lung, or squamous cell carcinoma of the lung), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), cutaneous or intraocular melanoma, thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple mycloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma.

66. The method of claim 65, wherein the disease, disorder or condition is multiple myeloma.

67. The method of any one of claims 61-66, wherein the subject is human.

68. The method of any one of claims 61-67, wherein the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

69. The method of any one of claims 61-68, wherein the method further includes administering to the subject in need thereof an additional therapeutic agent.

70. The method of claim 69, wherein the additional therapeutic agent is selected from the group consisting of: an active agent, an imaging agent, a cytotoxic agent, and angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, and a microRNA.

71. The method of claim 69, wherein the additional therapeutic agent is administered to the subject in need before, after or simultaneously with the antibody or antigen-binding fragment thereof of any one of claims 1-45 and/or the chimeric antigen receptor of any one of claims 46-52 and/or the pharmaceutical composition of claim 53.

72. A method of inactivating a GPRC5D-expressing cell in vivo or in vitro, comprising contacting the GPRC5D-expressing cell with the antibody or antigen-binding fragment thereof of any one of claims 1-45 and/or the chimeric antigen receptor of any one of claims 46-52 and/or the pharmaceutical composition of claim 53.

73. A method of modulating GPRC5D activity in a GPRC5D-expressing cell, comprising exposing the GPRC5D-expressing cell to the antibody or antigen-binding fragment thereof of any of claims 1-45 and/or the chimeric antigen receptor of any one of claims 46-52 and/or the pharmaceutical composition of claim 53.

74. A method of detecting presence or amount of GPRC5D in a sample, comprising contacting the sample with the antibody or antigen-binding fragment thereof of any of claims 1-45 and/or the chimeric antigen receptor of any one of claims 46-52 and/or the pharmaceutical composition of claim 53, and determining the presence or the amount of GPRC5D in the sample.

75. A method of diagnosing a GPRC5D-related disease, disorder or condition in a subject, comprising: a) obtaining a sample from the subject; b) contacting the sample obtained from the subject with the antibody or antigen-binding fragment thereof of any of claims 1-45 and/or the chimeric antigen receptor of any one of claims 46-52 and/or the pharmaceutical composition of claim 53; c) determining presence or amount of GPRC5D in the sample; and d) correlating the presence or the amount of GPRC5D to existence or status of the GPRC5D-related disease, disorder or condition in the subject.

76. Use of the antibody or antigen-binding fragment thereof of any of claims 1-45 and/or the chimeric antigen receptor of any one of claims 46-52 and/or the pharmaceutical composition of claim 53 in the manufacture of a medicament for treating a GPRC5D-related disease, disorder or condition in a subject.

77. Use of the antibody or antigen-binding fragment thereof of any of claims 1-45 and/or the chimeric antigen receptor of any one of claims 46-52 and/or the pharmaceutical composition of claim 53 in the manufacture of a diagnostic reagent for diagnosing a GPRC5D-related disease, disorder or condition.

78. A kit comprising the antibody or antigen-binding fragment thereof of any of claims 1-45 and/or the chimeric antigen receptor of any one of claims 46-52 and/or the pharmaceutical composition of claim 53, useful in detecting GPRC5D, optionally recombinant GPRC5D, GPRC5D expressed on cell surface, or GPRC5D-expressing cells.
